SEQUENCE LISTING (27 sequences)

SEQ ID NO: 1 - DNA, 1680 bp, Gallus gallus
  source 1..1680: mol_type = unassigned DNA
ctcagatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctaca

SEQ ID NO: 2 - DNA, 473 bp, Homo sapiens
  source 1..473: mol_type = unassigned DNA
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcga

SEQ ID NO: 3 - DNA, 143 bp, Adeno-associated virus
  source 1..143: mol_type = unassigned DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttc

SEQ ID NO: 4 - DNA, 143 bp, Adeno-associated virus
  source 1..143: mol_type = unassigned DNA
aaccggtgagggagagacgcgcgagcgagcgagtgactccggcccgctggtttccagcgggctgcgggcccgaaacgggcccgccggagtcactcgctcgctcgcgcgtctctccctcaccggttgaggtagtgatccccaag

SEQ ID NO: 5 - DNA, 1779 bp, Homo sapiens
  source 1..1779: mol_type = unassigned DNA
atgtggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctg

SEQ ID NO: 6 - DNA, 1767 bp, Mus sp.
  source 1..1767: mol_type = unassigned DNA
atgtgggtcctgatgagctggctggccttcgcggcagggctggtagccggaacacagtgtccagatgggcagttctgccctgttgcctgctgccttgaccagggaggagccaactacagctgctgtaaccctcttctggacacatggcctagaataacgagccatcatctagatggctcctgccagacccatggccactgtcctgctggctattcttgtcttctcactgtgtctgggacttccagctgctgcccgttctctaagggtgtgtcttgtggtgatggctaccactgctgcccccagggcttccactgtagtgcagatgggaaatcctgcttccagatgtcagataaccccttgggtgctgtccagtgtcctgggagccagtttgaatgtcctgactctgccacctgctgcattatggttgatggttcgtggggatgttgtcccatgccccaggcctcttgctgtgaagacagagtgcattgctgtccccatggggcctcctgtgacctggttcacacacgatgcgtttcacccacgggcacccacaccctactaaagaagttccctgcacaaaagaccaacagggcagtgtctttgcctttttctgtcgtgtgccctgatgctaagacccagtgtcccgatgattctacctgctgtgagctacccactgggaagtatggctgctgtccaatgcccaatgccatctgctgttccgaccacctgcactgctgcccccaggacactgtatgtgacctgatccagagtaagtgcctatccaagaactacaccacggatctcctgaccaagctgcctggatacccagtgaaggaggtgaagtgcgacatggaggtgagctgccctgaaggatatacctgctgccgcctcaacactggggcctggggctgctgtccatttgccaaggccgtgtgttgtgaggatcacattcattgctgcccggcagggtttcagtgtcacacagagaaaggaacctgcgaaatgggtatcctccaagtaccctggatgaagaaggtcatagcccccctccgcctgccagacccacagatcttgaagagtgatacaccttgtgatgacttcactaggtgtcctacaaacaatacctgctgcaaactcaattctggggactggggctgctgtcccatcccagaggctgtctgctgctcagacaaccagcattgctgccctcagggcttcacatgtctggctcaggggtactgtcagaagggagacacaatggtggctggcctggagaagatacctgcccgccagacaaccccgctccaaattggagatatcggttgtgaccagcataccagctgcccagtagggcaaacctgctgcccaagcctcaagggaagttgggcctgctgccagctgccccatgctgtgtgctgtgaggaccggcagcactgttgcccggccgggtacacctgcaatgtgaaggcgaggacctgtgagaaggatgtcgattttatccagcctcccgtgctcctgaccctcggccctaaggttgggaatgtggagtgtggagaagggcatttctgccatgataaccagacctgttgtaaagacagtgcaggagtctgggcctgctgtccctacctaaagggtgtctgctgtagagatggacgtcactgttgccccggtggcttccactgttcagccaggggaaccaagtgtttgcgaaagaagattcctcgctgggacatgtttttgagggatccggtcccaagaccgctactg

SEQ ID NO: 7 - DNA, 1779 bp, Macaca mulatta
  source 1..1779: mol_type = unassigned DNA
atgtggaccctggtgagctgggtggccttaacagtagggctggtggctggaacatggtgcccagatggtcagttctgccctgtggcctgctgcctggaccccggaggagccagctacagctgctgcagtccccttctggacaaatggcccacaacactgagcaggcatctgggcggcccctgccaggctggtgcgcactgctctgctggccactcctgcatcctcaccgtctcagggacttcccgttgctgccccttcccagaggccgtggcgtgcggggatggccatcactgctgcccacggggcttccactgcagtgcagatgggcaatcctgcttccaaagatcaggtaacaactccgtgggtgccgtccagtgccctgatagtcattttgaatgcccggacctctccacgtgctgcgttatggtcgatggctcctgggggtgctgccccatgccccaggcttcctgctgtgaagacagggtgcactgctgtccgcatggtgccttctgcgacctggttcacacccgctgcatcacacccacgggcacccacccactggcaaagaagatccctgcccagaggagtaacagggcagtggctttgtccagctcagtcatgtgtccggatgcacggtcccagtgccctgatggttctacctgctgtgagctgcccagtgggaagtatggctgctgcccaatgcccaatgccatctgctgctccgaccacctgcactgctgcccccaagacactgtgtgtgacctgatccagagtaagtgcctctccaaggagaacactaccatggacctcctcactaagctgcctgcacaaacagtgggagacgtgaaatgtgacatggaggtgagctgcccagatggctatacctgctgccgtctacagtcgggggcctggggctgctgcccttttacccaggctgtgtgctgtgaggaccatatacactgctgtcccgcggggtttacgtgtgacacgcagaagggtacctgtgaacagggaacttaccaggtgtcctggatggagaagaccctagctcacctcagcctgccagacccgcaagccttgaagagagatgtcccctgtgataatgtcaccagctgtcccccctccaatacctgctgcaaactcatgtctggagagtggggctgctgtccagtcccagaggctgtctgctgctcggaccaccagcactgctgcccccagggctacacgtgtgtagccgaggggcggtgtcagcggggaagcgagatcgtggctggactggagaagatgcctgcccgccaggcttccttatcccaccccagagacatgggctgtgaccagcacaccagctgcccagtggggcagacctgctgcccaagcctgggtgggggctgggcctgctgccagttgccccacgctgtgtgctgcgaggatcgccagcactgctgcccggctggctacacctgcaacgtgaaggcccgatcctgcgagaaggaagtggtctctgcccagcctgccaccttcctggcccatagccctcacgtgggtgtgcaggacgtggagtgtgggggagggcacttctgccatgataaccagacctgctgccgagacaaccgagggggctgggcctgctgtccctaccgccagggcatctgttgtgctgatcggcgccactgctgtcctgctggcttccgctgcgcagccaggggcgccaagtgtttgcgcagggaggccctgcgctgggacgcccctttgagggacccagccttgagacagctgctg

SEQ ID NO: 8 - DNA, 1779 bp, synthetic construct
  misc_feature 1..1779: note = Description of Artificial Sequence: Synthetic polynucleotide
  source 1..1779: mol_type = other DNA
atgtggactctcgtatcttgggtcgctctcaccgccggacttgtcgctggaactcgctgtcccgacggacagttctgcccagtggcctgttgcctggaccccgggggcgcgagctacagctgctgccggcctctgctcgacaagtggcctaccacgcttagccggcatctgggcggaccgtgccaggtcgatgcccactgctccgcgggccactcctgcattttcactgtgtccggaacctcctcctgttgcccgttccccgaagccgtggcctgcggcgatggacatcattgctgccctcgcggattccactgctcggcggacgggcgaagctgcttccagcggtccggaaacaactccgtgggagccatccagtgtccggatagccagttcgaatgcccggacttctcaacgtgctgcgtcatggtggacggcagctgggggtgctgccccatgccacaagcctcctgctgtgaagatcgggtgcactgctgtccacacggcgctttttgtgatctggtgcacactcggtgcattactccgaccgggacccacccgctggccaaaaagttgccggcacagcgcaccaacagagccgtggcactcagctcgtccgtgatgtgcccggacgccagatcccggtgccctgacggttcaacttgctgcgagcttccgtcgggaaaatacggctgttgcccaatgcctaacgccacctgttgctcggaccatctgcattgctgtccgcaagacaccgtctgcgacctcatccagtccaagtgcctgagcaaggagaacgccaccaccgacctcctgactaagctgcccgcacacaccgtgggggatgtgaaatgcgacatggaggtgtcctgccccgatggctatacctgttgccgcctgcaatccggagcctggggatgctgccccttcacccaagccgtgtgttgcgaggaccacatccactgctgccccgcgggcttcacatgcgacactcagaagggaacctgtgaacagggtccccatcaagtgccgtggatggaaaaggccccggcccacctgtccttgcctgaccctcaagcgctcaagcgcgacgtgccctgcgacaatgtgtccagctgcccatcctcggacacttgctgtcagctcacctccggggaatggggttgctgcccgatccccgaggcagtgtgctgctcggatcaccaacactgttgcccgcaagggtacacttgtgtggccgagggccagtgccagagaggctccgaaattgtggccggactggagaagatgcctgctcggcgcgcctcactgtcacacccaagggatatcggttgtgaccagcacacctcctgccccgtgggacagacctgttgtccttcactgggtggttcatgggcgtgttgtcagctgccccacgccgtgtgctgcgaggaccggcagcattgttgccccgccgggtacacctgtaacgtgaaggctcggtcctgcgaaaaggaagtggtgtcggcccaaccagccaccttcctggcgcgctcgcctcacgtcggagtcaaggacgtggagtgcggagagggccacttttgccacgacaaccagacttgctgccgggacaaccgccagggatgggcctgctgcccgtaccgccagggcgtttgctgcgctgaccggagacactgctgcccggctggatttcgctgcgccgcccggggaaccaagtgcttgaggagagaagcgccgagatgggatgcacccctgagggacccggcgctgcgccagctgctg

SEQ ID NO: 9 - DNA, 1779 bp, synthetic construct
  misc_feature 1..1779: note = Description of Artificial Sequence: Synthetic polynucleotide
  source 1..1779: mol_type = other DNA
atgtggactctggtctcatgggtcgctctgacagcaggactggtcgcagggacacgatgccccgatggacagttttgccccgtcgcttgctgtctggacccaggaggagccagctactcctgctgtaggccactgctggataagtggcccaccacactgtcccgccacctgggaggaccatgccaggtggacgcacactgttccgccggacactcttgcatcttcacagtgtctggcaccagctcctgctgtccatttcctgaggcagtggcatgcggcgacggacaccactgctgtcccaggggcttccactgtagcgccgatggccggtcctgctttcagagaagcggcaacaattccgtgggcgccatccagtgtcctgacagccagttcgagtgcccagatttttccacctgctgcgtgatggtggatggctcttggggctgctgtccaatgccccaggccagctgctgtgaggacagggtgcactgctgtcctcacggcgccttctgtgatctggtgcacacacgctgcatcacccccacaggcacccaccctctggccaagaagctgccagcacagaggaccaacagggcagtggccctgagcagcagcgtgatgtgcccagacgcacggtctagatgccctgatggcagcacctgctgtgagctgccaagcggcaagtacggctgctgtcctatgccaaacgccacatgctgttccgaccacctgcactgctgtcctcaggacaccgtgtgcgatctgatccagtctaagtgcctgagcaaggagaatgccaccacagacctgctgacaaagctgcctgcccacaccgtgggcgacgtgaagtgtgatatggaggtgtcctgcccagatggctatacatgctgtaggctgcagtctggagcatggggatgctgtcccttcacccaggccgtgtgctgtgaggaccacatccactgctgtcctgccggctttacatgcgatacccagaagggcacatgcgagcagggccctcaccaggtgccatggatggagaaggcaccagcacacctgtccctgcccgaccctcaggccctgaagagagacgtgccttgtgataacgtgtctagctgcccatcctctgatacatgctgtcagctgacctctggcgagtggggctgctgtccaatccccgaggccgtgtgctgtagcgaccaccagcactgctgtcctcagggctatacctgcgtggcagagggacagtgccagaggggctccgagatcgtggcaggcctggagaagatgccagcaaggagagcctctctgagccaccccagagacatcggctgtgatcagcacacaagctgcccagtgggacagacctgctgtccctccctgggaggctcttgggcatgctgtcagctgcctcacgccgtgtgctgtgaggatcggcagcactgctgtccagccggctacacatgcaatgtgaaggccagatcctgcgagaaggaggtggtgtctgcccagccagccaccttcctggcaaggagccctcacgtgggagtgaaggacgtggagtgtggcgagggccacttttgccacgacaaccagacatgctgtcgggataatagacagggctgggcctgctgtccatataggcagggcgtgtgctgtgcagataggcgccactgctgtccagcaggctttaggtgcgcagccagaggcaccaagtgcctgcggagagaagccccccggtgggacgcccccctgcgagaccccgccctgagacagctgctg

SEQ ID NO: 10 - DNA, 1779 bp, synthetic construct
  misc_feature 1..1779: note = Description of Artificial Sequence: Synthetic polynucleotide
  source 1..1779: mol_type = other DNA
atgtggacacttgtgtcttgggttgccctgaccgccggtctggttgcggggacccggtgccctgacggacaattctgccccgtagcttgttgtcttgaccctggtggggcctcatatagctgttgtcgccctctgctcgataaatggcccacaacacttagtaggcacctggggggaccttgccaggtcgacgcccactgttctgccgggcactcctgcatttttactgtaagcggcactagctcttgttgtccttttcccgaagccgtcgcatgcggagatggccaccattgttgcccccgaggattccattgttccgctgatgggaggagctgctttcagcgatctggcaacaatagtgtaggcgctatacaatgtcccgatagccaatttgaatgccctgactttagtacatgctgcgttatggtggacggcagctggggctgttgtcctatgccacaggcctcatgctgtgaggatcgagtacattgctgcccccatggagccttctgtgatttggttcacaccagatgcattacacctacaggaacccaccctctggcaaaaaaactgccagctcagcggaccaatagggctgtggccttgagtagctcagtgatgtgtcctgacgctaggagcaggtgtcctgatggctcaacgtgctgcgaactccccagtggaaagtatggatgctgcccaatgccaaatgctacctgttgtagtgatcacctccattgctgcccccaagacactgtgtgcgacctcatccagtctaagtgcctcagcaaagaaaacgccaccaccgatctgctcacaaagttgcccgctcacacagtcggtgatgtcaaatgtgacatggaggtctcttgcccagatgggtatacttgctgcaggctccagagcggtgcctggggatgttgccctttcactcaagcggtctgctgtgaggaccacatccattgttgcccagccggattcacctgcgacacacaaaaaggtacttgcgagcagggccctcatcaagtgccatggatggaaaaagcccccgcccatcttagcctgccagaccctcaggcacttaagagagacgttccttgcgataatgtctccagttgtccttcctccgacacgtgctgtcagctcactagcggcgaatgggggtgctgccccatacctgaggctgtttgttgttctgaccaccagcactgctgcccccagggctatacatgcgttgcagaaggccagtgtcagcgcggatcagagatcgtggctggactggaaaagatgcccgctagacgcgccagcctcagccacccacgagacatagggtgcgatcaacacacatcctgccctgtgggccagacctgttgccctagcctgggagggtcatgggcctgctgccagctgcctcatgcagtgtgctgtgaagatagacagcactgttgtcccgccggctatacatgtaacgtgaaagcccgatcctgcgagaaagaagtcgtctcagcacagcctgctacatttctcgccaggtctcctcatgtgggtgtgaaggatgtcgaatgtggggaaggccacttttgccacgataatcagacctgttgccgcgataaccgccaggggtgggcgtgttgcccctatagacagggagtgtgttgtgctgatcgaagacactgctgccccgccggattccgatgcgctgcaagaggaacgaaatgcctgcgacgagaagcacccagatgggacgcccctctcagagatcccgccctgcgccagctcctc

SEQ ID NO: 11 - DNA, 1779 bp, synthetic construct
  misc_feature 1..1779: note = Description of Artificial Sequence: Synthetic polynucleotide
  source 1..1779: mol_type = other DNA
atgtggactttggtgtcttgggttgccctgaccgcagggcttgtcgctgggaccaggtgccctgacggacagttttgccccgtagcttgctgtcttgatcctggtggggcatcttatagctgttgcaggcccctgttggataaatggcctactaccctctctaggcacctgggcgggccatgccaggttgatgcacactgttctgccgggcattcctgtatattcactgttagtggaacttcatcctgttgtccttttcctgaagccgtggcctgtggcgatggccatcactgttgccccagaggattccattgttccgccgacggaaggagctgctttcaaaggtcagggaacaacagcgtaggggccatccagtgtcctgactcccaatttgagtgtcccgacttttcaacatgctgtgtcatggtggacggtagctggggctgttgtcctatgcctcaggcgagctgttgcgaggaccgagtgcactgctgcccccatggagcattttgtgatcttgtacacaccagatgcattacccccacaggcacacacccattggcgaaaaaattgcccgcccagcggaccaatagggctgtggccctttcaagctcagtgatgtgccctgacgctaggagtcggtgtcccgatggcagcacatgctgtgagctgccttccgggaagtatggttgctgtccgatgccaaacgctacctgctgctccgatcatctccattgctgtcctcaagatacagtgtgcgatctcattcagagcaagtgcttgtctaaggaaaacgcaactactgatctccttactaagttgccagcccataccgtcggagatgtgaagtgcgatatggaggtttcttgcccagatgggtacacttgctgcagactccagagtggtgcctggggctgctgtccgtttacccaagctgtgtgttgtgaagaccatatccactgctgcccagctggcttcacatgtgacacacagaaaggcacttgcgagcagggcccacaccaggtgccatggatggaaaaggcgcctgctcaccttagcctgccagatccgcaggcacttaagagggatgttccctgtgacaatgtttccagttgtccctcctctgatacatgttgtcagctgacaagcggcgagtgggggtgttgccccattccagaggcggtgtgttgctccgaccaccagcattgttgcccccagggctacacatgcgtggcagagggccagtgtcagcgcgggtctgagattgtagccgggctggaaaagatgcccgccagacgagcttccctgtctcacccaagggatattgggtgtgatcaacacacgtcctgcccggtgggccagacctgttgtccaagtttgggggggtcttgggcttgctgccaactcccacatgcagtgtgctgtgaagaccggcagcactgttgtccggccgggtatacttgcaacgtgaaagctaggtcctgtgaaaaggaagtagtgtcagcccagccagcgactttcctcgctcgaagcccacacgttggtgtgaaagatgtggagtgtggggagggccacttctgtcatgataatcagacctgttgtcgcgataaccgccaagggtgggcgtgttgcccctatcgacagggagtgtgctgtgccgaccgacgccattgttgccccgcgggttttcgctgcgctgcacggggaaccaagtgcctgcggagggaagcccccagatgggacgcccctctccgcgatcccgccctgcgccagctgctg

SEQ ID NO: 12 - DNA, 1779 bp, synthetic construct
  misc_feature 1..1779: note = Description of Artificial Sequence: Synthetic polynucleotide
  source 1..1779: mol_type = other DNA
atgtggactcttgtgtcgtgggtcgctttgactgctggacttgttgctggaacccgatgcccggatggccagttctgcccggtggcctgctgtctggacccgggtggagcctcctactcctgctgccggcccctgcttgacaagtggcccaccaccctgtcccggcacttgggtggaccgtgccaggtggacgcacactgctcagcgggccacagctgcatcttcactgtgtcggggacctcctcatgctgcccattccctgaggcagtggcctgtggggacggccaccactgctgcccccggggcttccactgctcggccgatggcagaagctgcttccagcggtcgggaaacaactcagtgggagccattcagtgccctgactcccaatttgaatgcccggacttcagcacttgctgcgtcatggtggacggttcctggggttgctgtcccatgccccaagcctcctgttgcgaggacagagtccactgctgtcctcacggagccttctgtgacttggtccacaccagatgcatcactcccactgggacccaccccctggccaagaaactgccagcccagcgcaccaacagggcggtggccctgagcagctcagtgatgtgccctgacgcaagatccagatgccccgatggttccacgtgctgcgagctccccagcggaaaatatggctgctgtcctatgcccaatgccacctgttgttcggaccatctgcactgttgcccgcaagacaccgtgtgtgacctgatccagtccaagtgcctcagcaaggagaacgccaccactgatctgctgaccaagctgcctgcacacactgtgggagatgtgaagtgtgacatggaagtgtcctgccctgatggttacacttgctgccggctccagtctggagcgtgggggtgctgtccattcacccaagcagtgtgctgtgaagatcatatccattgctgccctgctggctttacttgtgacacccagaagggaacctgtgaacagggcccgcaccaagtcccttggatggaaaaggccccggcccacctgtccctcccggacccacaggccctcaagagggatgtcccctgtgacaatgtcagctcctgcccttcctcggatacctgttgccaactgacctcgggggaatggggctgctgcccgatccctgaagctgtgtgctgcagtgatcaccagcattgttgcccccaaggctacacctgtgtggcggaagggcagtgccagaggggctcagagattgtggcgggcctggagaagatgcctgccaggagagccagcctctctcatccccgggacattggttgtgatcaacacacctcctgccctgtgggacagacctgttgcccctcactgggaggttcctgggcctgttgccagctgccccatgccgtgtgctgcgaggaccgccagcactgttgtcctgcggggtacacctgtaacgtcaaggcccgcagctgcgaaaaggaagtggtgtcagcacagccagccaccttcctggcccggtccccccacgtgggagtcaaggatgtggagtgcggagagggccatttctgccatgacaaccagacttgctgcagggacaaccgccagggctgggcctgctgcccgtaccgccagggagtgtgctgcgcggacagacgccattgctgtccggctggattccgctgtgctgcccgggggacaaagtgcctgcggcgggaagcccctagatgggatgccccactgagggaccctgccctgcgccagctcctc

SEQ ID NO: 13 - DNA, 1779 bp, synthetic construct
  misc_feature 1..1779: note = Description of Artificial Sequence: Synthetic polynucleotide
  source 1..1779: mol_type = other DNA
atgtggaccttggtatcctgggtcgccctgactgctggacttgtcgctggtaccagatgcccagatggccagttctgtcctgtggcatgctgcctggacccgggaggggcctcctactcctgctgccgccccttgctggacaagtggcccaccaccctgtcccggcatctgggaggaccgtgccaagtggatgcccactgtagcgctggtcacagctgcatcttcactgtgtcgggcactagctcgtgctgcccgttccccgaagcggtggcctgtggagatggacaccactgttgtccccggggattccactgctccgctgatgggcgctcatgcttccaaagatcaggaaacaactctgtgggggccatccagtgcccggactcacagttcgaatgccctgacttctccacgtgctgtgtgatggtggatgggagctggggctgctgccctatgccccaagcctcctgctgtgaagatcgggtgcactgttgcccccatggtgcattttgtgacctggtgcacacccggtgcatcacccctactggaacccacccactggccaagaagctcccggcccaaagaaccaacagggctgtggccctgtcctcctccgtcatgtgcccggatgcccggagcagatgccctgatggcagcacatgctgcgaactgccttcgggaaaatacggttgttgtcccatgccgaacgccacttgctgcagcgaccacctccattgctgtccacaagacactgtgtgcgacctcatccagtccaagtgcctgtccaaggaaaatgccaccacagatctgcttaccaagctgccggcccacactgtgggggatgtgaaatgcgacatggaggtgtcctgcccggatggctacacctgttgccggctgcagtcgggagcctggggctgttgcccattcactcaagcggtctgctgcgaggaccacattcattgttgccctgccggattcacctgtgacacccaaaagggcacttgcgagcagggaccccatcaggtgccctggatggagaaggcccctgcccacctgagcttgccagaccctcaagccctgaagagggatgtcccctgcgacaacgtgtcctcatgcccgtccagcgacacctgttgtcagctcacctccggagaatgggggtgctgccccatccctgaggccgtctgctgctcggaccatcagcactgctgcccccaaggctacacttgcgtggcagagggccagtgtcagcggggatcggaaattgtggcggggctggagaagatgccagcgcgcagggcctcactgagccaccctagggacattggatgtgaccagcacacctcatgccctgtgggccagacctgttgcccttccctgggtggttcctgggcgtgctgccagctccctcatgctgtgtgttgcgaggacagacagcattgctgccccgccggttacacttgcaatgtgaaggctcgatcctgtgaaaaggaggttgtcagtgcccagcctgccaccttcctggcccgctcccctcatgtgggcgtgaaggatgtggaatgcggggaaggccacttttgccacgacaaccagacttgctgccgggacaaccgccagggatgggcctgctgcccgtaccgccagggggtctgctgtgcggacaggcgccactgctgtccggcgggctttagatgcgcagcacggggcaccaagtgcctccggagagaggccccccgctgggatgcacccctgagggacccggccctgagacagcttctc

SEQ ID NO: 14 - DNA, 1770 bp, synthetic construct
  misc_feature 1..1770: note = Description of Artificial Sequence: Synthetic polynucleotide
  source 1..1770: mol_type = other DNA
atgtggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgaga

SEQ ID NO: 15 - DNA, 27 bp, Influenza virus
  source 1..27: mol_type = unassigned DNA
tacccatatgacgtgccggactacgcc

SEQ ID NO: 16 - DNA, 592 bp, Woodchuck hepatitis virus
  source 1..592: mol_type = unassigned DNA
taatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcct

SEQ ID NO: 17 - DNA, 233 bp, Simian virus 40
  source 1..233: mol_type = unassigned DNA
ttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatc

SEQ ID NO: 18 - DNA, 364 bp, Mus musculus
  source 1..364: mol_type = unassigned DNA
acttgtggactaagtttgttcgcatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgcgaggctgtgagcagccacagtgccctgctcagaagccccaagctcgtcagtcaagccggttctccgtttgcactcaggagcacgggcaggcgagtggcccctagttctgggggcagc

SEQ ID NO: 19 - DNA, 1140 bp, Rattus sp.
  source 1..1140: mol_type = unassigned DNA
gtatccagactcactcctttctccttacagtttctgaattccgtattagaagggatggctcatttctagggacaaaataacacaggctctggggtgaggtagggtcgggtaagggggcgagcggggaggggtgatggaagagcactaccgcataaagaaagcaagctctctagtaaagcgttaagtactactttgtatattgtttctcttttttcctttctctctctctctctctttgtttgtttttggtttttgagacagaatatcacactatatatccaggccgggtacaaaataaccgcagtcctccgcttcccaatgctgggatttacaggcataaaccactaagggcggttatgatgaccttgagctgcaagtcttcctgcctctgcctcccaggtgctgttgagggtcataggcgtggtctattcatactgagcttctgaatttgctcaaatattaataataatagtaataataataataaccacaataatacaactgtaaaactaaacatttacccacgcctttttgaccatcattcccatagctcttgctactttatttaaagcgaacagagatgttgaatcctgacggaacgtatttaaatttagtgtagtataaatgaaaagctggaatttaccataaagaatctcaacacaaattctgtgattaagtgttggggaaaaccttaaattatcctaactacagtttaaggtttaagctccgtataatcacccaacccccgttttctttcttccctctctacccctccccagctccaccccataatggatgctcggctagttgcttttgcgaggcctttgtctgaaggatgcaaaatctacggatgctagcgagggggggaaggggggagagattacctcataccatgtcgcttgcaccaatcaccactcctgtcgcggcttctctgggcagacggaggggtctggaccaacaggaaaaggccttggcccatccccatggtgaccgagctgtatataaggaggcgcatccgcccaagtgccggcaggttctcttacatcgaccgcttaagagtcgcgctgtaagaagcaacacctcctcctcgcctccgccatccacccggcagccgcgaagcagcaaccatggtgagcaaac

SEQ ID NO: 20 - DNA, 1801 bp, Rattus sp.
  source 1..1801: mol_type = unassigned DNA
tccttccaaccccctatggtggtatggctgacacagaaaatgtctgctcctgtatgggacatttgcccctcttctccaaatataagacaggatgaggcctagcttttgctgctccaaagttttaaaagaacacattgcacggcatttagggactctaaagggtggaggaggaatgagggaattgcatcatgccaaggctggtcctcatccatcactgcttccagggcccagagtggcttccaggaggtattcttacaaaggaagcccgatctgtagctaacactcagagcccattttcctgcgttaacccctcccgacctcatatacaggagtaacatgatcagtgacctgggggagctggccaaactgcgggacctgcccaagctgagggccttggtgctgctggacaacccctgtgccgatgagactgactaccgccaggaggccctggtgcagatggcacacctagagcgcctagacaaagagtactatgaggacgaggaccgggcagaagctgaggagatccgacagaggctgaaggaggaacaggagcaagaactcgacccggaccaagacatggaaccgtacctcccgccaacttagtggcacctctagcctgcagggacagtaaaggtgatggcaggaaggcagcccccggaggtcaaaggctgggcacgcgggaggagaggccagagtcagaggctgcgggtatctcagatatgaaggaaagatgagagaggctcaggaagaggtaagaaaagacacaagagaccagagaagggagaagaattagagagggaggcagaggaccgctgtctctacagacatagctggtagagactgggaggaagggatgaaccctgagcgcatgaagggaaggaggtggctggtggtatatggaggatgtagctgggccagggaaaagatcctgcactaaaaatctgaagctaaaaataacaggacacggggtggagaggcgaaaggagggcagagtgaggcagagagactgagaggcctggggatgtgggcattccggtagggcacacagttcacttgtcttctctttttccaggaggccaaagatgctgacctcaagaactcataataccccagtggggaccaccgcattcatagccctgttacaagaagtgggagatgttcctttttgtcccagactggaaatccgttacatcccgaggctcaggttctgtggtggtcatctctgtgtggcttgttctgtgggcctacctaaagtcctaagcacagctctcaagcagatccgaggcgactaagatgctagtaggggttgtctggagagaagagccgaggaggtgggctgtgatggatcagttcagctttcaaataaaaaggcgtttttatattctgtgtcgagttcgtgaacccctgtggtgggcttctccatctgtctgggttagtacctgccactatactggaataaggggacgcctgcttccctcgagttggctggacaaggttatgagcatccgtgtacttatggggttgccagcttggtcctggatcgcccgggcccttcccccacccgttcggttccccaccaccacccgcgctcgtacgtgcgtctccgcctgcagctcttgactcatcggggccccccgggtcacatgcgctcgctcggctctataggcgccgccccctgcccaccccccgcccgcgctgggagccgcagccgccgccactcctgctctctctgcgccgccgccgtcaccaccgccaccgccaccggctgagtctgcagtcctcgaggtgaggcccgtatcgg

SEQ ID NO: 21 - DNA, 60 bp, Homo sapiens
  source 1..60: mol_type = unassigned DNA
aaaaaaaaaaaaaaagcccaccctccagcctcgctgcaaagagaaaaccggagcagccgc

SEQ ID NO: 22 - DNA, 806 bp, synthetic construct
  misc_feature 1..806: note = Description of Artificial Sequence: Synthetic polynucleotide
  source 1..806: mol_type = other DNA
gagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactgacatgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacatacataaggtcaataggggtgactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcttctgccttctccctcctgtgagtttggtaagtcactgactgtctatgcctgggaaagggtgggcaggaggtggggcagtgcaggaaaagtggcactgtgaaccctgcagccctagacaattgtactaaccttcttctctttcctctcctgacag

SEQ ID NO: 23 - DNA, 352 bp, synthetic construct
  misc_feature 1..352: note = Description of Artificial Sequence: Synthetic polynucleotide
  source 1..352: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatc

SEQ ID NO: 24 - DNA, 60 bp, Homo sapiens
  source 1..60: mol_type = unassigned DNA
tctcgcactctcccttctcctttataaaggccggaacagctgaaagggtggcaacttctc

SEQ ID NO: 25 - DNA, 60 bp, Homo sapiens
  source 1..60: mol_type = unassigned DNA
gccgcgtccacctgtcggccgggcccagccgagcgcgcagcgggcacgccgcgcgcgcgg

SEQ ID NO: 26 - DNA, 106 bp, Adeno-associated virus
  source 1..106: mol_type = unassigned DNA
gacgcgcgagcgagcgagtgactccggcgggcccgtttcgggcccgcagcccgctggaaaccagcgggccggagtcactcgctcgctcgcgcgtctctccctcacc

SEQ ID NO: 27 - protein, 5 aa, unidentified
  REGION 1..5: note = Description of Unknown: PGRN C-terminal motif sequence
LRQLL

(19) United States
(12) Patent Application Publication
(10) Pub. No.: US 2024/0318199 A1
(43) Pub. Date: Sep. 26, 2024

(54) ADENO-ASSOCIATED VIRUS (AAV) SYSTEMS FOR TREATMENT OF PROGRANULIN ASSOCIATED NEURODEGENERATIVE DISEASES OR DISORDERS

(71) Applicant: Beacon Therapeutics Limited, London (GB)

(72) Inventors: Juan Li, Alachua, FL (US); Tanaz Farivar, Alachua, FL (US); Savitri Mandapati, Alachua, FL (US); Steven Pennock, Leominster, MA (US); Mark Shearman, Alachua, FL (US); Judith Newmark, Milford, MA (US); Adrian M. Timmers, Burlington, MA (US)

(21) Appl. No.: 18/372,868

(22) Filed: Sep. 26, 2023

Related U.S. Application Data
(63) Continuation of application No. 17/077,680, filed on Oct. 22, 2020, now abandoned.
(60) Provisional application No. 62/924,340, filed on Oct. 22, 2019.

(51) Int. Cl. C12N 15/86; C07K 14/47

(52) U.S. Cl. CPC C12N 15/86; C07K 14/47; C12N 2750/14141

(57) ABSTRACT

The disclosure provides, in part, optimally-modified progranulin (PGRN) cDNA and associated genetic elements for use in recombinant adeno-associated virus (rAAV)-based gene therapy for neurodegenerative disorders characterized by cognitive disruption, behavioral impairment, and deficient lysosomal storage, including familial frontotemporal dementia (FTD), frontotemporal lobar degeneration (FTLD), neuronal ceroid lipofuscinosis (NCL), or Alzheimer's disease (AD).

### CROSS-REFERENCE TO RELATED APPLICATIONS

This application is a Continuation of U.S. patent application Ser. No. 17/077,680 filed on Oct. 22, 2020, which claims benefit under 35 U.S.C. § 119 (c) of U.S. Provisional Application No. 62/924,340 filed Oct. 22, 2019, the contents of which is incorporated herein by reference in its entirety.

### SEQUENCE LISTING INCORPORATION PARAGRAPH

The instant application contains a Sequence Listing which has been submitted electronically in XML file format and is hereby incorporated by reference in its entirety. Said XML copy, created on Jun. 14, 2024, is named 119561-01803_SL.xml and is 54,238 bytes in size.

### FIELD OF THE INVENTION

The present invention relates to the field of gene therapy, including AAV vectors for expressing an isolated polynucleotides in a subject or cell. The disclosure also relates to nucleic acid constructs, promoters, vectors, and host cells including the polynucleotides as well as methods of delivering exogenous DNA sequences to a target cell, tissue, organ or organism, and methods for use in the treatment or prevention of progranulin associated neurodegenerative diseases or disorders.

### BACKGROUND OF THE INVENTION

Gene therapy aims to improve clinical outcomes for patients suffering from either genetic mutations or acquired diseases caused by an aberration in the gene expression profile. Gene therapy includes the treatment or prevention of medical conditions resulting from defective genes or abnormal regulation or expression, e.g. underexpression or overexpression, that can result in a disorder, disease, malignancy, etc. For example, a disease or disorder caused by a defective gene might be treated, prevented or ameliorated by delivery of a corrective genetic material to a patient, or might be treated, prevented or ameliorated by altering or silencing a defective gene, e.g., with a corrective genetic material to a patient resulting in the therapeutic expression of the genetic material within the patient.

The basis of gene therapy is to supply a transcription cassette with an active gene product (sometimes referred to as a transgene or a therapeutic nucleic acid), e.g., that can result in a positive gain-of-function effect, a negative loss-of-function effect, or another outcome. Such outcomes can be attributed to expression of a therapeutic protein such as an antibody, a functional enzyme, or a fusion protein. Gene therapy can also be used to treat a disease or malignancy caused by other factors. Human monogenic disorders can be treated by the delivery and expression of a normal gene to the target cells. Delivery and expression of a corrective gene in the patient's target cells can be carried out via numerous methods, including the use of engineered viruses and viral gene delivery vectors.

Adeno-associated viruses (AAV) belong to the Parvoviridae family and more specifically constitute the dependoparvovirus genus. Vectors derived from AAV (i.e., recombinant AAV (rAVV) or AAV vectors) are attractive for delivering genetic material because (i) they are able to infect (transduce) a wide variety of non-dividing and dividing cell types including myocytes and neurons; (ii) they are devoid of the virus structural genes, thereby diminishing the host cell responses to virus infection, e.g., interferon-mediated responses; (iii) wild-type viruses are considered non-pathologic in humans; (iv) in contrast to wild type AAV, which are capable of integrating into the host cell genome, replication-deficient AAV vectors lack the rep gene and generally persist as episomes, thus limiting the risk of insertional mutagenesis or genotoxicity; and (v) in comparison to other vector systems, AAV vectors are generally considered to be relatively poor immunogens and therefore do not trigger a significant immune response (see ii), thus gaining persistence of the vector DNA and potentially, long-term expression of the therapeutic transgenes.

Progranulin (PGRN) is a widely expressed, secreted glycoprotein that acts as a trophic factor for many cell types, including neuronal cells, modulates inflammation, and facilitates wound repair. PGRN is involved in the regulation of multiple processes including development, wound healing, angiogenesis, growth and maintenance of neuronal cells, and inflammation. PGRN is expressed in neurons and microglia (Petkau et al., Neurol. 2010. 518, 3931-3947) and has been implicated in inflammation (Yin et al., J. Exp. Med. 2010. 207, 117-12; Tang et al., Science. 2010; 332, 478-484), wound repair (He et al., Nat. Med. 2003. 9. 225-229) and neurite outgrowth (Van Damme et al., J. Cell Biol. 181, 37-41). In microglia, progranulin is constitutively expressed and secreted. Progranulin in neurons is important for proper trafficking and function of lysosomal enzymes such as β-glucocerebrosidase and cathepsin D.

Sortilin binds PGRN and targets it for lysosomal degradation, thus negatively regulating extracellular levels of PGRN (Hu, F et al. 2010. Neuron 68, 654-667). In line with this, deficiency of Sortilin significantly increases plasma PGRN levels both in mouse models in vivo and human cells In vitro (Carrasquillo. M. M et al., 2010. Am J Hum Genet 87, 890-897; Lee, W. C et al., 2010. 23, 1467-1478). A polymorphism in Sortilin was shown to be strongly associated with PGRN serum levies in humans (Carrasquillo M. et al., 2010. Am J Hum Genet. 10; 87 (6): 890-7). Tanaka et al., Hum Mol Genet. 2017 Mar. 1; 26 (5): 969-988; Paushter et al., Acta Neuropathol. 2018 July; 136 (1): 1-1; Hu et al., Neuron. 2010 Nov. 18; 68 (4): 654-67; Zheng et al., PLOS One. 2011. 6 (6): e21023; Nicholson et al., Nat Commun. 2016 Jun. 30; 7:11992; Zhou et al., J Neurochem. 2017 October; 143 (2): 236-243).

Altered PGRN expression has been shown in multiple neurodegenerative disorders, and recent studies into the genetic etiology of neurodegenerative diseases have shown that heritable mutations in the PGRN gene may lead to adult-onset neurodegenerative disorders due to reduced neuronal survival. Complete PGRN deficiency (i.e., homozygous PGRN mutants) and loss-of-function mutations lead to the neurodegenerative diseases or disorders, including but not limited to familial frontotemporal dementia (FTD), and the neurodegenerative lysosomal storage disorder neuronal ceroid lipofuscinosis (NCL), including neuronal ceroid lipofuscinosis 11 (CLN11). Frontotemporal dementia (FTD) encompasses a group of neurodegenerative disorders characterized by cognitive and behavioral impairments. Heterozygous mutations in progranulin (PGRN) cause familial FTD and result in decreased PGRN expression, while homozygous mutations result in complete loss of PGRN expression and lead to NCL. CLN11, also termed adult-onset CLN, shares some clinical features with FTD such as cognitive decline and eventual fatality, but is also characterized by progressive visual loss via retinal dystrophy, seizures, cerebellar ataxia, and cerebellar atrophy. Low PGRN promotes neuroinflammation and enhances peripheral inflammatory conditions such as arthritis and atherosclerosis, and thus any disorder characterized by neuroinflammation or peripheral inflammation could be a potential target for PGRN. In particular, low PGRN is risk factor for schizophrenia, bipolar and psychiatric disorders (Chitramuthu et al., Brain. 2017 Dec. 1; 140 (12): 3081-3104).

FTD is a fatal, degenerative brain disease that is a common cause of dementia in people under the age of 60. Often striking people in their prime. FTD is characterized by a progressive degeneration of the frontal portions of the brain, the regions responsible for language and behavior. Over the course of the disease, FTD patients may lose the ability to behave appropriately, make judgements, communicate, and carry out daily activities. Frontotemporal lobar degeneration (FTLD) refers to a collection of pathologic diagnoses that can cause FTD syndromes. Progranulin supplementation may be used to treat multiple disease conditions including neurodegenerative diseases such as Alzheimer's disease (AD), Parkinson's disease (PD) and Parkinson's-like diseases (Capell et al., 2011; Cenik et al., 2011; Van Kampen et al., 2014; Minami et al., 2015), acute brain injury (Tao et al., 2012; Egashira et al., 2013; Jackman et al., 2013; Kanazawa et al., 2015; Zhao and Bateman, 2015; Altmann et al., 2016b; Xie et al., 2016). Furthermore, progranulin has been suggested as a therapeutic target for many peripheral conditions particularly those with an important inflammatory component (He et al., 2003; Sfikakis and Tsokos, 2011; Guo et al., 2012; Jian et al., 2013; Choi et al., 2014; Huang et al., 2015; Zhou et al., 2015a). Heterozygous mutations in PGRN) leading to haplo-insufficiency, are responsible for approximately 20% of familial FTD (The Association for Frontotemporal Degeneration website (2019); Petkau & Leavitt Trends in Neurosci. 2014 37 (7): 388-98).

Neurodegenerative disorders represent a considerable social and economic challenge in an aging society. There are currently no approved treatments or cures for FTD, even though FTD is second only to Alzheimer's in terms of prevalence and incidence on the dementia spectrum. Current treatment of neurodegenerative disorders generally involves efforts by physicians to slow progression of the symptoms and make patients more comfortable, and most treatments only mask the progression of neurologic decline. Currently, there are no progranulin gene therapies in clinical development for neurodegenerative diseases or disorders. Thus, a need remains for methods and compositions for the treatment of neurodegenerative diseases or disorders.

### SUMMARY OF THE INVENTION

The disclosure relates to recombinant adeno-associated virus (rAAV) vectors through which a progranulin expression cassette can be packaged for CNS-targeted delivery to patients suffering from neurodegenerative disorders associated with progranulin (PGRN) mutations.

According to one aspect, the disclosure provides an isolated polynucleotide comprising a nucleic acid sequence encoding progranulin. According to some embodiments, the nucleic acid sequence is a non-naturally occurring sequence. According to some embodiments, the nucleic acid sequence encodes mammalian progranulin. According to some embodiments, the mammalian progranulin is human progranulin. According to some embodiments, the nucleic acid comprises a sequence selected from the group consisting of: SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13 and SEQ ID NO: 14. According to some embodiments, the nucleic acid comprises a sequence that is at least 85% identical to a nucleic acid sequence selected from the group consisting of: SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13 and SEQ ID NO: 14. According to some embodiments, the nucleic acid comprises a sequence that is at least 85% identical to SEQ ID NO: 5. According to some embodiments, the nucleic acid comprises a sequence that is at least 90% identical to SEQ ID NO: 5. According to some embodiments, the nucleic acid comprises a sequence that is at least 95% identical to SEQ ID NO: 5. According to some embodiments, the nucleic acid comprises a sequence that is at least 96% identical to SEQ ID NO: 5. According to some embodiments, the nucleic acid comprises a sequence that is at least 97% identical to SEQ ID NO: 5. According to some embodiments, the nucleic acid comprises a sequence that is at least 98% identical to SEQ ID NO: 5. According to some embodiments, the nucleic acid comprises a sequence that is at least 99% identical to SEQ ID NO: 5. According to some embodiments, the nucleic acid consists of SEQ ID NO: 5. According to some embodiments, the nucleic acid comprises a sequence that is at least 85% identical to SEQ ID NO: 6. According to some embodiments, the nucleic acid comprises a sequence that is at least 90% identical to SEQ ID NO: 6. According to some embodiments, the nucleic acid comprises a sequence that is at least 95% identical to SEQ ID NO: 6. According to some embodiments, the nucleic acid comprises a sequence that is at least 96% identical to SEQ ID NO: 6. According to some embodiments, the nucleic acid comprises a sequence that is at least 97% identical to SEQ ID NO: 6. According to some embodiments, the nucleic acid comprises a sequence that is at least 98% identical to SEQ ID NO: 6. According to some embodiments, the nucleic acid comprises a sequence that is at least 99% identical to SEQ ID NO: 6. According to some embodiments, the nucleic acid consists of SEQ ID NO: 6. According to some embodiments, the nucleic acid comprises a sequence that is at least 85% identical to SEQ ID NO: 7. According to some embodiments, the nucleic acid comprises a sequence that is at least 90% identical to SEQ ID NO: 7. According to some embodiments, the nucleic acid comprises a sequence that is at least 95% identical to SEQ ID NO: 7. According to some embodiments, the nucleic acid comprises a sequence that is at least 96% identical to SEQ ID NO: 7. According to some embodiments, the nucleic acid comprises a sequence that is at least 97% identical to SEQ ID NO: 7. According to some embodiments, the nucleic acid comprises a sequence that is at least 98% identical to SEQ ID NO: 7. According to some embodiments, the nucleic acid comprises a sequence that is at least 99% identical to SEQ ID NO: 7. According to some embodiments, the nucleic acid consists of SEQ ID NO: 7. According to some embodiments, the nucleic acid comprises a sequence that is at least 85% identical to SEQ ID NO: 8. According to some embodiments, the nucleic acid comprises a sequence that is at least 90% identical to SEQ ID NO: 8. According to some embodiments, the nucleic acid comprises a sequence that is at least 95% identical to SEQ ID NO: 8. According to some embodiments, the nucleic acid comprises a sequence that is at least 96% identical to SEQ ID NO: 8. According to some embodiments, the nucleic acid comprises a sequence that is at least 97% identical to SEQ ID NO: 8. According to some embodiments, the nucleic acid comprises a sequence that is at least 98% identical to SEQ ID NO: 8. According to some embodiments, the nucleic acid comprises a sequence that is at least 99% identical to SEQ ID NO: 8. According to some embodiments, the nucleic acid consists of SEQ ID NO: 8. According to some embodiments, the nucleic acid comprises a sequence that is at least 85% identical to SEQ ID NO: 9. According to some embodiments, the nucleic acid comprises a sequence that is at least 90% identical to SEQ ID NO: 9. According to some embodiments, the nucleic acid comprises a sequence that is at least 95% identical to SEQ ID NO: 9. According to some embodiments, the nucleic acid comprises a sequence that is at least 96% identical to SEQ ID NO: 9. According to some embodiments, the nucleic acid comprises a sequence that is at least 97% identical to SEQ ID NO: 9. According to some embodiments, the nucleic acid comprises a sequence that is at least 98% identical to SEQ ID NO: 9. According to some embodiments, the nucleic acid comprises a sequence that is at least 99% identical to SEQ ID NO: 9. According to some embodiments, the nucleic acid consists of SEQ ID NO: 9. According to some embodiments, the nucleic acid comprises a sequence that is at least 85% identical to SEQ ID NO: 10. According to some embodiments, the nucleic acid comprises a sequence that is at least 90% identical to SEQ ID NO: 10. According to some embodiments, the nucleic acid comprises a sequence that is at least 95% identical to SEQ ID NO: 10. According to some embodiments, the nucleic acid comprises a sequence that is at least 96% identical to SEQ ID NO: 10. According to some embodiments, the nucleic acid comprises a sequence that is at least 97% identical to SEQ ID NO: 10. According to some embodiments, the nucleic acid comprises a sequence that is at least 98% identical to SEQ ID NO: 10. According to some embodiments, the nucleic acid comprises a sequence that is at least 99% identical to SEQ ID NO: 10. According to some embodiments, the nucleic acid consists of SEQ ID NO: 10. According to some embodiments, the nucleic acid comprises a sequence that is at least 85% identical to SEQ ID NO: 11. According to some embodiments, the nucleic acid comprises a sequence that is at least 90% identical to SEQ ID NO: 11. According to some embodiments, the nucleic acid comprises a sequence that is at least 95% identical to SEQ ID NO: 11. According to some embodiments, the nucleic acid comprises a sequence that is at least 96% identical to SEQ ID NO: 11. According to some embodiments, the nucleic acid comprises a sequence that is at least 97% identical to SEQ ID NO: 11. According to some embodiments, the nucleic acid comprises a sequence that is at least 98% identical to SEQ ID NO: 11. According to some embodiments, the nucleic acid comprises a sequence that is at least 99% identical to SEQ ID NO: 11. According to some embodiments, the nucleic acid consists of SEQ ID NO: 11. According to some embodiments, the nucleic acid comprises a sequence that is at least 85% identical to SEQ ID NO: 12. According to some embodiments, the nucleic acid comprises a sequence that is at least 90% identical to SEQ ID NO: 12. According to some embodiments, the nucleic acid comprises a sequence that is at least 95% identical to SEQ ID NO: 12. According to some embodiments, the nucleic acid comprises a sequence that is at least 96% identical to SEQ ID NO: 12. According to some embodiments, the nucleic acid comprises a sequence that is at least 97% identical to SEQ ID NO: 12. According to some embodiments, the nucleic acid comprises a sequence that is at least 98% identical to SEQ ID NO: 12. According to some embodiments, the nucleic acid comprises a sequence that is at least 99% identical to SEQ ID NO: 12. According to some embodiments, the nucleic acid consists of SEQ ID NO: 12. According to some embodiments, the nucleic acid comprises a sequence that is at least 85% identical to SEQ ID NO: 13. According to some embodiments, the nucleic acid comprises a sequence that is at least 90% identical to SEQ ID NO: 13. According to some embodiments, the nucleic acid comprises a sequence that is at least 95% identical to SEQ ID NO: 13. According to some embodiments, the nucleic acid comprises a sequence that is at least 96% identical to SEQ ID NO: 13. According to some embodiments, the nucleic acid comprises a sequence that is at least 97% identical to SEQ ID NO: 13. According to some embodiments, the nucleic acid comprises a sequence that is at least 98% identical to SEQ ID NO: 13. According to some embodiments, the nucleic acid comprises a sequence that is at least 99% identical to SEQ ID NO: 13. According to some embodiments, the nucleic acid consists of SEQ ID NO: 13. According to some embodiments, the nucleic acid sequence is codon optimized for mammalian expression. According to some embodiments, the nucleic acid sequence is codon optimized for expression in human cells. According to some embodiments, the nucleic acid sequence is a cDNA sequence. According to some embodiments, the nucleic acid sequence further comprises an operably linked functionally optimized N-terminal signal sequence. According to some embodiments, the nucleic acid sequence further comprises an operably linked hemagglutinin C-terminal tag. According to some embodiments, the nucleic acid sequence further comprises an operably linked sortilin binding inhibitory (SBI) domain. According to some embodiments, the nucleic acid sequence further comprises an operably linked neuron-specific human synapsin-1 promoter (hSYN1). According to some embodiments, the nucleic acid sequence further comprises an operably linked ubiquitously-active CBA promoter. According to some embodiments, the nucleic acid sequence further comprises the mouse calcium/calmodulin-dependent protein kinase II (CaMKII) promoter. According to some embodiments, the nucleic acid sequence further comprises the rat tubulin alpha 1 (Ta1) promoter. According to some embodiments, the nucleic acid sequence further comprises the Rat neuron-specific enolase (NSE) promoter. According to some embodiments, the nucleic acid sequence further comprises human platelet-derived growth factor-beta chain (PDGF) promoter. According to some embodiments, the nucleic acid sequence further comprises the EF1alpha promoter. According to some embodiments, any of the promoters described in the aspects and embodiments herein may further comprise an additional CAG/CMV enhancer element, situated 5′ (upstream) of the promoter sequence. According to some embodiments, the promoter is optimized to drive high progranulin expression. According to some embodiments, the nucleic acid sequence further comprises an operably linked 3′UTR regulatory region comprising a Woodchuck Hepatitis Virus Posttranscriptional Regulatory Element (WPRE). According to some embodiments, the nucleic acid sequence further comprises an operably linked polyadenylation signal. According to some embodiments, the polyadenylation signal is an SV40 polyadenylation signal. According to some embodiments, the polyadenylation signal is a human growth hormone (hGH) polyadenylation signal. According to some embodiments, the polynucleotide further comprises an operably linked N-terminal signal sequence, optionally comprising a hemagglutinin C-terminal tag or sortilin binding inhibitory (SBI) domain, operably linked to a neuron-specific human synapsin-1 promoter, mouse calcium/calmodulin-dependent protein kinase II (CaMKII) promoter, rat tubulin alpha 1 (Ta1) promoter, Rat neuron-specific enolase (NSE) promoter, Human platelet-derived growth factor-beta chain (PDGF) promoter, or ubiquitously-active CBA promoter, ubiquitously-active EF1alpha promoter, or any of the promoters set forth in any of the aspects or embodiments herein, further comprising an additional 5′ CAG/CMV enhancer element, operably linked to a 3′UTR regulatory region comprising a Woodchuck Hepatitis Virus Posttranscriptional Regulatory Element (WPRE) operably linked to a polyadenylation signal.

According to some embodiments, the disclosure provides a host cell comprising the polynucleotide of any of the aspects or embodiments herein. According to some embodiments, the host cell is a mammalian cell.

According to some embodiments, the disclosure provides a recombinant herpes simplex virus (rHSV) comprising the polynucleotide of any of the aspects or embodiments herein.

According to some embodiments, the disclosure provides a transgene expression cassette comprising the polynucleotide of any one of the aspects or embodiments herein, and minimal regulatory elements. According to some embodiments, the disclosure provides a nucleic acid vector comprising the expression cassette of claim 24. According to some embodiments, the vector is an adeno-associated viral (AAV) vector. According to some embodiments, the disclosure provides a host cell comprising the transgene expression cassette of any of the aspects or embodiments herein. According to some embodiments, the disclosure provides an expression vector comprising the polynucleotide of any of the aspects and embodiments herein. According to some embodiments, the vector is an adeno-associated vital (AAV) vector. According to some embodiments, the serotype of the capsid sequence and the serotype of the ITRs of said AAV vector are independently selected from the group consisting of AAV1. AAV2, AAV3, AAV4, AAV5, AAV6, AAV7, AAV8, AAV9, AAV10, AAV11, and AAV12.

According to some embodiments, the disclosure provides a recombinant adeno-associated (rAAV) expression vector comprising the polynucleotide of any of the aspects and embodiments herein, and an AAV genomic cassette. According to some embodiments, the AAV genomic cassette is flanked by two sequence-modulated inverted terminal repeats.

According to some embodiments, the disclosure provides a recombinant adeno-associated (rAAV) expression vector comprising the polynucleotide of any one of the aspects and embodiments herein, operably linked to a N-terminal signal sequence, optionally comprising a hemagglutinin C-terminal tag or sortilin binding inhibitory (SBI) domain, operably linked to a neuron-specific human synapsin-1 promoter (hSYN), a mouse calcium/calmodulin-dependent protein kinase II (CaMKII) promoter, a rat tubulin alpha 1 (Ta1) promoter, a rat neuron-specific enolase (NSE) promoter, a human platelet-derived growth factor-beta chain (PDGF) promoter, or ubiquitously-active CBA promoter, a ubiquitously-active EF1alpha promoter, or any of the promoters set forth in any of the aspects or embodiments herein, further comprising an additional 5′ CAG/CMV enhancer element, operably linked to a 3′UTR regulatory region comprising a Woodchuck Hepatitis Virus Posttranscriptional Regulatory Element (WPRE) operably linked to a polyadenylation signal, wherein two sequence modulated inverted terminal repeats (ITRs) flank the AAV genomic cassette, and further comprise a protein capsid variant. According to some embodiments, the polyadenylation signal is a SV40 or human growth hormone (hGH) polyadenylation signal. According to some embodiments, the promoter is optimized to drive high progranulin expression. According to some embodiments, the rAAV is a serotype selected from the group consisting of AAV1, AAV2, AAV3, AAV4, AAV5, AAV6, AAV7, AAV8, AA-9, rhAAV10 (also called AAVrh10), AAV10, AAV11, and AAV12. According to some embodiments, the rAAV is a variant or hybrid of a serotype selected from the group consisting of AAV1, AAV2, AAV3, AAV4, AAV5. AAV6. AAV7. AAV8. AAV9, rh-AAV10, AAV10, AAV11, and AAV12. According to some embodiments, the rAAV is comprised within an AAV virion.

According to some aspects, the disclosure comprises an expression vector comprising AAVrh10-hSYN-PGRNwt.

According to some embodiments, the disclosure provides a recombinant herpes simplex virus (rHSV) comprising the expression vector of any of the aspects or embodiments herein. According to some embodiments, the disclosure provides a host cell comprising the expression vector of any of the aspects and embodiments herein. According to some embodiments, the host cell is a mammalian cell.

According to some embodiments, the disclosure provides a transgene expression cassette comprising a polynucleotide of any of the aspects and embodiments herein, and minimal regulatory elements. According to some embodiments, the disclosure provides a nucleic acid vector comprising the expression cassette of any of the aspects or embodiments herein. According to some embodiments, the vector is an adeno-associated viral (AAV) vector.

According to some embodiments, the disclosure provides a composition comprising a polynucleotide of any of the aspects or embodiments herein. According to some embodiments, the disclosure provides a composition comprising a host cell of any of the aspects or embodiments herein. According to some embodiments, the disclosure provides a composition comprising a recombinant herpes simplex virus (rHSV) of any of the aspects or embodiments herein. According to some embodiments, the disclosure provides a composition comprising a transgene expression cassette of any of the aspects or embodiments herein. According to some embodiments, the disclosure provides a composition comprising an expression vector of any of the aspects or embodiments herein. According to some embodiments, the composition is a pharmaceutical composition.

According to some embodiments, the disclosure provides a method of treating a neurodegenerative disorder comprising administering the polynucleotide of any of the aspects or embodiments herein to a subject in need thereof.

According to some embodiments, the disclosure provides a method of treating a neurodegenerative disorder comprising administering the transgene expression cassette of any of the aspects or embodiment herein to a subject in need thereof.

According to some embodiments, the disclosure provides a method of treating a neurodegenerative disorder comprising administering the expression vector of any of the aspects or embodiments herein to a subject in need thereof.

According to some embodiments, the disclosure provides a method of treating a neurodegenerative disorder comprising administering the recombinant adeno-associated (rAAV) expression vector of any of the aspects or embodiments herein to a subject in need thereof.

According to some embodiments, the disclosure provides a method of preventing a neurodegenerative disorder comprising administering the polynucleotide of any of the aspects or embodiments herein to a subject in need thereof.

According to some embodiments, the disclosure provides a method of preventing a neurodegenerative disorder comprising administering the transgene expression cassette of any of the aspects or embodiment herein to a subject in need thereof.

According to some embodiments, the disclosure provides a method of preventing a neurodegenerative disorder comprising administering the expression vector of any of the aspects or embodiments herein to a subject in need thereof.

According to some embodiments, the disclosure provides a method of preventing a neurodegenerative disorder comprising administering the recombinant adeno-associated (rAAV) expression vector of any of the aspects or embodiments herein to a subject in need thereof.

According to some embodiments, the disclosure provides a method of treating a neurodegenerative disorder comprising administering a recombinant adeno-associated (rAAV) viral particle comprising the polynucleotide of any of the aspects or embodiments herein to a subject in need thereof. According to some embodiments, the disclosure provides a method of preventing a neurodegenerative disorder comprising administering a recombinant adeno-associated (rAAV) viral particle comprising the polynucleotide of any of the aspects or embodiments herein to a subject in need thereof. According to some embodiments, the neurodegenerative disorder is characterized by cognitive disruption, behavioral impairment, deficient lysosomal storage, or combination thereof. According to some embodiments, the neurodegenerative disorder is a progranulin-associated neurodegenerative disorder. According to some embodiments, the neurodegenerative disorder is familial frontotemporal dementia (FTD), frontotemporal lobar degeneration (FTLD), neuronal ceroid lipofuscinosis (NCL), or Alzheimer's disease (AD). According to some embodiments, the neuronal ceroid lipofuscinosis (NCL) is neuronal ceroid lipofuscinosis-11 (CLN11). According to some embodiments, the administration is to the central nervous system. According to some embodiments, the administration is intravenous, intra cerebroventricular, intrathecal, or a combination thereof.

According to one aspect, the disclosure provides a method for producing recombinant AAV viral particles comprising: co-infecting a suspension a cell with a first recombinant herpesvirus comprising a nucleic acid encoding an AAV rep and an AAV cap gene each operably linked to a promoter; and a second recombinant herpesvirus comprising a progranulin gene, and a promoter operably linked to said gene; and allowing the cell to produce the recombinant AAV viral particles, thereby producing the recombinant AAV viral particles. According to some embodiments, the cap gene is selected from an AAV with a serotype selected from the group consisting of AAV-1, AAV-2, AAV-3, AAV-4, AAV-5, AAV-6, AAV-7, AAV-8, AAV-9, rh-AAV-10, AAV11, and AAV12. According to some embodiments, the first herpesvirus and the second herpesvirus are viruses selected from the group consisting of cytomegalovirus (CMV), herpes simplex (HSV) and varicella zoster (VZV) and epstein barr virus (EBV). According to some embodiments, the herpesvirus is replication defective. According to some embodiments, the co-infection is simultaneous.

### BRIEF DESCRIPTION OF THE DRAWINGS

FIG. 1 shows a schematic of a PGRN construct, comprising inverted terminal repeats (ITR) at each end, a human synapsin 1 (hSYN1) or chicken beta actin (CBA) promoter, PGRN optionally comprising a C-terminal HA tag or a deletion of 3-16 nucleic acids in the C-terminal, Woodchuck Hepatitis Virus (WHP) Posttranscriptional Regulatory Element (WPRE), SV40 early polyadenylation signals (SV40 pA) and optionally stuffer DNA. The bottom panel shows a schematic of a self-complementary AAV genome.

FIG. 2 shows the nucleic acid sequence of the chicken beta actin (CBA) promoter (SEQ ID NO: 1).

FIG. 3 shows the nucleic acid sequence of the human synapsin 1 (hSYN1) promoter (SEQ ID NO: 2).

FIG. 4 shows the 5′-3′ inverted terminal repeat (ITR) for single stranded (ss) and self-complimentary (sc) AAV genomes (SEQ ID NO: 3).

FIG. 5A shows the 3′-5′ ITR for single stranded (ss) AAV genomes only′ (SEQ ID NO: 4).

FIG. 5B shows the 3′-5′ TRS for self-complimentary (sc) AAV genomes only (SEQ ID NO: 26). TRS refers to a shortened ITR.

FIG. 6 shows the nucleic acid sequence of wild type human progranulin (hPGRNwt) (SEQ ID NO: 5).

FIG. 7 shows the nucleic acid sequence of wild type mouse progranulin (mPGRNwt) (SEQ ID NO: 6). mPGRNwt is 78% similar to hPGRN.

FIG. 8 shows the nucleic acid sequence of macaque (Macaca mulatta) (SEQ ID NO: 7).

Macaque PGRN is 96% similar to hPGRN.

FIG. 9 shows the nucleic acid sequence of hPGRNcoA (SEQ ID NO: 8).

FIG. 10 shows the nucleic acid sequence of hPGRNcoB (SEQ ID NO: 9).

FIG. 11 shows the nucleic acid sequence of hPGRNcoC (SEQ ID NO: 10).

FIG. 12 shows the nucleic acid sequence of hPGRNcoD (SEQ ID NO: 11).

FIG. 13 shows the nucleic acid sequence of hPGRNcoE (SEQ ID NO: 12).

FIG. 14 shows the nucleic acid sequence of hPGRNcoF (SEQ ID NO: 13).

FIG. 15 shows the nucleic acid sequence of a hPGRN sortilin-binding deficient variant (SEQ ID NO: 14).

FIG. 16 shows the nucleic acid sequence of a hemagglutinin (HA) tag (SEQ ID NO: 15).

FIG. 17 shows the nucleic acid sequence of WPRE (SEQ ID NO: 16).

FIG. 18 shows the nucleic acid sequence of SV40 pA (SEQ ID NO: 17).

FIG. 19 shows the nucleic acid sequence of CaMKII promoter (364 bp) corresponding to Sequence from GenBank AJ222796, nuc 7625-7988 (SEQ ID NO: 18).

FIG. 20 shows the nucleic acid sequence of Rat tubulin alpha 1 (Ta1) promoter (1034 bp) (SEQ ID NO: 19).

FIG. 21 shows the nucleic acid sequence of Rat neuron-specific enolase (NSE) promoter (1801 bp) (SEQ ID NO: 20).

FIG. 22 shows the nucleic acid sequence of a human platelet-derived growth factor-beta chain (PDGF-B) promoter (SEQ ID NO: 21 (PDGFB_1), SEQ ID NO: 24 (PDG-B_2) and SEQ ID NO: 25 (PDGFB_3)). There are three defined PDGF-B promoters in human genome, spanning location 39244982-39244621 on the [-] strand of chromosome NC_000022.11. The sequence for the PDGF-B promoter includes at minimum 1 of the 3 core promoter sequences provided or any unique combination thereof. Interspersed sequences can be any non-coding, non-regulatory functional sequence, including no sequence at all.

FIG. 23 shows the nucleic acid sequence of EF1alpha promoter (806 bp) (SEQ ID NO: 22).

FIG. 24 shows the nucleic acid sequence of CAG/CMV Enhancer (SEQ ID NO: 23).

FIG. 25 summarizes results of rAAVrh10, AAV9, ΔHSmax and AAV2tyf capsids that were tested for expression of GFP reporter in non-human primates after intrathecal injection. Percent GFP positive cells and GFP intensity in various regions of the brain are shown.

FIG. 26 summarizes the results of rAAVrh10 biodistribution in non-human primates by injection into the cisterna magna (ICM).

FIG. 27 shows graphs that depict the results of experiments testing the effects of CBA or hSYN promoters on GFP expression in HEK293 and SH-SY5Y cells.

FIG. 28 shows graphs that depict GFP expression after rAAVRh10-CBA-hGFP transduction in HEK293 and SHSY-5Y cells, with and without AD5 vector.

FIG. 29 shows the results of an ELISA experiment to determine protein expression of the 6 PGRN codon optimized variants (designated coA-coF) compared to wild type.

FIG. 30 shows the results of a Western blot experiment to determine protein expression of the 6 PGRN codon optimized variants (designated coA-coF) compared to wild type.

FIG. 31 shows a graph that depicts the results of testing the effects of the codon optimized variants coE (PGRNcoE) and coF (PGRNcoF) under the control of hSYN promoter on progranulin expression in HEK 293 cells.

FIG. 32 shows the results of a Western blot experiment to confirm progranulin expression.

FIG. 33 shows the results of an ELISA experiment to determine protein expression of the 6 PGRN codon optimized variants (designated coA-coF) compared to wild type.

FIG. 34 shows progranulin expression determined after 8 and 10 weeks in an in vivo study in NHP with rAAVRh10-hSYN-PGRNwt.

### DETAILED DESCRIPTION OF THE INVENTION

### Definitions

Unless defined otherwise, all technical and scientific terms used herein have the meaning commonly understood by a person skilled in the art to which this invention belongs. The following references provide one of skill with a general definition of many of the terms used in this invention. Singleton et al. Dictionary of Microbiology and Molecular Biology (2^{nd} Ed. 1994); The Cambridge Dictionary of Science and Technology (Walker ed., 1988); The Glossary of Genetics, 5th Ed., R. Rieger et al. (Eds.), Springer Verlag (1991); and Hale & Marham, The Harper Collins Dictionary of Biology (1991). As used herein, the following terms have the meanings ascribed to them below, unless specified otherwise.

The articles “a” and “an” are used herein to refer to one or to more than one (i.e. to at least one) of the grammatical object of the article. By way of example, “an element” means one element or more than one element.

The term “including” is used herein to mean, and is used interchangeably with, the phrase “including but not limited to.”

The term “or” is used herein to mean, and is used interchangeably with, the term “and/or,” unless context clearly indicates otherwise.

The term “such as” is used herein to mean, and is used interchangeably, with the phrase “such as but not limited to.”

As used herein, the terms “administer,” “administering,” “administration,” and the like, are meant to refer to methods that are used to enable delivery of therapeutics or pharmaceutical compositions to the desired site of biological action.

As used herein, the term “AAV virion” is meant to refer broadly to a complete virus particle, such as for example a wild type AAV virion particle, which comprises single stranded genome DNA packaged into AAV capsid proteins. The single stranded nucleic acid molecule is either sense strand or antisense strand, as both strands are equally infectious. The term “rAAV viral particle” refers to a recombinant AAV virus particle, i.e., a particle that is infectious but replication defective. A rAAV viral particle comprises single stranded genome DNA packaged into AAV capsid proteins.

As used herein, the term “bioreactor” is meant to refer broadly to any apparatus that can be used for the purpose of culturing cells.

As used herein, the term “carrier” is meant to include any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutically active substances is well known in the art. Supplementary active ingredients can also be incorporated into the compositions. The phrase “pharmaceutically-acceptable” refers to molecular entities and compositions that do not produce a toxic, an allergic, or similar untoward reaction when administered to a host.

As used herein, the term “flanking” refers to a relative position of one nucleic acid sequence with respect to another nucleic acid sequence. Generally, in the sequence ABC, B is flanked by A and C. The same is true for the arrangement AxBxC. Thus, a flanking sequence precedes or follows a flanked sequence but need not be contiguous with, or immediately adjacent to the flanked sequence.

As used herein, the term “gene delivery” means a process by which foreign DNA is transferred to host cells for applications of gene therapy.

As used herein, the terms “gene” or “coding sequence,” is meant to refer broadly to a DNA region (the transcribed region) which encodes a protein. A coding sequence is transcribed (DNA) and translated (RNA) into a polypeptide when placed under the control of an appropriate regulatory region, such as a promoter. A gene may comprise several operably linked fragments, such as a promoter, a 5′-leader sequence, a coding sequence and a 3′-non-translated sequence, comprising a polyadenylation site. The phrase “expression of a gene” refers to the process wherein a gene is transcribed into an RNA and/or translated into an active protein.

As used herein, the term “gene of interest (GOI),” as used herein refers broadly to a heterologous sequence introduced into an AAV expression vector, and typically refers to a nucleic acid sequence encoding a protein of therapeutic use in humans or animals.

As used herein, the terms “herpesvirus” or “herpesviridae family, are meant to refer broadly to the general family of enveloped, double-stranded DNA viruses with relatively large genomes. The family replicates in the nucleus of a wide range of vertebrate and invertebrate hosts, in preferred embodiments, mammalian hosts, for example in humans, horses, cattle, mice, and pigs. Exemplary members of the herpesviridae family include cytomegalovirus (CMV), herpes simplex virus types 1 and 2 (HSV1 and HSV2) and varicella zoster (VZV) and Epstein Barr Virus (EBV).

As used herein, the term “heterologous,” means derived from a genotypically distinct entity from that of the rest of the entity to which it is compared or into which it is introduced or incorporated. For example, a polynucleotide introduced by genetic engineering techniques into a different cell type is a heterologous polynucleotide (and, when expressed, can encode a heterologous polypeptide). Similarly, a cellular sequence (e.g., a gene or portion thereof) that is incorporated into a viral vector is a heterologous nucleotide sequence with respect to the vector.

As used herein, the term “increase.” “enhance,” “raise” (and like terms) generally refers to the act of increasing, either directly or indirectly, a concentration, level, function, activity, or behavior relative to the natural, expected, or average, or relative to a control condition.

As used herein, the term “infection,” is meant to refer broadly to delivery of heterologous DNA into a cell by a virus. The term “co-infection” as used herein means “simultaneous infection,” “double infection.” “multiple infection,” or “serial infection” with two or more viruses. Infection of a producer cell with two (or more) viruses will be referred to as “co-infection.” The term “transfection” refers to a process of delivering heterologous DNA to a cell by physical or chemical methods, such as plasmid DNA, which is transferred into the cell by means of electroporation, calcium phosphate precipitation, or other methods well known in the art.

As used herein, the term “inverted terminal repeat” or “ITR” sequence is meant to refer to relatively short sequences found at the termini of viral genomes which are in opposite orientation. An “AAV inverted terminal repeat (ITR)” sequence, a term well-understood in the art, is an approximately 145-nucleotide sequence that is present at both termini of the native single-stranded AAV genome. The outermost nucleotides of the ITR can be present in either of two alternative orientations, leading to heterogeneity between different AAV genomes and between the two ends of a single AAV genome.

A “wild-type ITR”, “WT-ITR” or “ITR” refers to the sequence of a naturally occurring ITR sequence in an AAV or other Dependovirus that retains, e.g., Rep binding activity and Rep nicking ability. The nucleotide sequence of a WT-ITR from any AAV serotype may slightly vary from the canonical naturally occurring sequence due to degeneracy of the genetic code or drift, and therefore WT-ITR sequences encompassed for use herein include WT-ITR sequences as result of naturally occurring changes taking place during the production process (e.g., a replication error).

As used herein, the term “terminal repeat” or “TR” includes any viral terminal repeat or synthetic sequence that comprises at least one minimal required origin of replication and a region comprising a palindrome hairpin structure. A Rep-binding sequence (“RBS”) (also referred to as RBE (Rep-binding element)) and a terminal resolution site (“TRS”) together constitute a “minimal required origin of replication” and thus the TR comprises at least one RBS and at least one TRS. TRs that are the inverse complement of one another within a given stretch of polynucleotide sequence are typically each referred to as an “inverted terminal repeat” or “ITR”. In the context of a virus, ITRs mediate replication, virus packaging, integration and provirus rescue.

The term “in vivo” refers to assays or processes that occur in or within an organism, such as a multicellular animal. In some of the aspects described herein, a method or use can be said to occur “in vivo” when a unicellular organism, such as a bacterium, is used. The term “ex vivo” refers to methods and uses that are performed using a living cell with an intact membrane that is outside of the body of a multicellular animal or plant, e.g., explants, cultured cells, including primary cells and cell lines, transformed cell lines, and extracted tissue or cells, including blood cells, among others. The term “in vitro” refers to assays and methods that do not require the presence of a cell with an intact membrane, such as cellular extracts, and can refer to the introducing of a programmable synthetic biological circuit in a non-cellular system, such as a medium not comprising cells or cellular systems, such as cellular extracts.

As used herein, the term “isolated” molecule (e.g., an isolated nucleic acid or protein or cell) means it has been identified and separated and/or recovered from a component of its natural environment.

As used herein, the term “minimal regulatory elements” is meant to refer to regulatory elements that are necessary for effective expression of a gene in a target cell and thus should be included in a transgene expression cassette. Such sequences could include, for example, promoter or enhancer sequences, a polylinker sequence facilitating the insertion of a DNA fragment within a plasmid vector, and sequences responsible for intron splicing and polyadenlyation of mRNA transcripts.

As used herein, the term “minimize”, “reduce”, “decrease,” and/or “inhibit” (and like terms) generally refers to the act of reducing, either directly or indirectly, a concentration, level, function, activity, or behavior relative to the natural, expected, or average, or relative to a control condition.

As used herein, the term “nervous system” includes both the central nervous system and the peripheral nervous system. The term “central nervous system” or “CNS” includes all cells and tissue of the brain and spinal cord of a vertebrate. The term “peripheral nervous system” refers to all cells and tissue of the portion of the nervous system outside the brain and spinal cord. Thus, the term “nervous system” includes, but is not limited to, neuronal cells, glial cells, astrocytes, cells in the cerebrospinal fluid (CSF), cells in the interstitial spaces, cells in the protective coverings of the spinal cord, epidural cells (i.e., cells outside of the dura mater), cells in non-neural tissues adjacent to or in contact with or innervated by neural tissue, cells in the epineurium, perineurium, endoneurium, funiculi, fasciculi, and the like.

As used herein, the term “non-naturally occurring” is meant to refer broadly to a protein, nucleic acid, ribonucleic acid, or virus that does not occur in nature. For example, it may be a genetically modified variant, e.g., cDNA or codon-optimized nucleic acid.

As used herein, a “nucleic acid” or a “nucleic acid molecule” is meant to refer to a molecule composed of chains of monomeric nucleotides, such as, for example, DNA molecules (e.g., cDNA or genomic DNA). A nucleic acid may encode, for example, a promoter, the PGRN gene or portion thereof, or regulatory elements. A nucleic acid molecule can be single-stranded or double-stranded. A “PGRN nucleic acid” refers to a nucleic acid that comprises the PGRN gene or a portion thereof, or a functional variant of the PGRN gene or a portion thereof. A functional variant of a gene includes a variant of the gene with minor variations such as, for example, silent mutations, single nucleotide polymorphisms, missense mutations, and other mutations or deletions that do not significantly alter gene function.

The asymmetric ends of DNA and RNA strands are called the 5′ (five prime) and 3′ (three prime) ends, with the 5′ end having a terminal phosphate group and the 3′ end a terminal hydroxyl group. The five prime (5′) end has the fifth carbon in the sugar-ring of the deoxyribose or ribose at its terminus. Nucleic acids are synthesized in vivo in the 5′- to 3′-direction, because the polymerase used to assemble new strands attaches each new nucleotide to the 3′-hydroxyl (—OH) group via a phosphodiester bond.

The term “nucleic acid construct” as used herein refers to a nucleic acid molecule, either single- or double-stranded, which is isolated from a naturally occurring gene or which is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence of the present disclosure.

A DNA sequence that “encodes” a particular PGRN protein (including fragments and portions thereof) is a nucleic acid sequence that is transcribed into the particular RNA and/or protein. A DNA polynucleotide may encode an RNA (mRNA) that is translated into protein, or a DNA polynucleotide may encode an RNA that is not translated into protein (e.g., tRNA, rRNA, or a DNA-targeting RNA; also called “non-coding” RNA or “ncRNA”).

As used herein, the terms “operatively linked” or “operably linked” or “coupled” can refer to a juxtaposition of genetic elements, wherein the elements are in a relationship permitting them to operate in an expected manner. For instance, a promoter can be operatively linked to a coding region if the promoter helps initiate transcription of the coding sequence. There may be intervening residues between the promoter and coding region so long as this functional relationship is maintained.

As used herein, a “percent (%) sequence identity” with respect to a reference polypeptide or nucleic acid sequence is defined as the percentage of amino acid residues or nucleotides in a candidate sequence that are identical with the amino acid residues or nucleotides in the reference polypeptide or nucleic acid sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various ways that are within the skill in the art, for instance, using publicly available computer software programs, for example, those described in Current Protocols in Molecular Biology (Ausubel et al., eds., 1987), Supp. 30, section 7.7.18, Table 7.7.1, and including BLAST, BLAST-2, ALIGN or Megalign (DNASTAR) software. An example of an alignment program is ALIGN Plus (Scientific and Educational Software, Pennsylvania). Those skilled in the art can determine appropriate parameters for measuring alignment, including any algorithms needed to achieve maximal alignment over the full length of the sequences being compared. For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. For purposes herein, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows: 100 times the fraction W/Z, where W is the number of nucleotides scored as identical matches by the sequence alignment program in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C.

As used herein, the term “pharmaceutical composition” or “composition” is meant to refer to a composition or agent described herein (e.g. a recombinant adeno-associated (rAAV) expression vector), optionally mixed with at least one pharmaceutically acceptable chemical component, such as, though not limited to carriers, stabilizers, diluents, dispersing agents, suspending agents, thickening agents, excipients and the like.

As used herein, the terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

As used herein, the terms “progranulin”, “PGRN”, “granulin-epithelin precursor”, “GEP”, “PC-cell-derived growth factor”, “PCDGF”, “proepithelin”, “acrogranin”, and “GP80” may be used herein interchangeably. As used herein, “a progranulin (PGRN) or “a progranulin (PGRN) nucleic acid” refers to a nucleic acid selected from a nucleic acid comprising SEQ ID NO: 5, a nucleic acid with about 95% homology, about 96%, about 97%, about 98%, about 99% homology with SEQ ID NO: 5, a nucleic acid consisting of SEQ ID NO: 5; a nucleic acid comprising SEQ ID NO: 6, a nucleic acid with about 95% homology, about 96%, about 97%, about 98%, about 99% homology with SEQ ID NO: 6, a nucleic acid consisting of SEQ ID NO: 6; a nucleic acid comprising SEQ ID NO: 7, a nucleic acid with about 95% homology, about 96%, about 97%, about 98%, about 99% homology with SEQ ID NO: 7, a nucleic acid consisting of SEQ ID NO: 7; a nucleic acid comprising SEQ ID NO: 8, a nucleic acid with about 95% homology, about 96%, about 97%, about 98%, about 99% homology with SEQ ID NO: 8, a nucleic acid consisting of SEQ ID NO: 8; a nucleic acid comprising SEQ ID NO: 9, a nucleic acid with about 95% homology, about 96%, about 97%, about 98%, about 99% homology with SEQ ID NO: 9, a nucleic acid consisting of SEQ ID NO: 9; a nucleic acid comprising SEQ ID NO: 10, a nucleic acid with about 95% homology, about 96%, about 97%, about 98%, about 99% homology with SEQ ID NO: 10, a nucleic acid consisting of SEQ ID NO: 10; a nucleic acid comprising SEQ ID NO: 11, a nucleic acid with about 95% homology, about 96%, about 97%, about 98%, about 99% homology with SEQ ID NO: 11, a nucleic acid consisting of SEQ ID NO: 11; a nucleic acid comprising SEQ ID NO: 12, a nucleic acid with about 95% homology, about 96%, about 97%, about 98%, about 99% homology with SEQ ID NO: 12, a nucleic acid consisting of SEQ ID NO: 12; a nucleic acid comprising SEQ ID NO: 13, a nucleic acid with about 95% homology, about 96%, about 97%, about 98%, about 99% homology with SEQ ID NO: 13, a nucleic acid consisting of SEQ ID NO: 13; or a nucleic acid comprising SEQ ID NO: 14, a nucleic acid with about 95% homology, about 96%, about 97%, about 98%, about 99% homology with SEQ ID NO: 14, a nucleic acid consisting of SEQ ID NO: 14

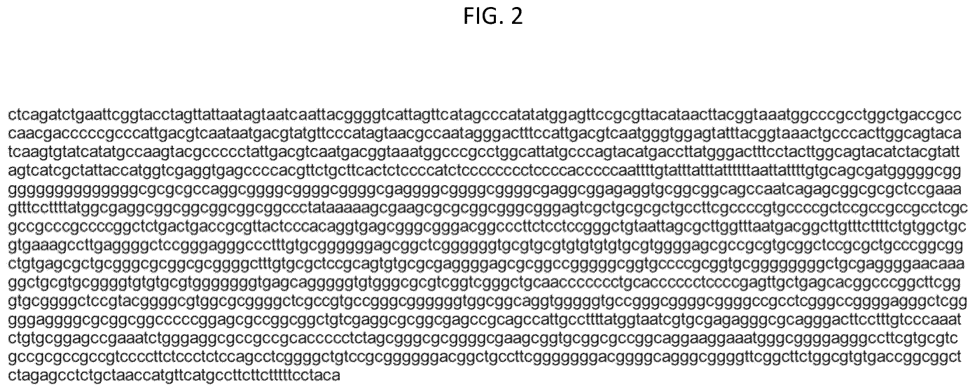

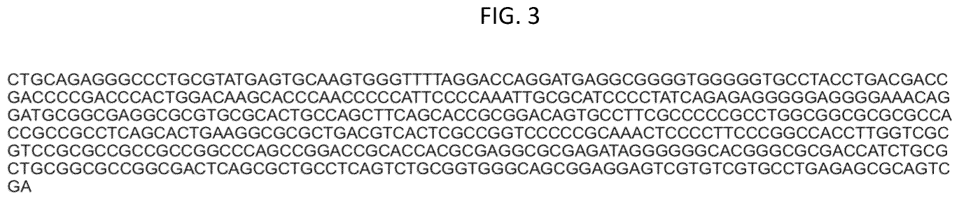

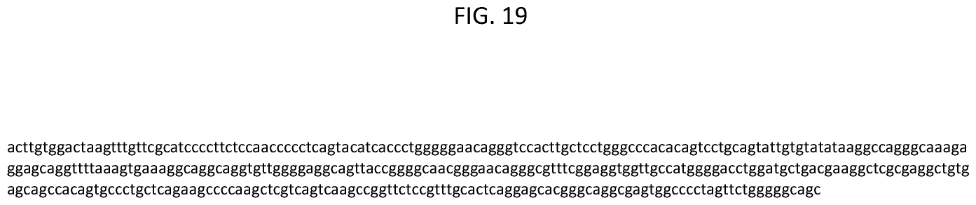

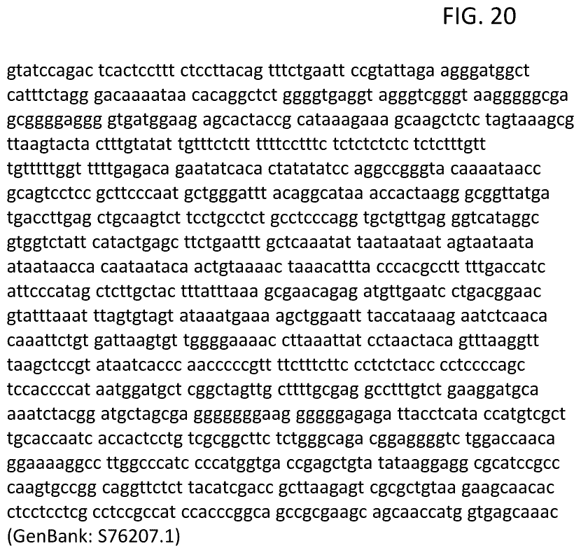

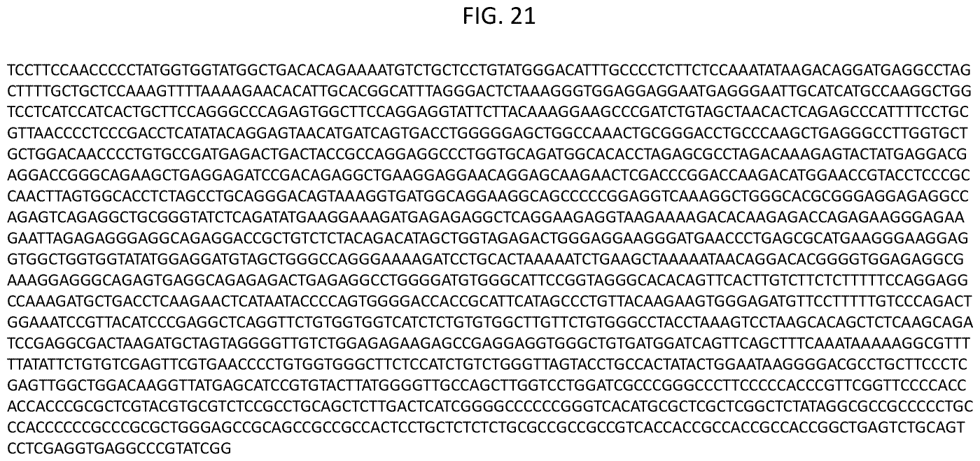

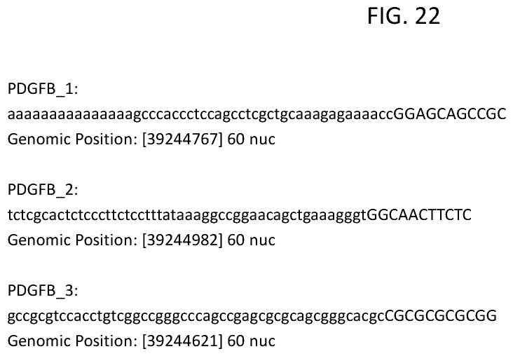

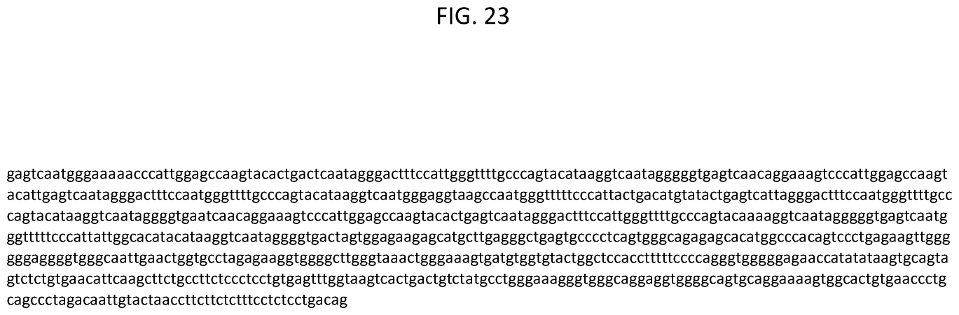

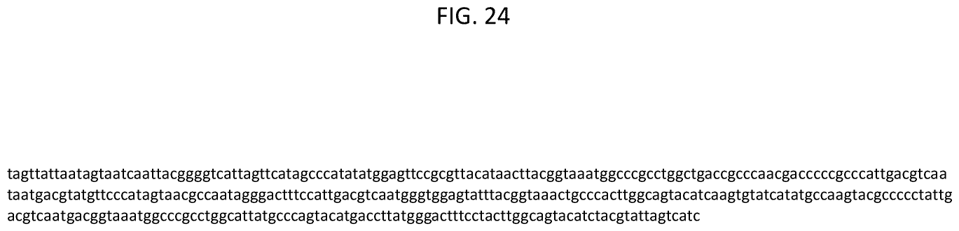

As used herein, a “promoter” is meant to refer to a nucleotide region comprising a DNA regulatory sequence, wherein the regulatory sequence is derived from a gene which is capable of binding RNA polymerase and initiating transcription of a downstream (3′-direction) coding sequence. As part of the process of transcription, the enzyme that synthesizes RNA, known as RNA polymerase, attaches to the DNA near a gene. Promoters contain specific DNA sequences and response elements that provide an initial binding site for RNA polymerase and for transcription factors that recruit RNA polymerase. According to some embodiments, the promoter is highly specific for transgene expression in cells of the CNS. According to some embodiments, the promoter is highly specific for neuron-specific transgene expression. According to some embodiments, the promoter is an endogenous PGRN promoter. According to some embodiments, the promoter is a chicken beta-actin (CBA) promoter. According to some embodiments, the promoter is a human synapsin-1 gene promoter (hSyn1) promoter. FIG. 3 shows the nucleic acid sequence of the human synapsin 1 (hSYN1) promoter (SEQ ID NO: 2). According to some embodiments, the hSYN1 promoter comprises SEQ ID NO: 2. According to some embodiments, the hSYN1 promoter consists of SEQ ID NO: 2. A CBA promoter refers to a polynucleotide sequence derived from a chicken beta-actin gene (e.g., Gallus gallus beta actin, represented by GenBank Entrez Gene ID 396526). FIG. 2 shows the nucleic acid sequence of the chicken beta actin (CBA) promoter (SEQ ID NO: 1). According to some embodiments, the CBA promoter comprises SEQ ID NO: 1. According to some embodiments, the CBA promoter consists of SEQ ID NO: 1. According to some embodiments, the promoter is a mouse calcium/calmodulin-dependent protein kinase II (CaMKII) promoter. The mouse calcium/calmodulin-dependent protein kinase II promoter is a moderate strength neuron-specific promoter. FIG. 19 shows the nucleic acid sequence of CaMKII promoter (SEQ ID NO: 18). According to some embodiments, the CaMKII promoter comprises SEQ ID NO: 18. According to some embodiments, the CaMKII promoter consists of SEQ ID NO: 18. According to some embodiments, the promoter is a rat tubulin alpha 1 (Ta1) promoter. The rat tubulin alpha 1 promoter is a moderate strength promoter responsible for regulating neuron gene expression as a function of morphological growth. The rat Ta1 promoter is described in Gloster et al. 1994 J of Neuroscience, incorporated by reference in its entirety herein. FIG. 20 shows the nucleic acid sequence of Rat tubulin alpha 1 (Ta1) promoter (SEQ ID NO: 19). According to some embodiments, the Ta1 promoter comprises SEQ ID NO: 19. According to some embodiments, the Ta1 promoter consists of SEQ ID NO: 19. According to some embodiments, the promoter is a rat neuron-specific enolase (NSE) promoter. The rat neuron-specific enolase promoter is a moderate strength, developmentally regulated promoter. FIG. 21 shows the nucleic acid sequence of Rat neuron-specific enolase (NSE) promoter (SEQ ID NO: 20). According to some embodiments, the NSE promoter comprises SEQ ID NO: 20. According to some embodiments, the NSE promoter consists of SEQ ID NO: 20. According to some embodiments, the promoter is a human platelet-derived growth factor-beta chain (PDGF) promoter. The human platelet-derived growth factor-beta chain promoter is a moderate strength promoter specific to neurons and (neuron-associated) glial cells. There are three defined promoters in human genome, spanning location 39244982-39244621 on the [-] strand of chromosome NC_000022.11 (spans 362 nucleotides). FIG. 22 shows the nucleic acid sequence of a human platelet-derived growth factor-beta chain (PDGF) promoter (SEQ ID NO: 21, SEQ ID NO: 24 and SEQ ID NO: 25). According to some embodiments, the PDGF-beta chain promoter comprises SEQ ID NO: 21. According to some embodiments, the PDGF-beta chain promoter consists of SEQ ID NO: 21. According to some embodiments, the promoter is an ubiquitously-active EF1alpha promoter. The EF1a promoter is a strong ubiquitous promoter of mammalian origin that expresses in most cell types, including cells of the CNS. FIG. 23 shows the nucleic acid sequence of EF1alpha promoter (SEQ ID NO: 22). According to some embodiments, the EF1alpha promoter comprises SEQ ID NO: 22. According to some embodiments, the EF1alpha promoter consists of SEQ ID NO: 22. According to some embodiments, any of the promoters set forth in the aspects and embodiments herein further comprise an additional 5′ CAG/CMV enhancer element. The CAG/CMV enhancer is a strong enhancer element derived from the enhancer that affects the strong ubiquitous CAG promoter and it's CMV parent promoter; both are commonly employed to enhance transcriptional activity of core promoters. FIG. 24 shows the nucleic acid sequence of a CAG/CMV enhancer (SEQ ID NO: 23).

A promoter can be said to drive expression or drive transcription of the nucleic acid sequence that it regulates. The phrases “operably linked.” “operatively positioned.” “operatively linked,” “under control,” and “under transcriptional control” indicate that a promoter is in a correct functional location and/or orientation in relation to a nucleic acid sequence it regulates to control transcriptional initiation and/or expression of that sequence. An “inverted promoter.” as used herein, refers to a promoter in which the nucleic acid sequence is in the reverse orientation, such that what was the coding strand is now the non-coding strand, and vice versa. Inverted promoter sequences can be used in various embodiments to regulate the state of a switch. In addition, in various embodiments, a promoter can be used in conjunction with an enhancer.

A promoter can be one naturally associated with a gene or sequence, as can be obtained by isolating the 5′ non-coding sequences located upstream of the coding segment and/or exon of a given gene or sequence. Such a promoter can be referred to as “endogenous.” Similarly, in some embodiments, an enhancer can be one naturally associated with a nucleic acid sequence, located either downstream or upstream of that sequence.

In some embodiments, a coding nucleic acid segment is positioned under the control of a “recombinant promoter” or “heterologous promoter.” both of which refer to a promoter that is not normally associated with the encoded nucleic acid sequence it is operably linked to in its natural environment. A recombinant or heterologous enhancer refers to an enhancer not normally associated with a given nucleic acid sequence in its natural environment. Such promoters or enhancers can include promoters or enhancers of other genes; promoters or enhancers isolated from any other prokaryotic, viral, or eukaryotic cell; and synthetic promoters or enhancers that are not “naturally occurring.” i.e., comprise different elements of different transcriptional regulatory regions, and/or mutations that alter expression through methods of genetic engineering that are known in the art.

The term “enhancer” as used herein refers to a cis-acting regulatory sequence (e.g., 50-1,500 base pairs) that binds one or more proteins (e.g., activator proteins, or transcription factor) to increase transcriptional activation of a nucleic acid sequence. Enhancers can be positioned up to 1,000,000 base pars upstream of the gene start site or downstream of the gene start site that they regulate.

As used herein, the term “recombinant” can refer to a biomolecule, e.g., a gene or protein, that (1) has been removed from its naturally occurring environment. (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids.

As used herein, the term “recombinant HSV.” “rHSV,” and “rHSV vector,” is meant to refer broadly to isolated, genetically modified forms of herpes simplex virus type 1 (HSV) containing heterologous genes incorporated into the viral genome. By the term “rHSV-rep2cap2” or “rHSV-rep2cap1” is meant an rHSV in which the AAV rep and cap genes from either AAV serotype 1 or 2 have been incorporated into the rHSV genome, in certain embodiments, a DNA sequence encoding a therapeutic gene of interest has been incorporated into the viral genome.

As used herein, a “subject” or “patient” or “individual” to be treated by the method of the invention is meant to refer to either a human or non-human animal. A “nonhuman animal” includes any vertebrate or invertebrate organism. A human subject can be of any age, gender, race or ethnic group, e.g., Caucasian (white), Asian, African, black, African American, African European, Hispanic, Middle eastern, etc. In some embodiments, the subject can be a patient or other subject in a clinical setting. In some embodiments, the subject is already undergoing treatment. In some embodiments, the subject is a neonate, infant, child, adolescent, or adult.

As used herein the term “therapeutic effect” refers to a consequence of treatment, the results of which are judged to be desirable and beneficial. A therapeutic effect can include, directly or indirectly, the arrest, reduction, or elimination of a disease manifestation. A therapeutic effect can also include, directly or indirectly, the arrest reduction or elimination of the progression of a disease manifestation.

For any therapeutic agent described herein therapeutically effective amount may be initially determined from preliminary in vitro studies and/or animal models. A therapeutically effective dose may also be determined from human data. The applied dose may be adjusted based on the relative bioavailability and potency of the administered compound. Adjusting the dose to achieve maximal efficacy based on the methods described above and other well-known methods is within the capabilities of the ordinarily skilled artisan. General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference, are summarized below.

As used herein, the term “substitution mutation profile” is meant to refer to a panel of conservative amino acid substitutions in the inter-domain regions of progranulin that eliminate one or more of its 5 elastase cleavage sites and/or 2 other proteolytic cleavage sites (see e.g., Cenik et al., JBC 2012; Zhu et al., Cell 2002, both of which are incorporated by reference in their entireties herein). According to some embodiments, the substitution mutations will reduce or prevent PGRN from being processed into granulins.

As used herein, the term “transgene” is meant to refer to a polynucleotide that is introduced into a cell and is capable of being transcribed into RNA and optionally, translated and/or expressed under appropriate conditions. In aspects, it confers a desired property to a cell into which it was introduced, or otherwise leads to a desired therapeutic or diagnostic outcome.

As used herein, a “transgene expression cassette” or “expression cassette” are used interchangeably and refer to a linear stretch of nucleic acids that includes a transgene that is operably linked to one or more promoters or other regulatory sequences sufficient to direct transcription of the transgene, but which does not comprise capsid-encoding sequences, other vector sequences or inverted terminal repeat regions. An expression cassette may additionally comprise one or more cis-acting sequences (e.g., promoters, enhancers, or repressors), one or more introns, and one or more post-transcriptional regulatory elements. A transgene expression cassette comprises the gene sequences that a nucleic acid vector is to deliver to target cells. These sequences include the gene of interest (e.g., PGRN nucleic acids or variants thereof), one or more promoters, and minimal regulatory elements.

As used herein, the terms “treatment” or “treating” a disease or disorder are meant to refer to alleviation of one or more signs or symptoms of the disease or disorder, diminishment of extent of disease or disorder, stabilized (e.g., not worsening) state of disease or disorder, preventing spread of disease or disorder, delay or slowing of disease or disorder progression, amelioration or palliation of the disease or disorder state, and remission (whether partial or total), whether detectable or undetectable. For example, PGRN, when expressed in an effective amount (or dosage) is sufficient to prevent, correct, and/or normalize an abnormal physiological response, e.g., a therapeutic effect that is sufficient to reduce by at least about 30 percent, more preferably by at least 50 percent, most preferably by at least 90 percent, a clinically significant feature of disease or disorder. “Treatment” can also refer to prolonging survival as compared to expected survival if not receiving treatment.

As used herein, the term “vector” is meant to refer to a recombinant plasmid or virus that comprises a nucleic acid to be delivered into a host cell, either In vitro or in vivo.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g., 5′ untranslated (5′UTR) or “leader” sequences and 3′ UTR or “trailer” sequences, as well as intervening sequences (introns) between individual coding segments (exons).

As used herein, the term “recombinant viral vector” is meant to refer to a recombinant polynucleotide vector comprising one or more heterologous sequences (i.e., nucleic acid sequence not of viral origin). In the case of recombinant AAV vectors, the recombinant nucleic acid is flanked by at least one inverted terminal repeat sequence (ITR). According to some embodiments, the recombinant nucleic acid is flanked by two ITRs.

As used herein, the term “recombinant AAV vector (rAAV vector)” is meant to refer to a polynucleotide vector comprising one or more heterologous sequences (i.e., nucleic acid sequence not of AAV origin) that are flanked by at least one AAV inverted terminal repeat sequence (ITR). Such rAAV vectors can be replicated and packaged into infectious viral particles when present in a host cell that has been infected with a suitable helper virus (or that is expressing suitable helper functions) and that is expressing AAV rep and cap gene products (i.e. AAV Rep and Cap proteins). When a rAAV vector is incorporated into a larger polynucleotide (e.g., in a chromosome or in another vector such as a plasmid used for cloning or transfection), then the rAAV vector may be referred to as a “pro-vector” which can be “rescued” by replication and encapsidation in the presence of AAV packaging functions and suitable helper functions. A rAAV vector can be in any of a number of forms, including, but not limited to, plasmids, linear artificial chromosomes, complexed with lipids, encapsulated within liposomes, and encapsidated in a viral particle, e.g., an AAV particle. A rAAV vector can be packaged into an AAV virus capsid to generate a “recombinant adeno-associated viral particle (rAAV particle)”.

As used herein, the term a “rAAV virus” or “rAAV viral particle” is meant to refer to a viral particle composed of at least one AAV capsid protein and an encapsidated rAAV vector genome.

As used herein, “reporters” refer to proteins that can be used to provide detectable read-outs. Reporters generally produce a measurable signal such as fluorescence, color, or luminescence. Reporter protein coding sequences encode proteins whose presence in the cell or organism is readily observed. For example, fluorescent proteins cause a cell to fluoresce when excited with light of a particular wavelength, luciferases cause a cell to catalyze a reaction that produces light, and enzymes such as β-galactosidase convert a substrate to a colored product. Exemplary reporter polypeptides useful for experimental or diagnostic purposes include, but are not limited to β-lactamase, β-galactosidase (LacZ), alkaline phosphatase (AP), thymidine kinase (TK), green fluorescent protein (GFP) and other fluorescent proteins, chloramphenicol acetyltransferase (CAT), luciferase, and others well known in the art.

Transcriptional regulators refer to transcriptional activators and repressors that either activate or repress transcription of a gene of interest, such as PGRN. Promoters are regions of nucleic acid that initiate transcription of a particular gene Transcriptional activators typically bind nearby to transcriptional promoters and recruit RNA polymerase to directly initiate transcription. Repressors bind to transcriptional promoters and sterically hinder transcriptional initiation by RNA polymerase. Other transcriptional regulators may serve as either an activator or a repressor depending on where they bind and cellular and environmental conditions. Non-limiting examples of transcriptional regulator classes include, but are not limited to homeodomain proteins, zinc-finger proteins, winged-helix (forkhead) proteins, and leucine-zipper proteins.

As used herein, a “repressor protein” or “inducer protein” is a protein that binds to a regulatory sequence element and represses or activates, respectively, the transcription of sequences operatively linked to the regulatory sequence element. Preferred repressor and inducer proteins as described herein are sensitive to the presence or absence of at least one input agent or environmental input. Preferred proteins as described herein are modular in form, comprising, for example, separable DNA-binding and input agent-binding or responsive elements or domains.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The term “including” is used herein to mean, and is used interchangeably with, the phrase “including but not limited to.”

The term “such as” is used herein to mean, and is used interchangeably, with the phrase “such as but not limited to.”

As used in this specification and the appended claims, the singular forms “a,” “an,” and “the” include plural references unless the context clearly dictates otherwise. Thus, for example, references to “the method” includes one or more methods, and/or steps of the type described herein and/or which will become apparent to those persons skilled in the art upon reading this disclosure and so forth. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

### II. Nucleic Acids

The characterization and development of nucleic acid molecules for potential therapeutic use are provided herein. The present disclosure provides promoters, expression cassettes, vectors, kits, and methods that can be used in the treatment of neurodegenerative diseases or disorders. Certain aspects of the disclosure relate to delivering a heterologous nucleic acid to a subject comprising administering a recombinant adeno-associated virus (rAAV) vector. According to some aspects, the disclosure provides methods of treating or preventing neurodegenerative diseases or disorders comprising delivery of a composition comprising rAAV vectors described herein to the subject, wherein the rAAV vector comprises a heterologous nucleic acid (e.g. a nucleic acid encoding PGRN). An object of the present disclosure is to deliver nucleic acids encoding PGRN to the central nervous system (CNS) with successful expression in the CNS, and the treatment of neurodegenerative disease.

According to some embodiments, the expressed PGRN protein is functional for the treatment of treatment of a neurodegenerative disease or disorders. In some embodiments, expressed PGRN protein does not cause an immune system reaction.

PGRN is a glycoprotein encoded by the GRN/Grn gene with multiple cellular functions, including neurotrophic, anti-inflammatory and lysosome regulatory properties. Mutations in the GRN gene can lead to frontotemporal lobar degeneration (FTD), a cause of dementia, and neuronal ceroid lipofuscinosis (NCL), a lysosomal storage disease. Both diseases are associated with loss of PGRN function resulting, amongst other features, in enhanced microglial neuroinflammation and lysosomal dysfunction. PGRN has also been implicated in Alzheimer's disease (AD). Mendsaikhan et al. Cells. 2019 8 (3): 230.

According to some embodiments, the gene of interest (e.g., PGRN) is optimized to be superior in expression (and/or function) to wildtype PGRN, and further has the ability to discriminate (at the DNA/RNA level) from wildtype PGRN.

According to some embodiments, the gene of interest (e.g., PGRN) is optimized to inhibit binding to sortilin. It has been shown previously that the PGRN C-terminal motif, PGRN (589-593) LRQLL, is essential for SORT1-mediated endocytosis (Zheng et al., PLOS One. 2011; 6 (6): e21023).

According to some embodiments, the gene of interest (e.g., PGRN) is optimized to generate less granulin products.

A “PGRN nucleic acid” refers to a nucleic acid that comprises the PGRN gene or a portion thereof, or a functional variant of the PGRN gene or a portion thereof. A functional variant of a gene includes a variant of the gene with minor variations such as, for example, silent mutations, single nucleotide polymorphisms, missense mutations, and other mutations or deletions that do not significantly alter gene function.

According to one embodiment, the nucleic acid encoding the PGRN protein is 1779 bp in length. According to one embodiment, the nucleic acid comprises SEQ ID NO: 5. According to one embodiment, the nucleic acid is at least 85% identical to SEQ ID NO: 5. According to one embodiment, the nucleic acid is at least 90% identical to SEQ ID NO: 5. According to one embodiment, the nucleic acid is at least 95% identical to SEQ ID NO: 5. According to one embodiment, the nucleic acid is at least 96% identical to SEQ ID NO: 5. According to one embodiment, the nucleic acid is at least 96% identical to SEQ ID NO: 5. According to one embodiment, the nucleic acid is at least 97% identical to SEQ ID NO: 5. According to one embodiment, the nucleic acid is at least 98% identical to SEQ ID NO: 5. According to one embodiment, the nucleic acid is at least 99% identical to SEQ ID NO: 5. According to one embodiment, the nucleic acid consists of SEQ ID NO: 5.

According to one embodiment, the nucleic acid encoding the PGRN protein is 1767 bp in length. According to one embodiment, the nucleic acid comprises SEQ ID NO: 6. According to one embodiment, the nucleic acid is at least 85% identical to SEQ ID NO: 6. According to one embodiment, the nucleic acid is at least 90% identical to SEQ ID NO: 6. According to one embodiment, the nucleic acid is at least 95% identical to SEQ ID NO: 6. According to one embodiment, the nucleic acid is at least 96% identical to SEQ ID NO: 6. According to one embodiment, the nucleic acid is at least 97% identical to SEQ ID NO: 6. According to one embodiment, the nucleic acid is at least 98% identical to SEQ ID NO: 6. According to one embodiment, the nucleic acid is at least 99% identical to SEQ ID NO: 6. According to one embodiment, the nucleic acid consists of SEQ ID NO: 6.

According to one embodiment, the nucleic acid encoding the PGRN protein is 1779 bp in length. According to one embodiment, the nucleic acid comprises SEQ ID NO: 7. According to one embodiment, the nucleic acid is at least 85% identical to SEQ ID NO: 7. According to one embodiment, the nucleic acid is at least 90% identical to SEQ ID NO: 7. According to one embodiment, the nucleic acid is at least 95% identical to SEQ ID NO: 7. According to one embodiment, the nucleic acid is at least 96% identical to SEQ ID NO: 7. According to one embodiment, the nucleic acid is at least 97% identical to SEQ ID NO: 7. According to one embodiment, the nucleic acid is at least 98% identical to SEQ ID NO: 7. According to one embodiment, the nucleic acid is at least 99% identical to SEQ ID NO: 7. According to one embodiment, the nucleic acid consists of SEQ ID NO: 7.

According to one embodiment, the nucleic acid encoding the PGRN protein is 1779 bp in length. According to one embodiment, the nucleic acid comprises SEQ ID NO: 8. According to one embodiment, the nucleic acid is at least 85% identical to SEQ ID NO: 8. According to one embodiment, the nucleic acid is at least 90% identical to SEQ ID NO: 8. According to one embodiment, the nucleic acid is at least 95% identical to SEQ ID NO: 8. According to one embodiment, the nucleic acid is at least 96% identical to SEQ ID NO: 8. According to one embodiment, the nucleic acid is at least 97% identical to SEQ ID NO: 8. According to one embodiment, the nucleic acid is at least 98% identical to SEQ ID NO: 8. According to one embodiment, the nucleic acid is at least 99% identical to SEQ ID NO: 8. According to one embodiment, the nucleic acid consists of SEQ ID NO: 8.

According to one embodiment, the nucleic acid encoding the PGRN protein is 1779 bp in length. According to one embodiment, the nucleic acid comprises SEQ ID NO: 9. According to one embodiment, the nucleic acid is at least 85% identical to SEQ ID NO: 9. According to one embodiment, the nucleic acid is at least 90% identical to SEQ ID NO: 9. According to one embodiment, the nucleic acid is at least 95% identical to SEQ ID NO: 9. According to one embodiment, the nucleic acid is at least 96% identical to SEQ ID NO: 9. According to one embodiment, the nucleic acid is at least 97% identical to SEQ ID NO: 9. According to one embodiment, the nucleic acid is at least 98% identical to SEQ ID NO: 9. According to one embodiment, the nucleic acid is at least 99% identical to SEQ ID NO: 9. According to one embodiment, the nucleic acid consists of SEQ ID NO: 9.

According to one embodiment, the nucleic acid encoding the PGRN protein is 1779 bp in length. According to one embodiment, the nucleic acid comprises SEQ ID NO: 10. According to one embodiment, the nucleic acid is at least 85% identical to SEQ ID NO: 10. According to one embodiment, the nucleic acid is at least 90% identical to SEQ ID NO: 10. According to one embodiment, the nucleic acid is at least 95% identical to SEQ ID NO: 10. According to one embodiment, the nucleic acid is at least 96% identical to SEQ ID NO: 10. According to one embodiment, the nucleic acid is at least 97% identical to SEQ ID NO: 10. According to one embodiment, the nucleic acid is at least 98% identical to SEQ ID NO: 10. According to one embodiment, the nucleic acid is at least 99% identical to SEQ ID NO: 10. According to one embodiment, the nucleic acid consists of SEQ ID NO: 10.

According to one embodiment, the nucleic acid encoding the PGRN protein is 1779 bp in length. According to one embodiment, the nucleic acid comprises SEQ ID NO: 11. According to one embodiment, the nucleic acid is at least 85% identical to SEQ ID NO: 11. According to one embodiment, the nucleic acid is at least 90% identical to SEQ ID NO: 11. According to one embodiment, the nucleic acid is at least 95% identical to SEQ ID NO: 11. According to one embodiment, the nucleic acid is at least 96% identical to SEQ ID NO: 11. According to one embodiment, the nucleic acid is at least 97% identical to SEQ ID NO: 11. According to one embodiment, the nucleic acid is at least 98% identical to SEQ ID NO: 11. According to one embodiment, the nucleic acid is at least 99% identical to SEQ ID NO: 8. According to one embodiment, the nucleic acid consists of SEQ ID NO: 11.

According to one embodiment, the nucleic acid encoding the PGRN protein is 1779 bp in length. According to one embodiment, the nucleic acid comprises SEQ ID NO: 12. According to one embodiment, the nucleic acid is at least 85% identical to SEQ ID NO: 12. According to one embodiment, the nucleic acid is at least 90% identical to SEQ ID NO: 12. According to one embodiment, the nucleic acid is at least 95% identical to SEQ ID NO: 12. According to one embodiment, the nucleic acid is at least 96% identical to SEQ ID NO: 12. According to one embodiment, the nucleic acid is at least 97% identical to SEQ ID NO: 12. According to one embodiment, the nucleic acid is at least 98% identical to SEQ ID NO: 12. According to one embodiment, the nucleic acid is at least 99% identical to SEQ ID NO: 12. According to one embodiment, the nucleic acid consists of SEQ ID NO: 12.

According to one embodiment, the nucleic acid encoding the PGRN protein is 1779 bp in length. According to one embodiment, the nucleic acid comprises SEQ ID NO: 13. According to one embodiment, the nucleic acid is at least 85% identical to SEQ ID NO: 13. According to one embodiment, the nucleic acid is at least 90% identical to SEQ ID NO: 13. According to one embodiment, the nucleic acid is at least 95% identical to SEQ ID NO: 13. According to one embodiment, the nucleic acid is at least 96% identical to SEQ ID NO: 13. According to one embodiment, the nucleic acid is at least 97% identical to SEQ ID NO: 13. According to one embodiment, the nucleic acid is at least 98% identical to SEQ ID NO: 13. According to one embodiment, the nucleic acid is at least 99% identical to SEQ ID NO: 13. According to one embodiment, the nucleic acid consists of SEQ ID NO: 13.

According to one embodiment, the nucleic acid encoding the PGRN protein has a deletion of 3-16 (e.g., 3, 4, 5, 6, 7, 8, 9, 10, 11, 12, 13, 14, 15, 16) amino acids from the C-terminus. According to some embodiments, the deletion in the C-terminus of PGRN results in inhibition of PGRN sortilin binding and subsequent processing to individual granulins. According to some embodiments, the nucleic acid encoding the PGRN protein consists of SEQ ID NO: 1 with a deletion of 3-16 (e.g., 3, 4, 5, 6, 7, 8, 9, 10, 11, 12, 13, 14, 15, 16) amino acids from the C-terminus.

### Codon-Optimization

A polynucleotide encoding a site-directed polypeptide can be codon-optimized according to methods standard in the art for expression in the cell containing the target DNA of interest. For example, if the intended target nucleic acid is in a human cell, a human codon-optimized polynucleotide encoding PGRN is contemplated for use in the constructs described herein.

According to some embodiments, the nucleic acid sequence is codon optimized for mammalian expression.

### III. PGRN Gene Therapy for Neurodegenerative Diseases

The disclosure generally provides methods for producing recombinant adeno-associated virus (AAV) viral particles comprising a PGRN gene construct and their use in methods of gene therapy for neurodegenerative diseases, and in particular neurodegenerative diseases characterized by partial or complete PGRN deficiency, e.g., frontotemporal dementia (FTD). The AAV vectors as described herein are particularly efficient at delivering nucleic acids (e.g., PGRN gene construct) to cells of the CNS, and in particular to neuronal cells. Methods to create, evaluate, and utilize recombinant adeno-associated virus (rAAV) therapeutic vectors capable of efficiently delivering PGRN into cells for expression and subsequent secretion are described herein. Optimally-modified PGRN cDNA and associated genetic elements for use in recombinant adeno-associated virus (rAAV)-based gene therapy for neurodegenerative diseases, including the treatment and/or prevention of FTD, are described herein.

Recombinant adeno-associated virus (rAAV) vector can efficiently accommodate both PGRN target gene and associated genetic elements. Furthermore, such vectors can be designed to specifically express PGRN in therapeutically relevant cells of the CNS. The disclosure describes a method to create, evaluate, and utilize rAAV therapeutic vectors able to efficiently deliver the functional PGRN gene to patients.

The PGRN gene construct may comprise: (1) a 1.8 kilobase (kb) non-naturally occurring codon-optimized PGRN cDNA sequence, derived from either human, mouse, or macaque, which may possess resistance to proteolytic cleavage to granulins by means of either (a) a substitution mutation profile or by a 3-16 C-terminal amino acid deletion (to inhibit sortilin binding and subsequent processing to individual granulins), with or without a 27-nucleotide hemagglutinin C-terminal tag. (2) a 0.5 kb non-naturally occurring neuron-specific human synapsin-1 promoter (hSYN1) or 0.364 kb mouse calcium/calmodulin-dependent protein kinase II (CaMKII) promoter, or 1.034 kb rat tubulin alpha 1 (Ta1) promoter, or 1.81 kb rat neuron-specific enolase (NSE) promoter, or 1.47 kb human platelet-derived growth factor-beta chain (PDGF) promoter, or ubiquitously-active 1.7 kb CBA promoter, or ubiquitously-active 0.81 kb EF1alpha promoter, or any of the promoters according to any of the aspects or embodiments herein, wherein the promoter further comprises an additional 0.35 kb 5′ CAG/CMV enhancer element, all optimized to drive high PGRN expression. (3) a 0.9 kb non-naturally occurring 3′-UTR regulatory region comprising the Woodchuck Hepatitis Virus Posttranscriptional Regulatory Element (WPRE) followed by either a SV40 or human growth hormone (hGH) polyadenylation signal, (4) two naturally occurring 141-base sequence-modulated inverted terminal repeats (ITRs) flanking the AAV genomic cassette, and (5) a AAV capsid variant (either naturally or non-occurring) optimally suited for targeted CNS delivery.

### Self-Complementary AAV Genome

Numerous preclinical studies have demonstrated the efficacy of recombinant adeno-associated virus (rAAV) gene delivery vectors, and recent clinical trials have shown promising results. However, the efficiency of these vectors, in terms of the number of genome-containing particles required for transduction, is hindered by the need to convert the single-stranded DNA (ssDNA) genome into double-stranded DNA (dsDNA) prior to expression. This step can be entirely circumvented through the use of self-complementary vectors, which package an inverted repeat genome that can fold into dsDNA without the requirement for DNA synthesis or base-pairing between multiple vector genomes. The important trade-off for this efficiency is the loss of half the coding capacity of the vector, though small protein-coding genes (up to 55 kd), and any currently available RNA-based therapy, can be accommodated.

### Adeno-Associated Virus (AAV)

Adeno-Associated Virus (AAV) is a non-pathogenic single-stranded DNA parvovirus. AAV has a capsid diameter of about 20 nm. Each end of the single-stranded DNA genome contains an inverted terminal repeat (ITR), which is the only cis-acting element required for genome replication and packaging. The AAV genome carries two viral genes: rep and cap. The virus utilizes two promoters and alternative splicing to generate four proteins necessary for replication (Rep 78, Rep 68, Rep 52 and Rep 40). A third promoter generates the transcript for three structural viral capsid proteins, 1, 2 and 3 (VP1, VP2 and VP3), through a combination of alternate splicing and alternate translation start codons. Berns & Linden Bioessays 1995; 17:237-45. The three capsid proteins share the same C-terminal 533 amino acids, while VP2 and VP1 contain additional N-terminal sequences of 65 and 202 amino acids, respectively. The AAV virion contains a total of 60 copies of VP1, VP2, and VP3 at a 1:1:20 ratio, arranged in a T-1 icosahedral symmetry. Rose et al. J Virol. 1971; 8:766-70. AAV requires Adenovirus (Ad), Herpes Simplex Virus (HSV) or other viruses as a helper virus to complete its lytic life-cycle. Atchison et al. Science, 1965; 149:754-6; Hoggan et al. Proc Natl Acad Sci USA, 1966; 55:1467-74. In the absence of the helper virus, wild-type AAV establishes latency by integration with the assistance of Rep proteins through the interaction of the ITR with the chromosome. Berns & Linden (1995).

### AAV Serotypes

There are a number of different AAV serotypes, including AAV1, AAV2, AAV3, AAV4, AAV5, AAV6, AAV7, AAV8, AAV9, AAV10, AAV11, AAV12, AAVrh8, AAVrh10, AAV2.retro, and variants or hybrids thereof (e.g. AAV2 variants with HSPG mutation, AAV 1+9 hybrids). In vivo studies have shown that the various AAV serotypes display different tissue or cell tropisms. For example, AAV1 and AAV6 are two serotypes that, are efficient for the transduction of skeletal muscle. Gao, et al. Proc Natl Acad Sci USA, 2002; 99:11854-11859; Xiao, et al. J Virol. 1999; 73:3994-4003; Chao, et al. Mol Ther. 2000; 2:619-623. AAV-3 has been shown to be superior for the transduction of megakaryocytes. Handa, et al. J Gen Virol. 2000; 81:2077-2084. AAV5 and AAV6 infect apical airway cells efficiently. Zabner, et al. J Virol. 2000; 74:3852-3858; Halbert, et al. J Virol. 2001; 75:6615-6624. AAV2, AAV4, and AAV5 transduce different types of cells in the central nervous system. Davidson, et al. Proc Natl Acad Sci USA. 2000; 97:3428-3432. AAV8 and AAV5 can transduce liver cells better than AAV-2. AAV-5 based vectors transduced certain cell types (cultured airway epithelial cells, cultured striated muscle cells and cultured human umbilical vein endothelial cells) at a higher efficiency than AAV2, while both AAV2 and AAV5 showed poor transduction efficiencies for NIH 3T3, skbr3 and t-47D cell lines. Gao, et al. Proc Natl Acad Sci USA. 2002; 99:11854-11859; Mingozzi, et al. J Virol. 2002; 76:10497-10502. WO 99/61601. AAV4 was found to transduce rat retina most efficiently, followed by AAV5 and AAV1. Rabinowitz, et al. J Virol. 2002; 76:791-801; Weber, et al. Mol Ther. 2003; 7:774-781. In summary, AAV1, AAV2, AAV4, AAV5, AAV8, and AAV9 show tropism for CNS tissues. AAV1, AAV8, and AAV9 show tropism for heart tissues. AAV2 exhibits tropism for kidney tissue. AAV7, AAV8, and AAV9 exhibit tropism for liver tissue. AAV4, AAV5, AAV6, and AAV9 exhibits tropism for lung tissue. AAV8 exhibits tropism for pancreas cells. AAV3, AAV5, and AAV8 show tropism for photoreceptor cells. AAV1, AAV2, AAV4, AAV5, and AAV8 exhibit tropism for retinal pigment epithelium (RPE) cells. AAV1. AAV6, AAV7, AAV8, and AAV9 show tropism for skeletal muscle. It has been shown that mutating several tyrosine residues on the AAV2 capsid significantly enhanced neuronal transduction in the striatum and hippocampus, and the ablation of heparin sulfate (HS) binding also increased the volumetric spread of the vector (Kanaan et al., Mol Ther Nucleic Acids. 2017 Sep. 15; 8:184-19). AAV2 variants with heparin sulfate proteoglycan (HSPG) mutation (AAV2-HBKO, AAVT-TT, AAV44-9) have been shown to have enhanced neural and brain transduction.

Further modification to the virus can be performed to enhance the efficiency of gene transfer, for example, by improving the tropism of each serotype. One approach is to swap domains from one serotype capsid to another, and thus create hybrid vectors with desirable qualities from each parent. As the viral capsid is responsible for cellular receptor binding, the understanding of viral capsid domain(s) critical for binding is important. Mutation studies on the viral capsid (mainly on AAV2) performed before the availability of the crystal structure were mostly based on capsid surface functionalization by adsorption of exogenous moieties, insertion of peptide at a random position, or comprehensive mutagenesis at the amino acid level. Choi, et al. Curr Gene Ther. 2005 June; 5 (3): 299-310, describe different approaches and considerations for hybrid serotypes.

Capsids from other AAV serotypes offer advantages in certain in vivo applications over rAAV vectors based on the AAV2 capsid. First, the appropriate use of rAAV vectors with particular serotypes may increase the efficiency of gene delivery in vivo to certain target cells that are poorly infected, or not infected at all, by AAV2 based vectors. Secondly, it may be advantageous to use rAAV vectors based on other AAV serotypes if re-administration of rAAV vector becomes clinically necessary. It has been demonstrated that re-administration of the same rAAV vector with the same capsid can be ineffective, possibly due to the generation of neutralizing antibodies generated to the vector. Xiao, et al. 1999; Halbert, et al. 1997. This problem may be avoided by administration of a rAAV particle whose capsid is composed of proteins from a different AAV serotype, not affected by the presence of a neutralizing antibody to the first rAAV vector. Xiao, et al. 1999. For the above reasons, recombinant AAV vectors constructed using cap genes from serotypes including and in addition to AAV2 are desirable. It will be recognized that the construction of recombinant HSV vectors similar to rHSV but encoding the cap genes from other AAV serotypes, e.g., AAV1, AAV2, AAV3, AAV5 to AAV9, is achievable using the methods described herein to produce rHSV, In certain preferred embodiments of the invention as described herein, recombinant AAV vectors constructed using cap genes from different AAV are preferred. The significant advantages of construction of these additional rHSV vectors are ease and savings of time, compared with alternative methods used for the large-scale production of rAAV. In particular, the difficult process of constructing new rep and cap inducible cell lines for each different capsid serotypes is avoided.

IV. Making Recombinant AAV (rAAV) Vectors

The production, purification, and characterization of the rAAV vectors of the present invention may be carried out using any of the many methods known in the art. For reviews of laboratory-scale production methods, see, e.g., Clark R K, Recent advances in recombinant adeno-associated virus vector production. Kidney Int. 61s: 9-15 (2002); Choi V W et al., Production of recombinant adeno-associated viral vectors for In vitro and in vivo use. Current Protocols in Molecular Biology 16.25.1-16.25.24 (2007) (hereinafter Choi et al.); Grieger J C & Samulski R J, Adeno-associated virus as a gene therapy vector: Vector development, production, and clinical applications. Adv Biochem Engin/Biotechnol 99:119-145 (2005) (hereinafter Grieger & Samulski); Heilbronn R & Weger S, Viral Vectors for Gene Transfer: Current Status of Gene Therapeutics, in M. Schäfer-Korting (ed.), Drug Delivery, Handbook of Experimental Pharmacology, 197:143-170 (2010) (hereinafter Heilbronn); Howarth J L et al., Using viral vectors as gene transfer tools. Cell Biol Toxicol 26:1-10 (2010) (hereinafter Howarth). The production methods described below are intended as non-limiting examples.

AAV vector production may be accomplished by cotransfection of packaging plasmids. Heilbronn. The cell line supplies the deleted AAV genes rep and cap and the required helper virus functions. The adenovirus helper genes, VA-RNA, E2A and E4 are transfected together with the AAV rep and cap genes, either on two separate plasmids or on a single helper construct. A recombinant AAV vector plasmid wherein the AAV capsid genes are replaced with a transgene expression cassette (comprising the gene of interest, e.g., a PGRN nucleic acid; a promoter; and minimal regulatory elements) bracketed by ITRs, is also transfected. These packaging plasmids are typically transfected into 293 cells, a human cell line that constitutively expresses the remaining required Ad helper genes, E1A and E1B. This leads to amplification and packaging of the AAV vector carrying the gene of interest.

Multiple serotypes of AAV, including 12 human serotypes and more than 100 serotypes from nonhuman primates have now been identified. Howarth et al. Cell Biol Toxicol 26:1-10 (2010). The AAV vectors of the present invention may comprise capsid sequences derived from AAVs of any known serotype. As used herein, a “known serotype” encompasses capsid mutants that can be produced using methods known in the art. Such methods, include, for example, genetic manipulation of the viral capsid sequence, domain swapping of exposed surfaces of the capsid regions of different serotypes, and generation of AAV chimeras using techniques such as marker rescue. See Bowles et al. Marker rescue of adeno-associated virus (AAV) capsid mutants: A novel approach for chimeric AAV production. Journal of Virology, 77 (1): 423-432 (2003), as well as references cited therein. Moreover, the AAV vectors of the present invention may comprise ITRs derived from AAVs of any known serotype. Preferentially, the ITRs are derived from one of the human serotypes AAV1-AAV12. According to some embodiments of the present invention, a pseudotyping approach is employed, wherein the genome of one ITR serotype is packaged into a different serotype capsid.

According to some embodiments, the capsid sequences are derived from one of the human serotypes AAV1-AAV12. According to some embodiments, the capsid sequences are derived from AAV1. AAV2, AAV3, AAV4, AAV5, AAV6, AAV7, AAV8, AAV9, AAV10, AAV11, AAV12, AAVrh8, AAVrh10, AAV2.retro, and variants or hybrids thereof (e.g. AAV2 variants with HSPG mutation, (AAV2-HBKO, AAVT-TT, AAV44.9), AAV 1+9 hybrids). According to some embodiments, the particular capsid sequences confer enhanced neural and brain transduction. According to some embodiments, the capsid sequences are derived from an AAV2 variant with high tropism for targeting cells of the CNS (e.g., neuronal cells, astrocytes). According to some embodiments, the AAV is AAV9. According to some embodiments, the AAV is AAVrh10.

AAV tropism is determined by the specific interaction between distinct viral capsid proteins and their cognate cellular receptors. Thus, a rAAV having a capsid appropriate for the tissue being targeted can be selected. According to some embodiments, recombinant AAV vectors can be directly targeted by genetic manipulation of the viral capsid sequence, particularly in the looped out region of the AAV three-dimensional structure, or by domain swapping of exposed surfaces of the capsid regions of different serotypes, or by generation of AAV chimeras using techniques such as marker rescue. See Bowles et al. Marker rescue of adeno-associated virus (AAV) capsid mutants: A novel approach for chimeric AAV production. Journal of Virology, 77 (1): 423-432 (2003), as well as references cited therein.

One possible protocol for the production, purification, and characterization of recombinant AAV (rAAV) vectors is provided in Choi et al. Generally, the following steps are involved: design a transgene expression cassette, design a capsid sequence for targeting a specific receptor, generate adenovirus-free rAAV vectors, purify and titer. These steps are summarized below and described in detail in Choi et al.

The transgene expression cassette may be a single-stranded AAV (ssAAV) vector or a “dimeric” or self-complementary AAV (scAAV) vector that is packaged as a pseudo-double-stranded transgene. Choi et al.; Howarth et al. Using a traditional ssAAV vector generally results in a slow onset of gene expression (from days to weeks until a plateau of transgene expression is reached) due to the required conversion of single-stranded AAV DNA into double-stranded DNA. In contrast, scAAV vectors show an onset of gene expression within hours that plateaus within days after transduction of quiescent cells. Heilbronn. According to some embodiments, a scAAV is used, where the scAAV has rapid transduction onset and increased stability compared to single stranded AAV. Alternatively, the transgene expression cassette may be split between two AAV vectors, which allows delivery of a longer construct. See e.g., Daya S. and Berns, K. I., Gene therapy using adeno-associated virus vectors. Clinical Microbiology Reviews, 21 (4): 583-593 (2008) (hereinafter Daya et al.). A ssAAV vector can be constructed by digesting an appropriate plasmid (such as, for example, a plasmid containing the PGRN gene) with restriction endonucleases to remove the rep and cap fragments, and gel purifying the plasmid backbone containing the AAVwt-ITRs. Choi et al. Subsequently, the desired transgene expression cassette can be inserted between the appropriate restriction sites to construct the single-stranded rAAV vector plasmid. A scAAV vector can be constructed as described in Choi et al.

Then, a large-scale plasmid preparation (at least 1 mg) of the rAAV vector and the suitable AAV helper plasmid and pXX6 Ad helper plasmid can be purified by double CsCl gradient fractionation. Choi et al. A suitable AAV helper plasmid may be selected from the pXR series, pXR1-pXR5, which respectively permit cross-packaging of AAV2 ITR genomes into capsids of AAV serotypes 1 to 5. The appropriate capsid may be chosen based on the efficiency of the capsid's targeting of the cells of interest. Known methods of varying genome (i.e., transgene expression cassette) length and AAV capsids may be employed to improve expression and/or gene transfer to specific cell types (e.g., retinal cone cells). See, e.g., Yang G S, Virus-mediated transduction of murine retina with adeno-associated virus: Effects of viral capsid and genome size. Journal of Virology, 76 (15): 7651-7660.

Next, 293 cells are transfected with pXX6 helper plasmid, rAAV vector plasmid, and AAV helper plasmid. Choi et al. Subsequently the fractionated cell lysates are subjected to a multistep process of rAAV purification, followed by either CsCl gradient purification or heparin sepharose column purification. The production and quantitation of rAAV virions may be determined using a dot-blot assay. In vitro transduction of rAAV in cell culture can be used to verify the infectivity of the virus and functionality of the expression cassette.

In addition to the methods described in Choi et al., various other transfection methods for production of AAV may be used in the context of the present invention. For example, transient transfection methods are available, including methods that rely on a calcium phosphate precipitation protocol.

In addition to the laboratory-scale methods for producing rAAV vectors, the present invention may utilize techniques known in the art for bioreactor-scale manufacturing of AAV vectors, including, for example, Heilbronn; Clement, N. et al. Large-scale adeno-associated viral vector production using a herpesvirus-based system enables manufacturing for clinical studies. Human Gene Therapy, 20:796-606.

Advances toward achieving the desired goal of scalable production systems that can yield large quantities of clinical grade rAAV vectors have largely been made in production systems that utilize transfection as a means of delivering the genetic elements needed for rAAV production in a cell. For example, removal of contaminating adenovirus helper has been circumvented by replacing adenovirus infection with plasmid transfection in a three-plasmid transfection system in which a third plasmid comprises nucleic acid sequences encoding adenovirus helper proteins (Xiao, et al. 1998), Improvements in two-plasmid transfection systems have also simplified the production process and increased rAAV vector production efficiency (Grimm, et al. 1998).

Several strategies for improving yields of rAAV from cultured mammalian cells are based on the development of specialized producer cells created by genetic engineering. In one approach, production of rAAV on a large scale has been accomplished by using genetically engineered “proviral” cell lines in which an inserted AAV genome can be “rescued” by infecting the cell with helper adenovirus or HSV. Proviral cell lines can be rescued by simple adenovirus infection, offering increased efficiency relative to transfection protocols.

A second cell-based approach to improving yields of rAAV from cells involves the use of genetically engineered “packaging” cell lines that harbor in their genomes either the AAV rep and cap genes, or both the rep-cap and the ITR-gene of interest (Qiao, et al. 2002). In the former approach, in order to produce rAAV, a packaging cell line is either infected or transfected with helper functions, and with the AAV ITR-GOI elements. The latter approach entails infection or transfection of the cells with only the helper functions. Typically, rAAV production using a packaging cell line is initiated by infecting the cells with wild-type adenovirus, or recombinant adenovirus. Because the packaging cells comprise the rep and cap genes, it is not necessary to supply these elements exogenously.

rAAV yields from packaging cell lines have been shown to be higher than those obtained by proviral cell line rescue or transfection protocols.

Improved yields of rAAV have been made using approaches based on delivery of helper functions from herpes simplex virus (HSV) using recombinant HSV amplicon systems. Although modest levels of rAAV vector yield, of the order of 150-500 viral genomes (vg) per cell, were initially repotted (Conway, et al. 1997), more recent improvements in rHSV amplicon-based systems have provided substantially higher yields of rAAV v.g. and infectious particles (ip) per cell (Feudner, et al. 2002). Amplicon systems are inherently replication-deficient; however the use of a “gutted” vector, replication-competent (rcHSV), or replication-deficient rHSV still introduces immunogenic HSV components into rAAV production systems. Therefore, appropriate assays for these components and corresponding purification protocols for their removal must be implemented.

In addition to these methods, methods for producing recombinant AAV viral particles in a mammalian cell are described herein comprising co-infecting a mammalian cell capable of growing in suspension with a first recombinant herpesvirus comprising a nucleic acid sequence encoding an AAV rep and an AAV cap gene each operably linked to a promoter, and a second recombinant herpesvirus comprising a PGRN gene, and a promoter operably linked to said PGRN gene, flanked by AAV inverted terminal repeats to facilitate packaging of the gene of interest, and allowing the virus to infect the mammalian cell, thereby producing recombinant AAV viral particles in a mammalian cell.

Any type of mammalian cell that is capable of supporting replication of herpesvirus is suitable for use according to the methods of the invention as described herein. Accordingly, the mammalian cell can be considered a host cell for the replication of herpesvirus as described in the methods herein. Any cell type for use as a host cell is contemplated by the present invention, as long as the cell is capable of supporting replication of herpesvirus. Examples of suitable genetically unmodified mammalian cells include but are not limited to cell lines such as HEK-293 (293), Vero, RD, BHK-21, HT-1080, A549, Cos-7, ARPE-19, and MRC-5.

The host cells used in the various embodiments of the present invention may be derived, for example, from mammalian cells such as human embryonic kidney cells or primate cells. Other cell types might include, but are not limited to BHK cells, Vero cells, CHO cells or any eukaryotic cells for which tissue culture techniques are established as long as the cells are herpesvirus permissive. The term “herpesvirus permissive” means that the herpesvirus or herpesvirus vector is able to complete the entire intracellular virus life cycle within the cellular environment. In certain embodiments, methods as described occur in the mammalian cell line BHK, growing in suspension. The host cell may be derived from an existing cell line, e.g., from a BHK cell line, or developed de novo.

The methods for producing a rAAV gene construct described herein include also a recombinant AAV viral particle produced in a mammalian cell by the method comprising co-infecting a mammalian cell capable of growing in suspension with a first recombinant herpesvirus comprising a nucleic acid encoding an AAV rep and an AAV cap gene each operably linked to a promoter; and (ii) a second recombinant herpesvirus comprising a PGRN, and a promoter operably linked to said PGRN gene; and allowing the virus to infect the mammalian cell, and thereby producing recombinant AAV viral particles in a mammalian cell. As described herein, the herpesvirus is a virus selected from the group consisting of: cytomegalovirus (CMV), herpes simplex (HSV) and varicella zoster (VZV) and epstein barr virus (EBV). The recombinant herpesvirus is replication defective. According to some embodiments, the AAV cap gene has a serotype selected from the group consisting of AAV1, AAV2, AAV3, AAV4, AAV5, AAV6, AAV7. AAV8, AAV9, AAV10, AAV11, AAV12, AAVrh8, AAVrh10, AAV2.retro, and variants or hybrids thereof (e.g. AAV2 variants with HSPG mutation, (AAV2-HBKO, AAVT-TT, AAV44.9), AAV 1+9 hybrids). According to some embodiments, the particular capsid sequences confer enhanced neural and brain transduction. According to some embodiments, the AAV is AAV9. According to some embodiments, the AAV is AAVrh10.

U.S. Patent Application Publication No. 2007/0202587, incorporated by reference in its entirety herein, describes required elements of rAAV Production Systems. Recombinant AAV is produced In vitro by introduction of gene constructs into cells known as producer cells. Known systems for production of rAAV employ three fundamental elements: (1) a gene cassette containing the gene of interest, (2) a gene cassette containing AAV rep and cap genes and (3) a source of “helper” virus proteins.

The first gene cassette is constructed with the gene of interest flanked by inverted terminal repeats (ITRs) from AAV. ITRs function to direct integration of the gene of interest into the host cell genome and are essential for encapsidation of the recombinant genome. Hermonat and Muzyczka, 1984; Samulski et al. 1983. The second gene cassette contains rep and cap, AAV genes encoding proteins needed for replication and packaging of rAAV. The rep gene encodes four proteins (Rep 78, 68, 52 and 40) required for DNA replication. The cap genes encode three structural proteins (VP1, VP2, and VP3) that make up the virus capsid. Muzyczka and Berns, 2001.

The third element is required because AAV does not replicate on its own. Helper functions are protein products from helper DNA viruses that create a cellular environment conducive to efficient replication and packaging of rAAV. Traditionally, adenovirus (Ad) has been used to provide helper functions for rAAV, but herpesviruses can also provide these functions as discussed herein.

Production of rAAV vectors for gene therapy is carried out In vitro, using suitable producer cell lines such as BHK cells grown in suspension. Other cell lines suitable for use in the invention include HEK-293 (293), Vero, RD, BHK-21, HT-1080, A549, Cos-7, ARPE-19, and MRC-5.

Any cell type can be used as a host cell, as long as the cell is capable of supporting replication of a herpesvirus. One of skill in the art would be familiar with the wide range of host cells that can be used in the production of herpesvirus from host cells. Examples of suitable genetically unmodified mammalian host cells, for example, may include but are not limited to cell lines such as HEK-293 (293), Vero, RD, BHK-21, HT-1080, A549, Cos-7, ARPE-19, and MRC-5.

A host cell may be adapted for growth in suspension culture. The host cells may be Baby Hamster Kidney (BHK) cells. BHK cell line grown in suspension is derived from an adaptation of the adherent BHK cell line. Both cell lines are available commercially.

One strategy for delivering all of the required elements for rAAV production utilizes two plasmids and a helper virus. This method relies on transfection of the producer cells with plasmids containing gene cassettes encoding the necessary gene products, as well as infection of the cells with Ad to provide the helper functions. This system employs plasmids with two different gene cassettes. The first is a proviral plasmid encoding the recombinant DNA to be packaged as rAAV. The second is a plasmid encoding the rep and cap genes, To introduce these various elements into the cells, the cells are infected with Ad as well as transfected with the two plasmids. The gene products provided by Ad are encoded by the genes E1a, E1b, E2a, E4orf6, and Va. Samulski et al. 1998: Hauswirth et al. 2000; Muzyczka and Burns, 2001. Alternatively, in more recent protocols, the Ad infection step can be replaced by transfection with an adenovirus “helper plasmid” containing the VA, E2A and E4 genes. Xiao et al. 1998; Matsushita, et al. 1998.

While Ad has been used conventionally as the helper virus for rAAV production, other DNA viruses, such as herpes simplex virus type 1 (HSV-1) can be used as well. The minimal set of HSV-1 genes required for AAV2 replication and packaging has been identified, and includes the early genes UL5, UL8, UL52 and UL29. Muzyczka and Burns, 2001. These genes encode components of the HSV-1 core replication machinery, i.e., the helicase, primase, primase accessory proteins, and the single-stranded DNA binding protein. Knipe, 1989; Weller, 1991. This rAAV helper property of HSV-1 has been utilized in the design and construction of a recombinant herpes virus vector capable of providing helper virus gene products needed for rAAV production. Conway et al. 1999.

Production of rAAV vectors for gene therapy is carried out In vitro, using suitable producer cell lines such as BHK cells grown in suspension. Other cell lines suitable for use in the invention include HEK-293 (293), Vero, RD, BHK-21, HT-1080, A549, Cos-7, ARPE-19, and MRC-5.

Any cell type can be used as a host cell, as long as the cell is capable of supporting replication of a herpesvirus. One of skill in the art would be familiar with the wide range of host-cells that can be used in the production of herpesvirus from host cells. Examples of suitable genetically unmodified mammalian host cells, for example, may include but are not limited to cell lines such as HEK-293 (293), Vero, RD, BHK-21, HT-1080, A549, Cos-7, ARPE-19, and MRC-5.

A host cell may be adapted for growth in suspension culture. In certain embodiments of the present invention, the host cells are Baby Hamster Kidney (BHK) cells. BHK cell line grown in suspension is derived from an adaptation of the adherent BHK cell line. Both cell lines are available commercially.

rHSV-Based rAAV Manufacturing Process

Methods for the production of recombinant AAV viral particles in cells growing in suspension are described herein. Suspension or non-anchorage dependent cultures from continuous established cell lines are the most widely used means of large scale production of cells and cell products. Large scale suspension culture based on fermentation technology has clear advantages for the manufacturing of mammalian cell products. Homogeneous conditions can be provided in the bioreactor which allows for precise monitoring and control of temperature, dissolved oxygen, and pH, and ensure that representative samples of the culture can be taken. The rHSV vectors used are readily propagated to high titer on permissive cell lines both in tissue culture flasks and bioreactors, and provided a production protocol amenable to scale-up for virus production levels necessary for clinical and market production.

Cell culture in stirred tank bioreactors provides very high volume-specific culture surface area and has been used for the production of viral vaccines (Griffiths, 1986). Furthermore, stirred tank bioreactors have industrially been proven to be scalable. One example is the multiplate CELL CUBE cell culture system. The ability to produce infectious viral vectors is increasingly important to the pharmaceutical industry, especially in the context of gene therapy.

Growing cells according to methods described herein may be done in a bioreactor that allows for large scale production of fully biologically-active cells capable of being infected by the Herpes vectors of the present invention. Bioreactors have been widely used for the production of biological products from both suspension and anchorage dependent animal cell cultures. Most large-scale suspension cultures are operated as batch or fed-batch processes because they are the most straightforward to operate and scale up. However, continuous processes based on chemostat or perfusion principles are available. The bioreactor system may be set up to include a system to allow for media exchange. For example, filters may be incorporated into the bioreactor system to allow for separation of cells from spent media to facilitate media exchange. According to some embodiments of the present methods for producing Herpes virus, media exchange and perfusion is conducted beginning on a certain day of cell growth. For example, media exchange and perfusion can begin on day 3 of cell growth. The filter may be external to the bioreactor, or internal to the bioreactor.

A method for producing recombinant AAV viral particles may comprise: co-infecting a suspension cell with a first recombinant herpesvirus comprising a nucleic acid encoding an AAV rep and an AAV cap gene each operably linked to a promoter; and a second recombinant herpesvirus comprising a PGRN gene construct, and a promoter operably linked to said gene of interest; and allowing the cell to produce the recombinant AAV viral particles, thereby producing the recombinant AAV viral particles. The cell may be HEK-293 (293), Vero, RD, BHK-21, HT-1080, A549, Cos-7, ARPE-19, and MRC-5. According to some embodiments, the cap gene may be selected from an AAV with a serotype selected from the group consisting of AAV1, AAV2, AAV3, AAV4, AAV5, AAV6, AAV7, AAV8, AAV9, AAV10, AAV11, AAV12, AAVrh8, AAVrh10, AAV2.retro, and variants or hybrids thereof (e.g. AAV2 variants with HSPG mutation, (AAV2-HBKO, AAVT-TT. AAV44.9), AAV 1+9 hybrids). According to some embodiments, the particular capsid sequences confer enhanced neural and brain transduction. According to some embodiments, the AAV is AAV9. According to some embodiments, the AAV is AAVrh10. The cell may be infected at a combined multiplicity of infection (MOI) of between 3 and 14. The first herpesvirus and the second herpesvirus may be viruses selected from the group consisting of: cytomegalovirus (CMV), herpes simplex (HSV) and varicella zoster (VZV) and Epstein Barr Virus (EBV). The herpesvirus may be replication defective. The co-infection may be simultaneous.

According to some embodiments, the recombinant AAV viral particles further comprise a Woodchuck Hepatitis Virus Posttranscriptional Regulatory Element (WPRE).

A method for producing recombinant AAV viral particles in a mammalian cell may comprise co-infecting a suspension cell with a first recombinant herpesvirus comprising a nucleic acid encoding an AAV rep and an AAV cap gene each operably linked to a promoter; and a second recombinant herpesvirus comprising a PGRN gene construct, and a promoter operably linked to said PGRN gene construct; and allowing the cell to propagate, thereby producing the recombinant AAV viral particles, whereby the number of viral particles produced is equal to or greater than the number of viral particles grown in an equal number of cells under adherent conditions. The cell may be HEK-293 (293), Vero, RD, BHK-21, HT-1080, A549, Cos-7, ARPE-19, and MRC-5. The cap gene may be selected from an AAV with a serotype selected from the group consisting of AAV1, AAV2, AAV3, AAV4, AAV5, AAV6, AAV7, AAV8, AAV9, AAV10, AAV11, AAV12, AAVrh8, AAVrh10, AAV2.retro, and variants or hybrids thereof (e.g. AAV2 variants with HSPG mutation, (AAV2-HBKO, AAVT-TT. AAV44.9), AAV 1+9 hybrids). According to some embodiments, the AAV is AAVrh10. According to some embodiments, the AAV is AAV9. According to some embodiments, the particular capsid sequences confer enhanced neural and brain transduction. The cell may be infected at a combined multiplicity of infection (MOI) of between 3 and 14. The first herpesvirus and the second herpesvirus may be viruses selected from the group consisting of: cytomegalovirus (CMV), herpes simplex (HSV) and varicella zoster (VZV) and Epstein Barr Virus (EBV). The herpesvirus may be replication defective. The co-infection may be simultaneous.

A method for delivering a nucleic acid sequence encoding a therapeutic protein to a suspension cell, the method comprising: co-infecting the BHK cell with a first recombinant herpesvirus comprising a nucleic acid encoding an AAV rep and an AAV cap gene each operably linked to a promoter; and a second herpesvirus comprising a PGRN gene construct, wherein the gene of interest comprises a therapeutic protein coding sequence, and a promoter operably linked to said PGRN gene; and wherein said cell is infected at a combined multiplicity of infection (MOI) of between 3 and 14; and allowing the virus to infect the cell and express the therapeutic protein, thereby delivering the nucleic acid sequence encoding the therapeutic protein to the cell. The cell may be HEK-293 (293), Vero, RD, BHK-21, HT-1080, A549, Cos-7, ARPE-19, and MRC-5. See, e.g., U.S. Pat. No. 9,783,826.

### V. Methods of Treatment

### AAV and Gene Therapy

Gene therapy refers to treatment of inherited or acquired diseases by replacing, altering, or supplementing a gene responsible for the disease. It is achieved by introduction of a corrective gene or genes into a host cell, generally by means of a vehicle or vector. Gene therapy using rAAV holds great promise for the treatment of many diseases. A method of producing recombinant adeno-associated virus (rAAV), and in particular producing large quantities of recombinant AAV, to support treatment of neurodegenerative diseases are described herein.

To date more than 500 gene therapy clinical trials have been conducted worldwide. Efforts to use rAAV as a vehicle for gene therapy hold promise for its applicability as a treatment for human diseases. Already, some success has been achieved pre-clinically, using recombinant AAV (rAAV) for the delivery and long-term expression of introduced genes into cells in animals, including clinically important non-dividing cells of the brain, liver, skeletal muscle and lung. In some tissues, AAV vectors have been shown to integrate into the genome of the target cell. Hirata, et al. 2000, J. of Virology 74:4612-4620.

An additional advantage of rAAV is its ability to perform this function in non-dividing cell types including hepatocytes, neurons and skeletal myocytes. rAAV has been used successfully as a gene therapy vehicle to enable expression of erythropoietin in skeletal muscle of mice (Kessler, et al. 1996), tyrosine hydroxylase and aromatic amino acid decarboxylase in the CNS in monkey models of Parkinson disease (Kaplitt, et al. 1994) and Factor IX in skeletal muscle and liver in animal models of hemophilia. At the clinical level, the rAAV vector has been used in human clinical trials to deliver the CFTR gene to cystic fibrosis patients and the Factor IX gene to hemophilia patients (Flotte, et al. 1998; Wagner, et al. 1998), Further, AAV is a helper-dependent DNA parvovirus, which is not associated with disease in humans or mammals (Berns and Bohensky, 1987, Advances in Virus Research, Academic Press Inc, 32:243-307). Accordingly, one of the most important attributes of AAV vectors is their safety profile in phase I clinical trials.

AAV gene therapy has been carried out in a number of different pathological settings and to treat a various diseases and disorders. For example, in a phase I study, administration of an AAV2-FIX vector into the skeletal muscle of eight hemophilia B subjects proved safe and achieved local gene transfer and Factor IX expression for at least 10 months after vector injection (Jiang, et al. Mol Ther. 14 (3): 452-5 2006), a phase I trial of intramuscular injection of a recombinant adeno-associated virus alpha 1-antitrypsin (rAAV2-CB-hAAT) gene vector to AAT-deficient adults has been described previously (Flotte, et al. Hum Gene Ther. 2004 15 (1): 93-128), and in another clinical trial AA V-GAD gene therapy of the subthalamic nucleus has been shown to be safe and well tolerated by patients with advanced Parkinson's disease (Kaplitt et al. Lancet. 200723; 369 (9579): 2097-105).

### Progranulin and Neurodegenerative Diseases

Methods are provided herein that can be used to treat neurodegenerative diseases in a subject. According to some embodiments, the neurodegenerative disease is mediated by a heritable mutation in the subject. According to some embodiments, the neurodegenerative disease is mediated by an environmental insult to the subject. As used herein, a neurodegenerative disease mediated by an environmental insult to the patient means a disease that is caused by an environmental insult and is not caused by a heritable mutation of the progranulin gene that modifies progranulin expression. A heritable mutation is a permanent mutation in a patient's DNA that may be transmitted to the patient's offspring. Delivery of one or more of the nucleic acids described herein to CNS cells, and in particular to neuronal cells, can be used to treat neurodegenerative diseases.

According to some embodiments, methods are provided herein that employ PGRN AAV-based gene therapy for treating a progranulin-associated neurodegenerative disease including but not limited to familial frontotemporal dementia (FTD), frontotemporal lobar degeneration (FTLD), neuronal ceroid lipofuscinosis (NCL), including neuronal ceroid lipofuscinosis-11 (CLN11) and Batten disease, and Alzheimer's disease (AD). According to some embodiments, methods are provided herein that employ PGRN AAV-based gene therapy for preventing progranulin-associated neurodegenerative disorder including but not limited to familial frontotemporal dementia (FTD), frontotemporal lobar degeneration (FTLD), neuronal ceroid lipofuscinosis (NCL), including neuronal ceroid lipofuscinosis-11 (CLN11) and Batten disease, and Alzheimer's disease (AD).

The methods described herein allow for the production of recombinant AAV viral particles in a mammalian cell comprising co-infecting a mammalian cell capable of growing in suspension with a first recombinant herpesvirus and a second recombinant herpesvirus comprising a progranulin gene construct that has therapeutic value in the treatment of progranulin-associated neurodegenerative disorder including but not limited to familial frontotemporal dementia (FTD) and neuronal ceroid lipofuscinosis-11 (CLN11).

The gene therapy constructs described herein may be used in methods and compositions for the treatment and/or prevention of progranulin-associated neurodegenerative disorders. Progranulin-associated neurodegenerative disorders include, but are not limited to, 20% of the incidences of familial frontotemporal dementia (FTD) and all instances of neuronal ceroid lipofuscinosis-11 (CLN11). neurodegenerative disorder including but not limited to familial frontotemporal dementia (FTD), frontotemporal lobar degeneration (FTLD), neuronal ceroid lipofuscinosis (NCL), including neuronal ceroid lipofuscinosis-11 (CLN11) and Batten disease, and Alzheimer's disease (AD).

Progranulin, a secreted glycoprotein, is encoded in humans by the single GRN gene. Progranulin (PGRN) is predominantly expressed by microglia in the brain. Progranulin (PGRN) is a secreted 593 amino acid multifunction protein that is highly conserved and found in a wide range of species ranging from eukaryotes to humans. PGRN is widely distributed throughout the CNS where it is found primarily in neurons and microglia but has also been detected, at much lower levels, in astrocytes and oligodendrocytes. Progranulin consists of seven and a half, tandemly repeated, non-identical copies of the 12 cysteine granulin motif. Many cellular processes and diseases are associated with this unique pleiotropic factor that include, but are not limited to, embryogenesis, tumorigenesis, inflammation, wound repair, neurodegeneration and lysosome function. Haploinsufficiency caused by autosomal dominant mutations within the GRN gene leads to frontotemporal lobar degeneration, a progressive neuronal atrophy that presents in patients as frontotemporal dementia. Frontotemporal dementia is an early onset form of dementia, distinct from Alzheimer's disease. The GRN-related form of frontotemporal lobar dementia is a proteinopathy characterized by the appearance of neuronal inclusions containing ubiquitinated and fragmented TDP-43 (encoded by TARDBP). Chitramuthu et al. Brain 2017 140 (12): 3081-3104; Suárez-Calvet et al. EMBO Molecular Medicine 2018 e9712.

The progranulin (PGRN) AAV construct described herein provides a gene therapy vehicle for the treatment of PGRN-associated neurodegenerative disorders, including 20% of all incidences of familial frontotemporal dementia (FTD) and all instances of neuronal ceroid lipofucinosis-11 (CLN11). The PGRN AAV gene therapy construct and methods of use described herein provides a therapy for PGRN-associated neurodegenerative disorders, a long-felt unmet need as there are no gene therapy-based treatments available for patients suffering from PGRN-associated neurodegenerative disorders.

According to some embodiments, the PGRN AAV gene therapy is administered before the subject has developed a neurodegenerative disease. According to some embodiments, the subject is diagnosed with a neurodegenerative disease by molecular genetic testing to identify PGRN mutation. According to some embodiments, the subject has a family member with a neurodegenerative disease.=

The rAAV constructs described herein transduce CNS cells, and in particular neuronal cells, with greater efficiency than do conventional AAV vectors. According to some embodiments, the compositions and methods described herein enable the highly efficient delivery of nucleic acids to CNS cells, and in particular neuronal cells. According to some embodiments, the compositions and methods described herein enable the delivery to, and expression of, a transgene in at least 50%, (e.g., at least 50, 55, 60, 65, 70, 75, 80, 85, 90, 91, 92, 93, 94, 95, 96, 97, 98, or 99%) of inner hair cells or delivery to, and expression in, at least 50%, (e.g., at least 50, 55, 60, 65, 70, 75, 80, 85, 90, 91, 92, 93, 94, 95, 96, 97, 98, or 99) of neuronal cells. According to some embodiments, the compositions and methods described herein enable the delivery to, and expression of, a transgene in at least 70%, (e.g., at least 70, 75, 80, 85, 90, 91, 92, 93, 94, 95, 96, 97, 98, or 99%) of inner hair cells or delivery to, and expression in, at least 70%, (e.g., at least 70, 75, 80, 85, 90, 91, 92, 93, 94, 95, 96, 97, 98, or 99) of neuronal cells. According to some embodiments, the compositions and methods described herein enable the delivery to, and expression of, a transgene in at least 80%, (e.g., at least 80, 85, 90, 91, 92, 93, 94, 95, 96, 97, 98, or 99%) of inner hair cells or delivery to, and expression in, at least 80%, (e.g., at least 80, 85, 90, 91, 92, 93, 94, 95, 96, 97, 98, or 99) of neuronal cells.

According to some embodiments, the nucleic acid sequences described herein are directly introduced into a cell, where the nucleic acid sequences are expressed to produce the encoded product, prior to administration in vivo of the resulting recombinant cell. This can be accomplished by any of numerous methods known in the art, e.g., by such methods as electroporation, lipofection, calcium phosphate mediated transfection.

### VI. Pharmaceutical Compositions

According to some aspects, the disclosure provides pharmaceutical compositions comprising any of the vectors described herein, optionally in a pharmaceutically acceptable excipient.

As is well known in the art, pharmaceutically acceptable excipients are relatively inert substances that facilitate administration of a pharmacologically effective substance and can be supplied as liquid solutions or suspensions, as emulsions, or as solid forms suitable for dissolution or suspension in liquid prior to use. For example, an excipient can give form or consistency, or act as a diluent. Suitable excipients include but are not limited to stabilizing agents, wetting and emulsifying agents, salts for varying osmolarity, encapsulating agents, pH buffering substances, and buffers. Such excipients include any pharmaceutical agent suitable for direct delivery to the ear (e.g., inner ear or middle ear) which may be administered without undue toxicity. Pharmaceutically acceptable excipients include, but are not limited to, sorbitol, any of the various TWEEN compounds, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in REMINGTON'S PHARMACEUTICAL SCIENCES (Mack Pub. Co., N.J. 1991).

According to some embodiments, rAAV compositions are formulated to reduce aggregation of AAV particles in the composition, particularly where high rAAV concentrations are present. Methods for reducing aggregation of rAAVs are well known in the art and, include, for example, addition of surfactants, pH adjustment, salt concentration adjustment, etc. (See, e.g., Wright F R, et al., Molecular Therapy (2005) 12, 171-178, the contents of which are incorporated herein by reference.)

According to some embodiments, the pharmaceutical composition comprises one or more of BSST, PBS or BSS.

According to some embodiments, the pharmaceutical composition further comprises histidine buffer.

Although not required, the compositions may optionally be supplied in unit dosage form suitable for administration of a precise amount.

### VII. Methods of Administration

Generally, the compositions described herein are formulated for administration to the CNS. According to some embodiments, the compositions are formulated for administration to neuronal cells.

According to some embodiments, administration is intracerebral (e.g. to the intra-cisterna magna (ICM)), intrathecal (IT), with or without catheter, intravenous (IV), or a combination of IV and IT.

As used herein the term “intrathecal administration” refers to the administration of an agent, e.g., a composition comprising a rAAV, into the spinal canal. For example, intrathecal administration may comprise injection in the cervical region of the spinal canal, in the thoracic region of the spinal canal, or in the lumbar region of the spinal canal. Typically, intrathecal administration is performed by injecting an agent, e.g., a composition comprising a rAAV, into the subarachnoid cavity (subarachnoid space) of the spinal canal, which is the region between the arachnoid membrane and pia mater of the spinal canal. The subarachnoid space is occupied by spongy tissue consisting of trabecula (delicate connective tissue filaments that extend from the arachnoid mater and blend into the pia mater) and intercommunicating channels in which the cerebrospinal fluid is contained. According to some embodiments, intrathecal administration is not administration into the spinal vasculature.

As used herein, the term “intracerebral administration” refers to administration of an agent into and/or around the brain. Intracerebral administration includes, but is not limited to, administration of an agent into the cerebrum, medulla, pons, cerebellum, intracranial cavity, and meninges surrounding the brain. Intracerebral administration may include administration into the dura mater, arachnoid mater, and pia mater of the brain. Intracerebral administration may include, in some embodiments, administration of an agent into the cerebellomedullary (CM) cistern. Intracerebral administration may include, in some embodiments, administration of an agent into the cerebrospinal fluid (CSF) of the subarachnoid space surrounding the brain. Intracerebral administration may include, in some embodiments, administration of an agent into ventricles of the brain, e.g., the right lateral ventricle, the left lateral ventricle, the third ventricle, the fourth ventricle. According to some embodiments, intracerebral administration is not administration into the brain vasculature.

Intracerebral administration may involve direct injection into and/or around the brain. According to some embodiments, intracerebral administration involves injection using stereotaxic procedures. Stereotaxic procedures are well known in the art and typically involve the use of a computer and a 3-dimensional scanning device that are used together to guide injection to a particular intracerebral region, e.g., a ventricular region. Micro-injection pumps (e.g., from World Precision Instruments) may also be used. According to some embodiments, a microinjection pump is used to deliver a composition comprising a rAAV.

According to some embodiments, the infusion rate of the composition is 1 mL/minute or slower. According to some embodiments, the infusion rate of the composition is between about 10 μl/minute to 1000 μl/minute. As will be appreciated by the skilled artisan, infusion rates will depend on a variety of factors, including, for example, species of the subject, age of the subject, weight/size of the subject, serotype of the AAV, dosage required, intracerebral region targeted, etc. Thus, other infusion rates may be deemed by a skilled artisan to be appropriate in certain circumstances.

According to some embodiments, the composition comprising an rAAV as described herein is administered using an osmotic pump or an infusion pump. Both osmotic and infusion pumps are commercially available from a variety of suppliers, for example Alzet Corporation, Hamilton Corporation, Alza, Inc., Palo Alto, Calif.).

By safely and effectively transducing CNS cells as described herein, the methods of the invention may be used to treat an individual e.g., a human, wherein the transduced cells produce PGRN in an amount sufficient to treat or prevent a neurodegenerative disease.

According to the methods of treatment of the present invention, the volume of vector delivered may be determined based on the characteristics of the subject receiving the treatment, such as the age of the subject and the volume of the area to which the vector is to be delivered. According to some embodiments, the volume of the composition injected is between about 10 μl to about 1000 μl, or between about between about 100 μl to about 1000 μl, or between about between about 100 μl to about 500 μl, or between about 500 μl to about 1000 μl. According to some embodiments, the volume of the composition injected is more than about any one of 1 μl. 2 μl. 3 μl, 4 μl, 5 μl, 6 μl, 7 μl, 8 μl, 9 μl, 10 μl, 15 μl. 20 μl. 25 μl, 50 μl. 75 μl, 100 μl, 200 μl, 300 μl, 400 μl. 500 μl, 600 μl, 700 μl, 800 μl, 900 μl, or 1 mL, or any amount there between.

According to the methods of treatment of the present disclosure, the concentration of vector that is administered may differ depending on production method and may be chosen or optimized based on concentrations determined to be therapeutically effective for the particular route of administration. According to some embodiments, the concentration in vector genomes per milliliter (vg/ml) is selected from the group consisting of about 10⁸ vg/ml, about 10⁹ vg/ml, about 10¹⁰ vg/ml, about 10¹¹ vg/ml, about 10¹² vg/ml, about 10¹³ vg/ml, and about 10¹⁴ vg/ml. In preferred embodiments, the concentration is in the range of 10¹¹ vg/ml-10¹⁴ vg/ml in a volume of about 0.1 mL, about 0.2 mL, about 0.4 mL, about 0.6 mL, about 0.8 mL, and about 1.0 mL.

The effectiveness of the compositions described herein can be monitored by several criteria.

According to some embodiments, effectiveness of the compositions is determined by monitoring an improvement in subjects treated with PGRN rAAV. According to some embodiments, the effectiveness of the compositions described herein can be monitored in an in vivo mouse model. For example, in a PGRN−/−KO mouse model, increased PGRN protein levels in blood, CSF and brain tissue; decreased levels of neurofilament-1 (Nfl-1) in blood and CSF; decreased levels of lipofuscin and intracellular TDP43 from brain tissue may be indicators of the effectiveness of the compositions. According to some embodiments, the effectiveness of the compositions described herein can be monitored in human disease subjects. For example, increased PGRN protein levels in blood and CSF, decreased levels of Nfl-1 in blood and CSF, and behavioral and cognitive improvements may be used as indicators of the effectiveness of the compositions.

Further embodiments of the present invention will now be described with reference to the following examples. The examples contained herein are offered by way of illustration and not by any way of limitation.

### Examples

### Example 1. Methods

The invention was performed using, but not limited to, the following methods. The methods as described herein are set forth in PCT Application No. PCT/US2007/017645, filed on Aug. 8, 2007, entitled Recombinant AAV Production in Mammalian Cells, which claims the benefit of U.S. application Ser. No. 11/503,775, entitled Recombinant AAV Production in Mammalian Cells, filed Aug. 14, 2007, which is a continuation-in-part of U.S. application Ser. No. 10/252,182, entitled High Titer Recombinant AAV Production, filed Sep. 23, 2002, now U.S. Pat. No. 7,091,029, issued Aug. 15, 2006. The contents of all the aforementioned applications are hereby incorporated by reference in their entirety.

rHSV Co-Infection Method

The rHSV co-infection method for recombinant adeno-associated virus (rAAV) production employs two ICP27-deficient recombinant herpes simplex virus type 1 (rHSV-1) vectors, one bearing the AAV rep and cap genes (rHSV-rep2capX, with “capX” referring to any of the AAV serotypes), and the second bearing the gene of interest (GOI) cassette flanked by AAV inverted terminal repeats (ITRs). Although the system was developed with AAV serotype 2 rep, cap, and ITRs, as well as the humanized green fluorescent protein gene (GFP) as the transgene, the system can be employed with different transgenes and serotype/pseudo type elements.

Mammalian cells are infected with the rHSV vectors, providing all cis and trans-acting rAAV components as well as the requisite helper functions for productive rAAV infection. Cells are infected with a mixture of rHSV-rep2capX and rHSV-GOI. Cells are harvested and lysed to liberate rAAV-GOI, and the resulting vector stock is titered by the various methods described below.

### DOC-Lysis

At harvest, cells and media are separated by centrifugation. The media is set aside while the cell pellet is extracted with lysis buffer (20 mM Tris-HCl, pH 8.0, 150 mM NaCl) containing 0.5% (w/v) deoxycholate (DOC) using 2 to 3 freeze-thaw cycles, which extracts cell-associated rAAV. In some instances, the media and cell-associated rAAV lysate is recombined.

### In Situ Lysis

An alternative method for harvesting rAAV is by in situ lysis. At the time of harvest, MgCl₂ is added to a final concentration of 1 mM, 10% (v/v) Triton X-100 added to a final concentration of 1% (v/v), and Benzonase is added to a final concentration of 50 units/mL. This mixture is either shaken or stirred at 37° C. for 2 hours.

### Quantitative Real-Time PCR to Determine DRP Yield

The DNAse-resistant particle (DRP) assay employs sequence-specific oligonucleotide primers and a dual-labeled hybridizing probe for detection and quantification of the amplified DNA sequence using real-time quantitative polymerase chain reaction (qPCR) technology. The target sequence is amplified in the presence of a fluorogenic probe which hybridizes to the DNA and emits a copy-dependent fluorescence. The DRP titer (DRP/mL) is calculated by direct comparison of relative fluorescence units (RFUs) of the test article to the fluorescent signal generated from known plasmid dilutions bearing the same DNA sequence. The data generated from this assay reflect the quantity of packaged viral DNA sequences, and are not indicative of sequence integrity or particle infectivity.

### Green-Cell Infectivity Assay to Determine Infectious Particle Yield (rAA V-GFP Only)

Infectious particle (ip) titering is performed on stocks of rAA V-GFP using a green cell assay. C12 cells (a HeLa derived line that expressed AAV2 Rep and Cap genes-see references below) are infected with serial dilutions of rAA V-GFP plus saturating concentrations of adenovirus (to provide helper functions for AAV replication). After two to three days incubation, the number of fluorescing green cells (each cell representing one infectious event) are counted and used to calculate the ip/mL titer of the virus sample.

Clark K R et al. described recombinant adenoviral production in Hum. Gene Ther. 1995. 6:1329-1341 and Gene Ther. 1996. 3:1124-1132, both of which are incorporated by reference in their entireties herein.

TCID₅₀ to determine rAA V infectivity

Infectivity of rAAV particles harboring a gene of interest (rAAV-GOI) was determined using a tissue culture infectious dose at 50% (TCID₅₀) assay. Eight replicates of rAAV were serially diluted in the presence of human adenovirus type 5 and used to infect HeLaRC32 cells (a HeLa-derived cell line that expresses AAV2 rep and cap, purchased from ATCC) in a 96-well plate. At three days post-infection, lysis buffer (final concentrations of 1 mM Tris-HCl pH 8.0, 1 mM EDTA, 0.25% (w/v) deoxycholate, 0.45% (v/v) Tween-20, 0.1% (w/v) sodium dodecyl sulfate, 0.3 mg/mL Proteinase K) was added to each well then incubated at 37° C. for 1 h, 55° C. for 2 h, and 95° C. for 30 min. The lysate from each well (2.5 μL aliquot) was assayed in the DRP qPCR assay described above. Wells with Ct values lower than the value of the lowest quantity of plasmid of the standard curve were scored as positive. TCID₅₀ infectivity per mL (TCID₅₀/mL) was calculated based on the Karber equation using the ratios of positive wells at 10-fold serial dilutions.

### Cell Lines and Viruses

Production of rAAV vectors for gene therapy is carried out in vitro, using suitable producer cell lines such as HEK293 cells (293). Other cell lines suitable for use in the invention include Vero, RD, BHK-21, HT-1080, A549, Cos-7, ARPE-19, and MRC-5.

Mammalian cell lines were maintained in Dulbecco's modified Eagle's medium (DMEM, Hyclone) containing 2-10% (v/v) fetal bovine serum (FBS, Hyclone) unless otherwise noted. Cell culture and virus propagation were performed at 37° C., 5% CO2 for the indicated intervals.

### Infection Cell Density

Cells can be grown to various concentrations including, but not limited to at least about, at most about, or about 1×10⁶ to 4×10⁶ cells/mL. The cells can then be infected with recombinant herpesvirus at a predetermined MOI.

### Example 2. Cloning of the PGRN Expression Constructs

Successful AAV-Progranulin therapy for neurodegenerative disorders is critically dependent on the vector component. An optimal vector will provide an efficient capsid that targets the brain effectively, a moderate but cell-specific promoter and a stabilized transgene expressing human progranulin protein.

All constructs are variant iterations of the following:
pTR *-CBA*/hSYN1*-h/m (wt/co1,2,3,4,5,6,7) (ss*) PGRN (nothing/HA/SBI)-wpre-(SV/hGH) pA
*(potentially) optimized and/or sequence modulated elements

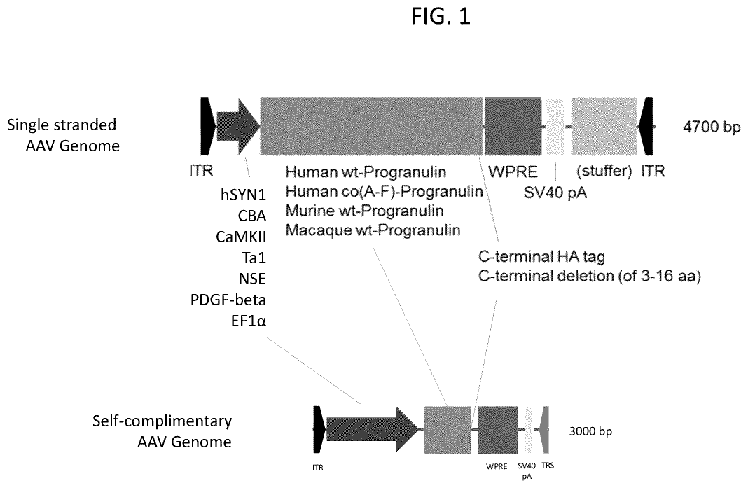

FIG. 1 shows a schematic of a PGRN construct, comprising inverted terminal repeats (ITR) at each end, a human synapsin 1 (hSYN1) or chicken beta actin (CBA) promoter, PGRN optionally comprising a C-terminal HA tag or a deletion of 3-16 nucleic acids in the C-terminal, Woodchuck Hepatitis Virus (WHP) Posttranscriptional Regulatory Element (WPRE), SV40 early polyadenylation signals (SV40 pA) and optionally stuffer DNA. The bottom panel of FIG. 1A shows a self-complementary AAV-genome.

Design and cloning of pTR-CBA-PGRN-WPRE: a Woodchuck Hepatitis Virus Posttranscriptional Regulatory Element (WPRE) site was inserted into the plasmid backbone.

Design and cloning of pTR-CBA-hGFP-WPRE: a control hGFP plasmid with CBA promoter was constructed, and at the same time, a multiple cloning site between GFP and WPRE in the plasmid backbone was inserted.

Design and cloning of pTR-hSyn-hGFP-WPRE: the hSyn promoter was inserted into the plasmid backbone and a control hGFP plasmid with hSyn promoter was constructed.

Design and cloning of pTR-CBA-hPGRNwt-HA-WPRE: a PGRN wild-type expression plasmid with HA tag and with CBA promoter was constructed.

Design and cloning of pTR-CBA-hPGRNwt-WPRE: a PGRN wild-type expression plasmid without HA tag and with CBA promoter was constructed.

Design and cloning of pTR-CBA-hPGRNco1-HA-WPRE: to construct a codon-optimized PGRN (ATUM, hPGRN_opt1_CpG reduced) expression plasmid with HA tag and with CBA promoter.

Design and cloning of pTR-CBA-hPGRNco2-HA-WPRE: a codon-optimized PGRN (ATUM, hPGRN_opt2_CpG reduced) expression plasmid with HA tag and with a CBA promoter was constructed.

Design and cloning of pTR-hSyn-hPGRNwt-HA-WPRE: a PGRN wild-type expression plasmid with HA tag and with hSyn promoter was constructed.

Design and cloning of pTR-hSyn-hPGRNwt-WPRE: a PGRN wild-type expression plasmid without HA tag and with hSyn promoter was constructed.

Design and cloning of pTR-hSyn-hPGRNco1-HA-WPRE: a codon-optimized PGRN (ATUM, hPGRN_opt1_CpG reduced) expression plasmid with HA tag and with hSyn promoter was constructed.

Design and cloning of pTR-hSyn-hPGRNco2-HA-WPRE: a codon-optimized PGRN (ATUM, hPGRN_opt2_CpG reduced) expression plasmid with HA tag and with hSyn promoter was constructed.

Design and cloning of pTR-CBA-hPGRNcoE-HA-WPRE: a codon-optimized PGRN (ATUM, hPGRN_opt1_CpG reduced) expression plasmid with HA tag with CBA promoter was constructed.

Design and cloning of pTR-CBA-hPGRNcoF-HA-WPRE: a codon-optimized PGRN (ATUM, hPGRN_opt2_CpG reduced) expression plasmid with HA tag with CBA promoter was constructed.

Design and cloning of pTR-hSyn-hPGRNcoE-HA-WPRE: a codon-optimized PGRN (ATUM, hPGRN_opt1_CpG reduced) expression plasmid with HA tag with hSyn promoter was constructed.

Design and cloning of pTR-hSyn-hPGRNcoF-HA-WPRE: a codon-optimized PGRN (ATUM, hPGRN_opt2_CpG reduced) expression plasmid with HA tag with hSyn promoter was constructed.

Design and cloning of pTR-hSyn-hPGRNcoEd5-WPRE and pTR-hSyn-hPGRNcoFd5-WPRE: codon-optimized PGRN (coEd5 and coFd5) expression plasmids with hSyn promoter with modification of C-terminal 5-amino acid truncation were constructed.

Exemplary nucleic acid sequences of the construct components are shown below:

| SEQ ID NO | Description |
|---|---|
| 1 | chicken beta actin (CBA) promoter |
| 2 | human synapsin 1 (hSYN1) promoter |
| 3 | AAV2 inverted terminal repeat (ITR) 5′ to 3′ |
| 4 | AAV2 inverted terminal repeat (ITR) 3′ to 5′ |
| 5 | wild type human progranulin (hPGRNwt) |
| 6 | wild type mouse progranulin (mPGRNwt) |
| 7 | macaque (Macaca mulatta) progranulin |
| 8 | hPGRNcoA |
| 9 | hPGRNcoB |
| 10 | hPGRNcoC |
| 11 | hPGRNcoD |
| 12 | hPGRNcoE |
| 13 | hPGRNcoF |
| 14 | hPGRN sortilin-binding deficient variant |
| 15 | hemagglutinin (HA) tag |
| 16 | WPRE |
| 17 | SV40 pA |
| 18 | calcium/calmodulin-dependent protein kinase II |
| | (CAMKII) promoter |
| 19 | Rat tubulin alpha 1 (Ta1) promoter |
| 20 | Rat neuron-specific enolase (NSE) promoter |
| 21 | human platelet-derived growth factor-beta chain |
| | (PDGF) promoter PDGFB_1 |
| 22 | EF1alpha promoter |
| 23 | CAG/CMV Enhancer |
| 24 | human platelet-derived growth factor-beta chain |
| | (PDGF-B) promoter PDGFB_2 |
| 25 | human platelet-derived growth factor-beta chain |
| | (PDGF) promoter PDGFB_3 |

### Promoter Vector Design and Synthesis:

Studies on in vitro comparison of strong-ubiquitous promoter (CBA) versus neuron-specific promoter (human Synapsin) were done to compare their specificity and acquired transgene expression levels. CBA promoter was obtained in a pAAV plasmid (pTR-CBA-PGRNwt-WPRE-pA). The sequence of PGRNwt was replaced with a sequence containing hGFP and a multiple cloning site using NEBuilder® HiFi DNA Assembly kit (New England Biolabs) to generate the plasmid pTR-CBA-hGFP-WPRE-pA. The human synapsin promoter sequence was synthesized commercially (Genscript), followed by in-house PCR amplification and extraction, resulting in a promoter segment with compatible restriction site segments for insertion into a unique viral packaging vector containing an ampicillin selection cassette, AAV ITR segments, hGFP reporter gene, WPRE and SV40 poly A (pTR-hSyn-hGFP-WPRE-pA).

Constructs were transformed into high efficiency E. coli cells (SURE2) for amplification, and clones were selected for validation. Sanger sequencing (Genewiz) and restriction digests (with appropriate restriction sites (Smal and Xmal) to check for promoter insertion and ITR integrity) were performed to validate the promoter plasmids. Positive clones for each construct were selected for subsequent experiments. Unique promoter constructs were tested for efficacy in driving hGFP expression via in vitro transfection of HEK-293 (control) or SHSY-5Y cells. In vitro data indicates that CBA and human synapsin promoters work in both tested cells with the CBA always stronger than synapsin promoter in both cells.

### Capsid Selection

Multiple immunohistological analyses indicate AAV9 and AAVrh10 both possess broad neuronal tropism favorable for therapy. AAVrh10 was chosen over AAV9 based on studies comparing AAV9 and AAVrh10 expression and efficacy in the central nervous system. AAVrh10 was found to transduce significantly better than AAV9. Also, AAV9 and rh10 have similarly low neutralizing Ab seroprevalences in the human population (18% and 21% respectively, Thwaite R et al. 2014).

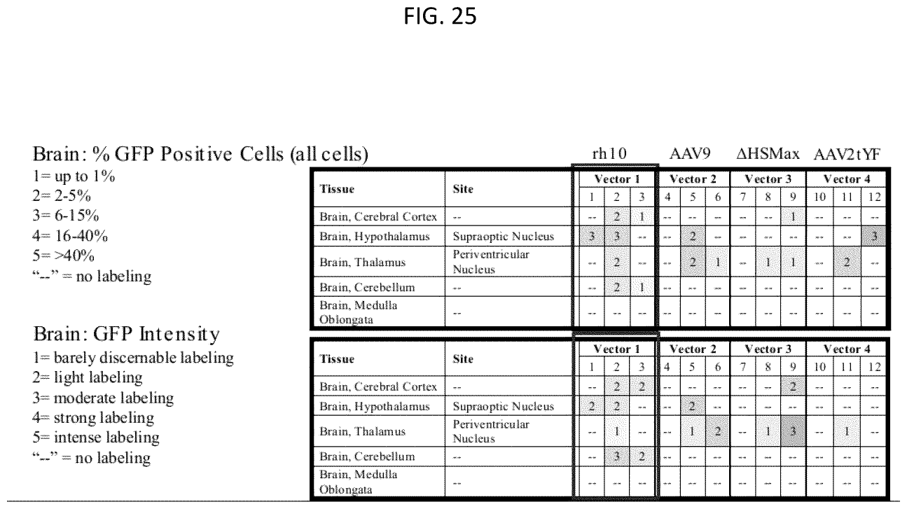

One set of experiments examined capsid selection in non-human primate (NHP) by intrathecal dosing. GFP constructs comprising AAVrh10, AAV9, ΔHSmax and AAV2tyf capsids were tested for GFP expression in the spinal cord and brain. FIG. 25 summarizes the results for each of the capsids tested, for percent GFP positive cells and GFP intensity in various regions of the brain after intrathecal injection. From this study, AAVrh10 showed the most GFP expression, followed by AAV9. All of the vectors were well-tolerated.

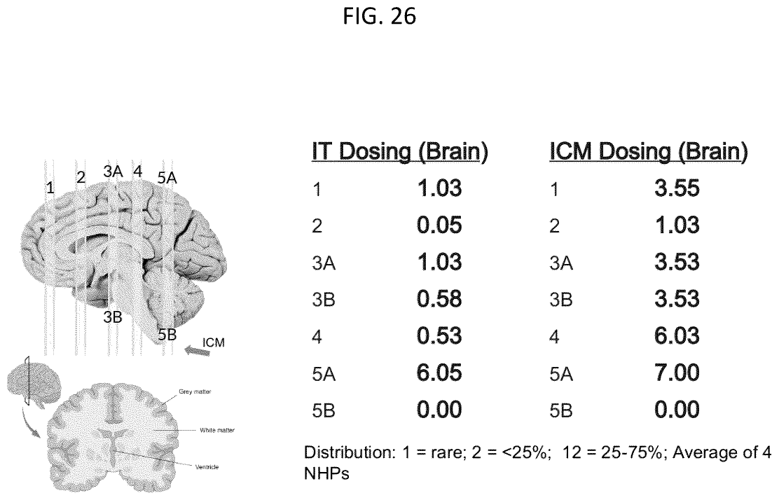

Next, AAVrh10 biodistribution in NHP by dosing into the cisterna magna (ICM) was carried out. It was found that 1.2E13 vg of AAVRh10 showed excellent biodistribution from frontal to dorsal NHP brain following ICM dosing. Scoring is shown in FIG. 26.

### Example 3. Promoter Selection

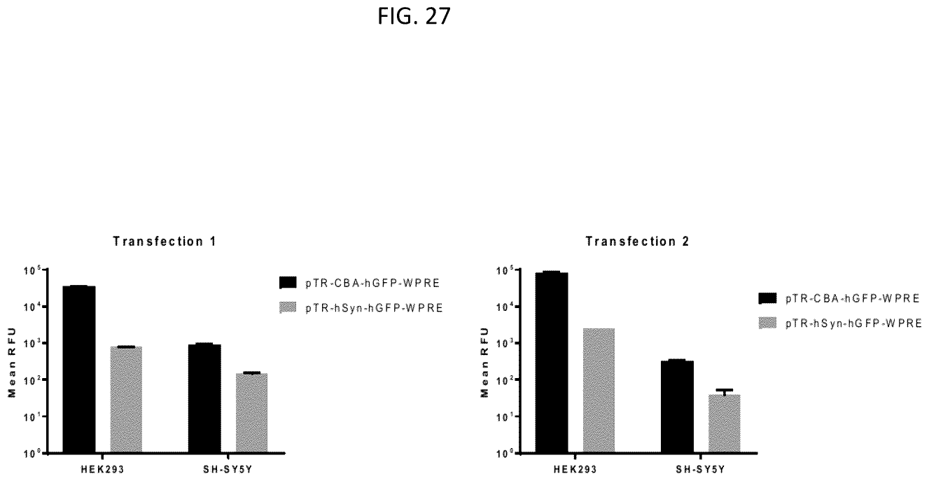

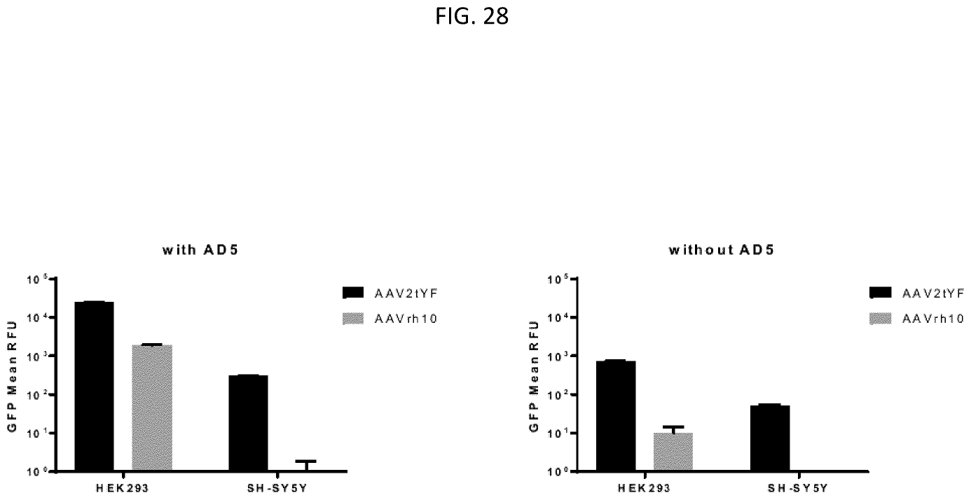

Experiments were carried out to test promoters for use in the progranulin vectors. The promoters employed were: a hSyn promoter (also called SYNP1, neuron specific) and a chimeric CMV-chicken β-actin promoter (CBA) promoter (ubiquitous). A first set of experiments were performed in vitro. As shown in FIG. 27, the CBA promoter drove stronger expression than SYNP1 in both Human embryonic kidney 293 cells (HEK293) and SH-SY5Y cells (confirmed using 2 different transfection reagents). SH-SY5Y is a cell line derived from the SK—N-SH neuroblastoma cell line, which frequently serves as a model for neurodegenerative disorders. FIG. 28 shows GFP expression after rAAVRh10-CBA-hGFP Transduction in HEK293 and SHSY-5Y cells, with and without AD5 vector. As shown in FIG. 28, in SH-SY5Y cells transduced with the AAVRh10-CBA construct, with and without AD5, no GFP was detected. The results shown in FIG. 27 demonstrate that both HEK and SH-SY5Y cells can be used to showcase WPRE-enhanced expression driven from either CBA or hSYN promoter. However, the level of hSYN-driven expression is considerably lower than CBA-driven expression in both cell types. The results shown in FIG. 28 demonstrate that either AAV serotype (AAV2tYF or AAVrh10) can be used to showcase transduction of vector in HEK cells (and is further enhanced by Ad5), but only AAV2tYF can be used to showcase transduction in SH-SY5Y cells (regardless of Ad5 addition).

These results indicated that for subsequent testing of AAVrh10 vectors in cell assays, HEK293 cells can be used, or if a neuron/neuron-like cell model will be used, a cell type other than SH-SY5Y may need to be used or conditions in SH-SY5Y cells may need to be further optimized.

Taken together, the results from the promoter selection work led to the selection of the SYNP1 promoter, because of its cell specificity, which is desirable to limit overexpression and hence possible toxicity. Moreover, it was shown that the SYNP1 promoter only increases PGRN in CSF, but not plasma, which is a further desirable characteristic of the constructs of the present invention.

### Example 4. Codon Optimized Progranulin Vector Design and Synthesis

Progranulin transgene optimization consists of efforts to enhance protein expression, stability, and function. Codon optimized variants of Progranulin (PGRN) were synthesized and assessed for changes in protein expression versus wildtype. Six codon optimized variants were generated, either with or without 27 bp C-terminal HA tag. The HA-tagged variants provide an alternative measure for protein expression, a means for discriminating over endogenous PGRN protein, and also allow the assessment of any therapeutic benefit to inhibiting PGRN-Sortilin binding.

Each codon optimized variant contains unique optimizations (i.e., codon usage, GC content, stability of 5′ mRNA structure, removal of RNA destabilizing sequences, etc.), that led to variants which vary in their DNA sequences but preserve their amino acid sequences. These variants were generated from one of three different optimization algorithms (or a combination thereof).

PCR amplification and extraction was performed, resulting in PGRN transgene segments with compatible restriction sites (NotI. NheI) for insertion into a unique viral packaging vector containing an ampicillin selection cassette and AAV ITR segments. Following full synthesis, codon optimized constructs were transformed into high efficiency E. coli cells (SURE2) for amplification, and clones were selected for validation. Sanger sequencing (Genewiz) and restriction digests (with appropriate restriction sites to check for transgene insertion and ITR integrity) were performed to validate the codon optimized PGRN and PGRNwt plasmids. Positive clones for each codon optimized construct were selected for subsequent experiments.

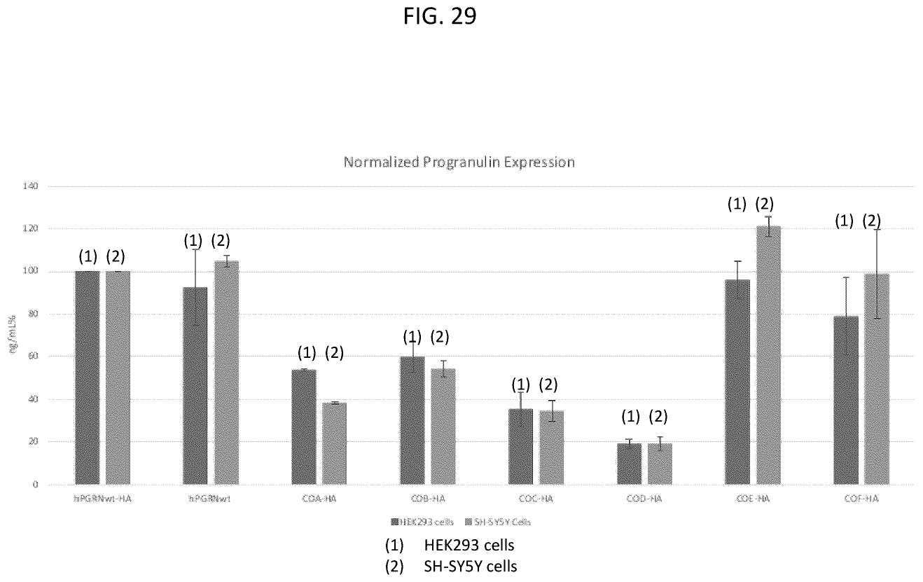

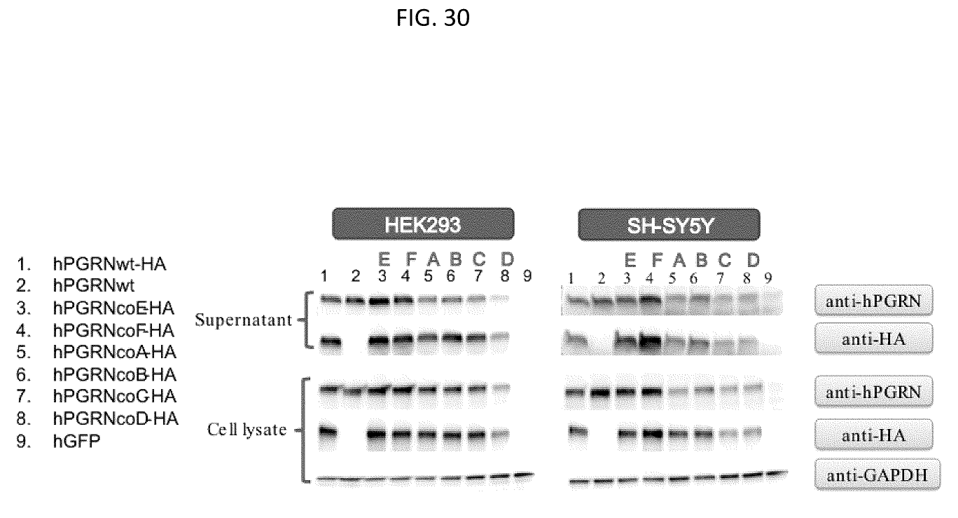

To determine how the 6 codon optimized variants (designated coA-F (e.g. PGRNcoA, PGRNcoB, PGRNcoC. PGRNcoD, PGRNcoE, PGRNcoF)) express PGRN compared to PGRNwt, transgene expression experiments were performed and analyzed via supernatant and cell lysate ELISA and western blotting. Both ELISA and Western results showed coE and coF variants had comparable protein expression to WT when assayed with Human Progranulin Quantikine ELISA Kit (R&D Systems) and when probed with anti-human progranulin antibody (Sigma) and anti-HA monoclonal antibody (ThermoFisher), while all other co variants displayed inferior expression (FIG. 29 and FIG. 30). Therefore, co-E and coF were selected as good candidates for transgene of choice. Both coE and coF migrate at the expected molecular weight of ˜80 kD on a SDS-PAGE (i.e. same as PGRNwt), as shown in FIG. 30.

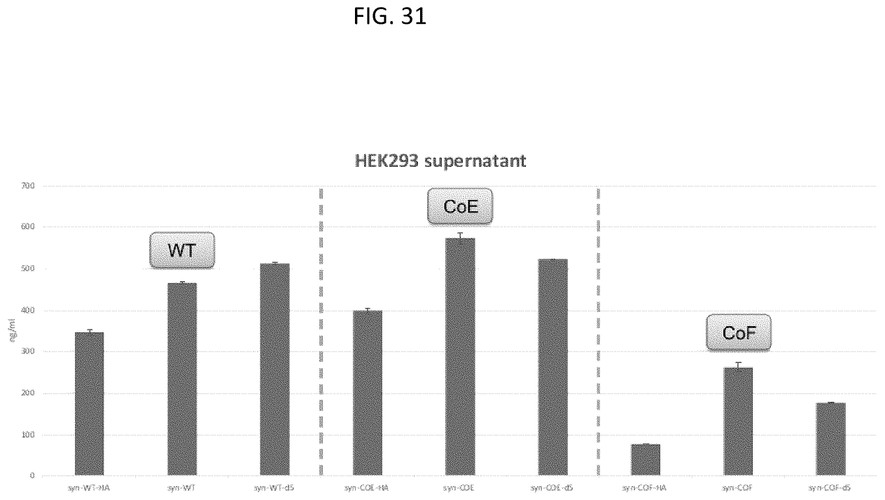

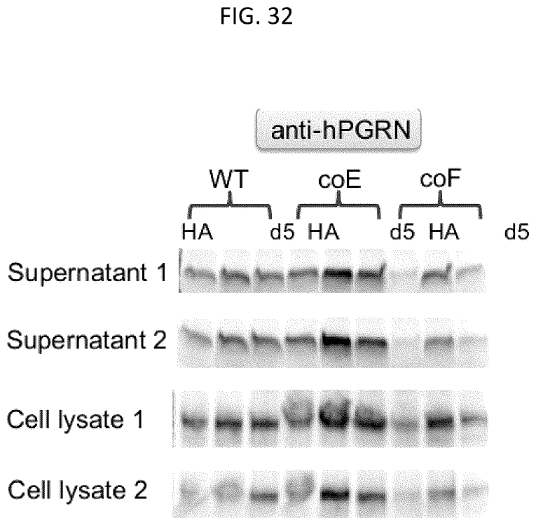

Next, codon optimized variants coE (PGRNcoE) and coF (PGRNcoF) under the control of hSYn promoter were transfected into HEK 293 cells and secreted transgene expression was analyzed via supernatant ELISA (FIG. 31). As shown in FIG. 31, coE showed comparable secreted progranulin expression (ng/ml) to WT, and higher secreted progranulin expression (ng/ml) than coF in HEK293 cells. Expression was also measured at day 5 (d5), where a slight decrease was seen with the variant coE and a greater decrease with the variant coF. Western blot results (FIG. 32) confirmed this result.

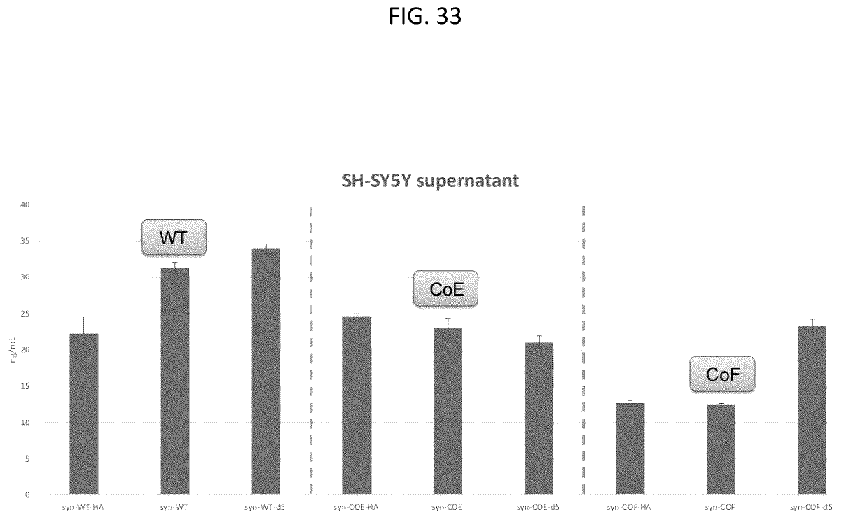

Similar experiments were performed in SH-SY5Y cells. The results are shown in FIG. 33. Codon optimized variants coE (PGRNcoE) and coF (PGRNcoF) under the control of hSYn promoter were transfected into SH-SY5Y cells and secreted transgene expression was analyzed via supernatant ELISA. As shown in FIG. 33, coE showed comparable secreted progranulin expression (ng/ml) to WT, and higher secreted progranulin expression (ng/ml) than coF in HEK293 cells. Expression was also measured at day 5 (d5), where a slight decrease in secreted progranulin expression was seen with the variant coE, while an increase was seen with the variant coF.

In addition to enhancing expression and stability toward optimization, avoidance of PGRN degradation to granulins and/or PGRN uptake by the cells has a great potential to increase PGRN availability. One of the approaches is to target PGRN binding to its main receptor, sortilin. Therefore, a vC-terminally modified PGRN of the selected candidates was generated by deleting 5 amino acid crucial for sortilin binding.

Experiments will be performed to see if PGRN expression can be enhanced through avoidance of binding to sortilin as the main receptor.

### Example 5. Production of rAAV-X Vectors

rAAV-X vectors were produced, where “X” is one of the following serotypes/variants: AAV-rh10, AAV-9 containing top selected genomic variants. Research grade preparation of these vectors will proceed by various methods. (1) In the first method, vectors are packaged by plasmid transfection of HEK293 cells and virus purified by iodixanol density gradient according to established methods (reference to this method can be found in Zolotukhin et al., 2002 Methods). (2) In the second method, vector is produced using AGTC's proprietary recombinant HSV complementation in suspension-cultured baby hamster kidney (sBHK) cells (Kang et al., Gene Ther 2009; Thomas et al., Hum Gene Ther 2009). A triple transfection method was used to make vector for all preclinical work. Triple transfection will also be used to make vector for GLP tox studies. Vector production is being optimized using the HAVE (HSV-associated) method and the method is expected to be used for production in later stage work, such as for preparation of clinical trial material. Studies will be carried out to demonstrate comparability of vector made by both methods.

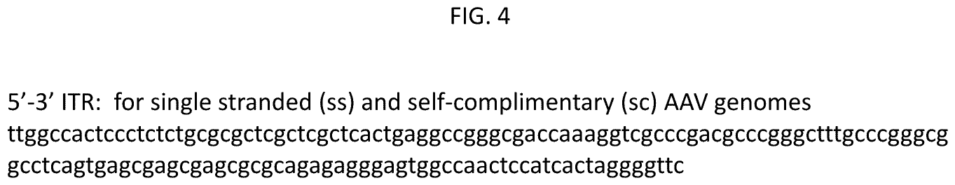

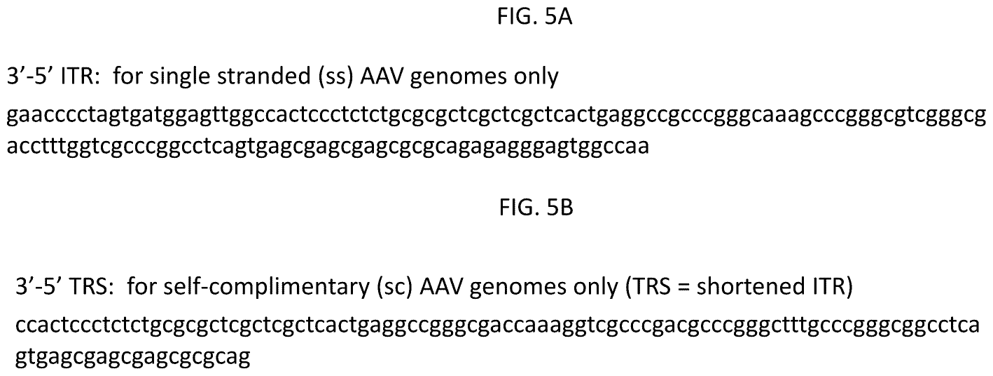

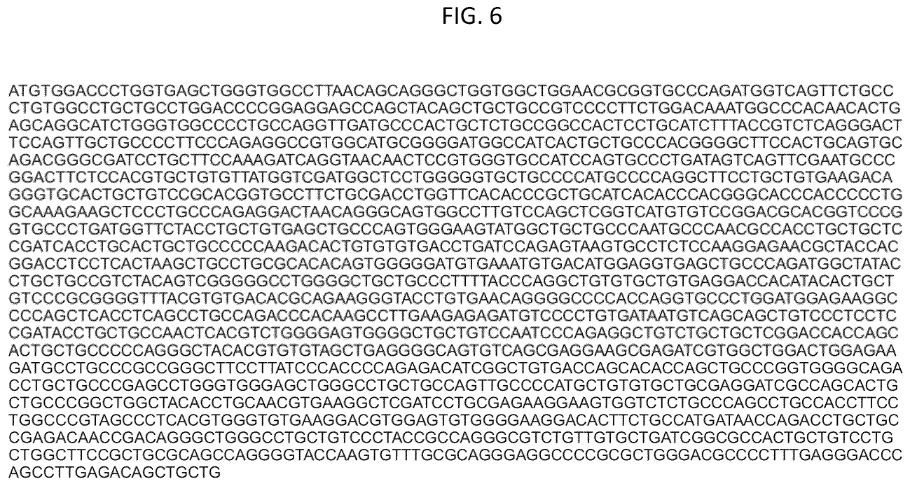

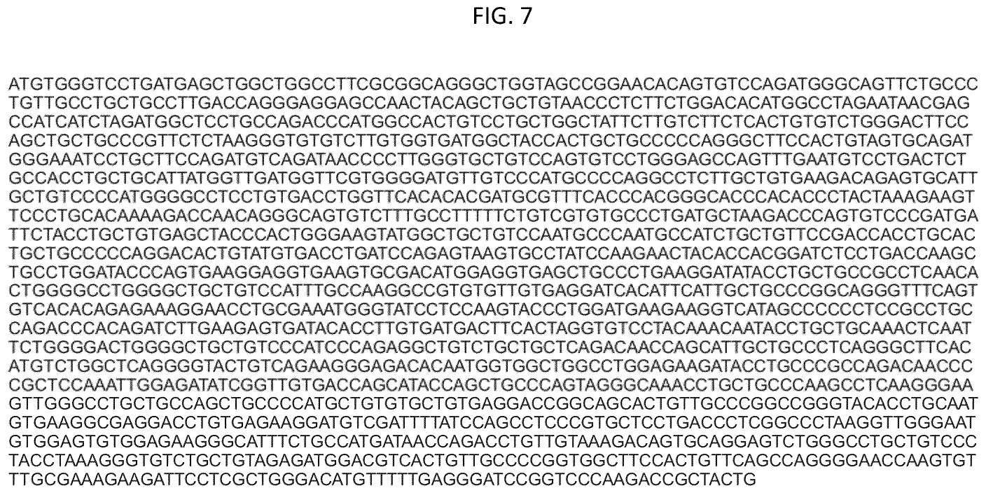

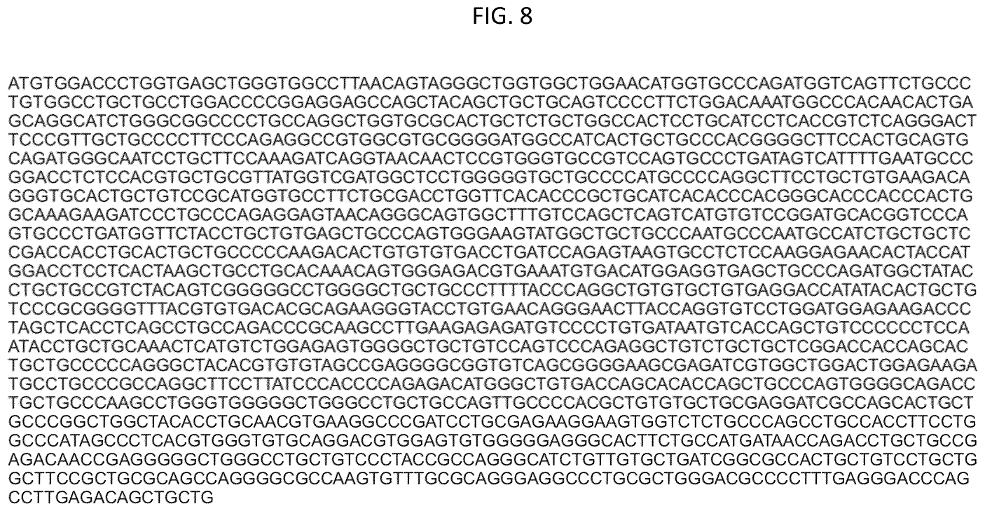

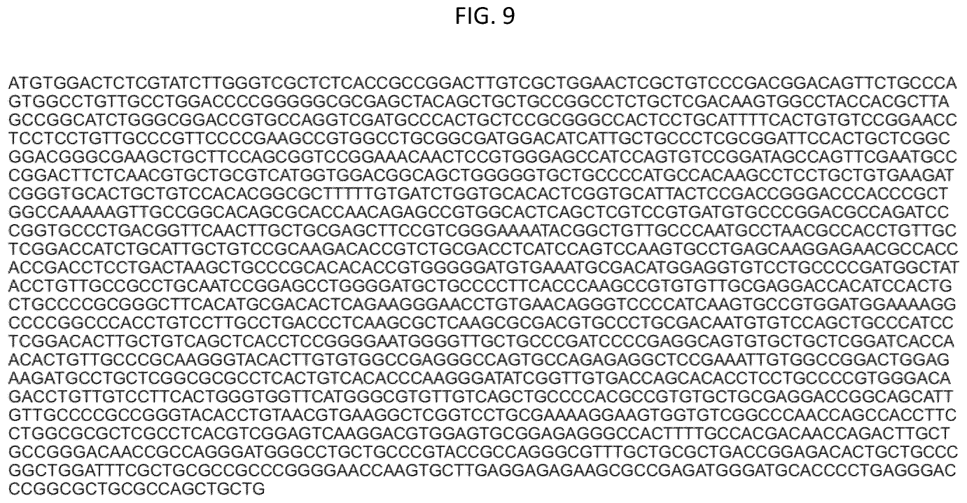

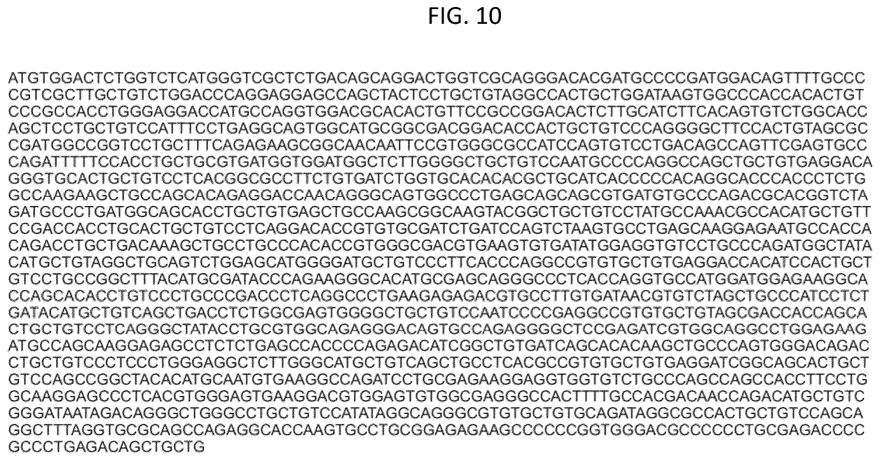

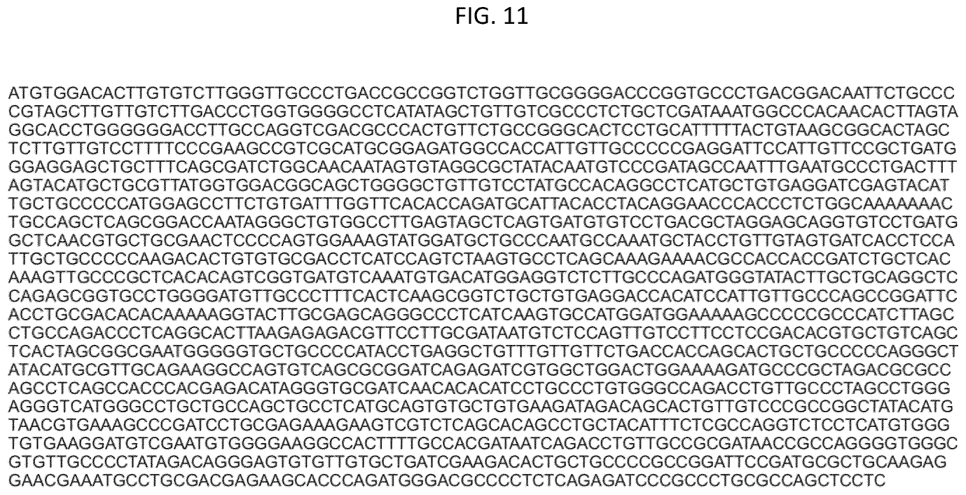

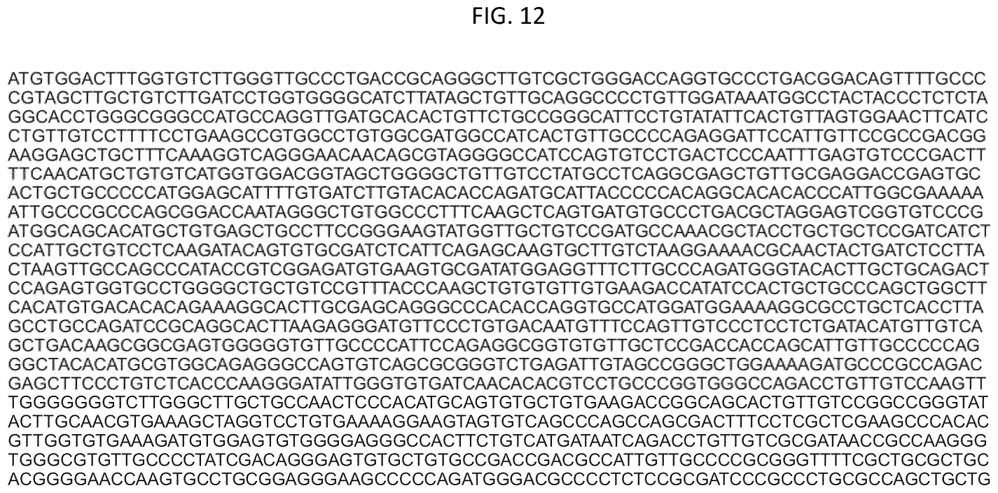

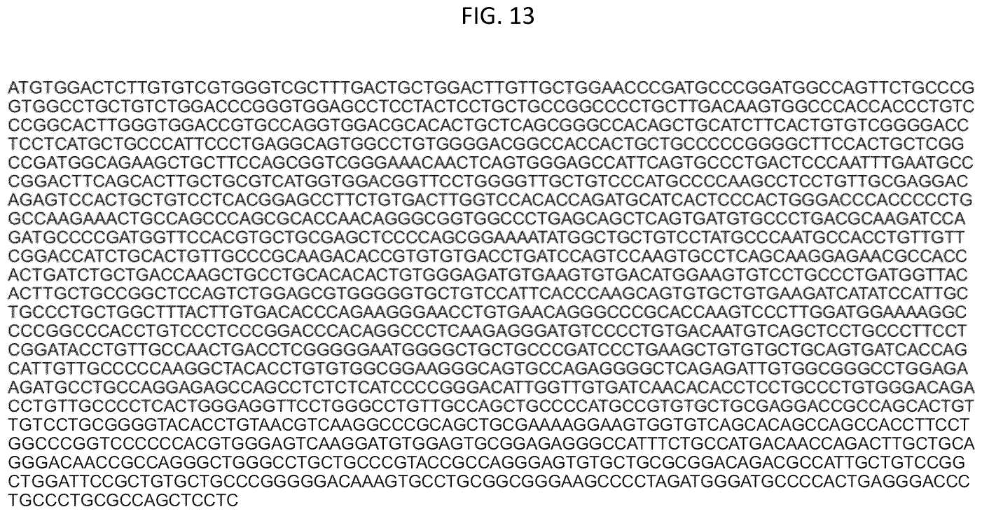

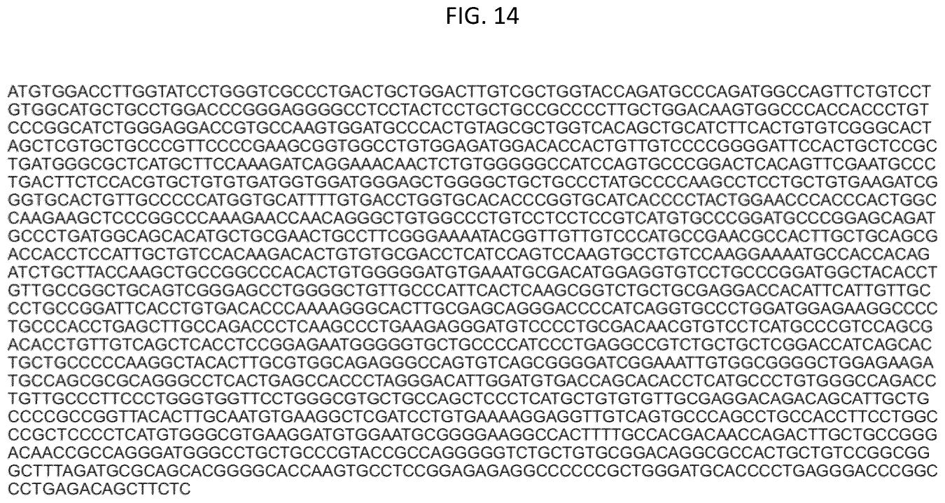

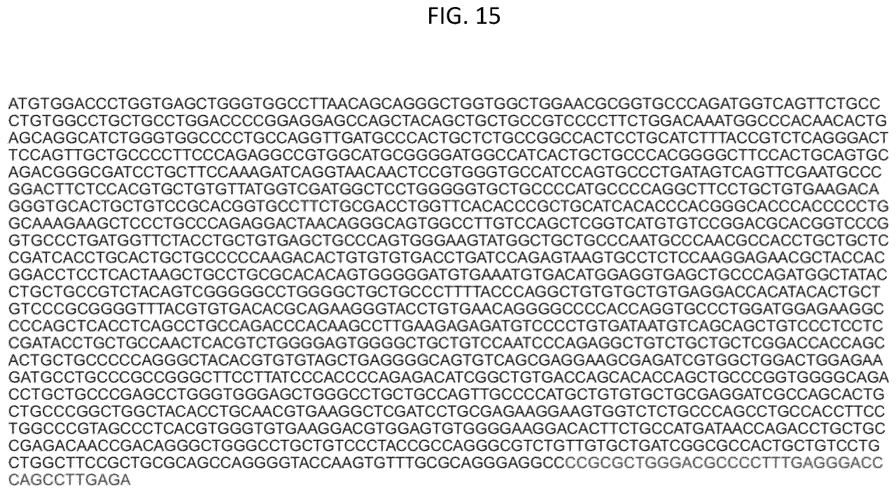

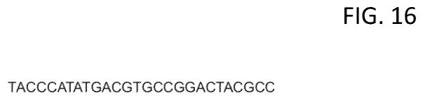

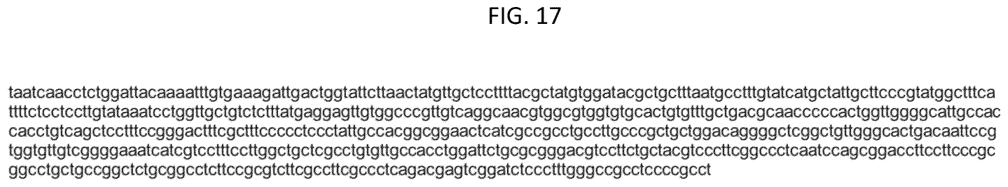

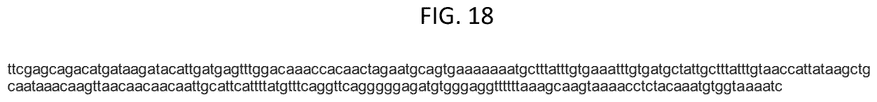

According to some embodiments, a vector construct comprising rAAVr10-SYN-PGRNwt was prepared by triple transfection, comprising the pUC-based “pTR” plasmid with the ITR—SYN-PGRNwt-wpre-pA-ITR cassette inserted. The nucleic acid sequence of the ITR—SYN-PGRNwt-wpre-pA-ITR cassette (FIG. 1) can be derived from individual sequences provided in FIG. 4 or FIGS. 5A and 5B (ITR sequences) or 5AB (for both ITRs), FIG. 3 (hSYN), FIG. 6 (hPGRNwt), FIG. 17 (for WPRE), and FIG. 18 (for SV40 pA).

### Example 6. In Vivo Studies: Progranulin Expression in Non-Human Primates

A study was conducted to evaluate systemic gene expression following a single intracisternal injection of a gene therapy (using rAAVr10-SYN-PGRNwt) in the cynomolgus monkey. To achieve this objective, two male cynomolgus monkeys, one of which was previously implanted with an intrathecal lumbar catheter for CSF sample collection (Animal 002A), were transferred to the study. The animals were administered a single dose of 1.5 mL of test article by cisterna magna puncture according to the following study design.

Study Design

| | Number | | | | Study |
|---|---|---|---|---|---|
| | of | | Dose | Concentration | Termination |
| Group | Animals | Test Article | (vg) | (vg/mL) | Day 57 |
| 1 | 2 | rAAVr10-SYN- | 5.84 × 10¹² | 3.89 × 10¹² | 2 |
| | Males | PGRNwt | | | Males |

Dose Administration: Both animals were sedated for dose administration via intrathecal cisterna magna (CM) puncture. Each animal was provided dexmedetomidine hydrochloride IM (0.04 mg/kg). At least 10 minutes later, an IM injection of ketamine hydrochloride (2.5 mg/kg) was provided to induce sedation. Once sedated, the animals were intubated and the cisterna magna area prepared for the spinal tap. The animals were placed in a lateral recumbent position as per NBR SOP. A micro incision was made in the skin using a 20-gauge needle before the introduction of the spinal needle to the cisterna magna. The spinal tap was performed utilizing a Gertie Marx® 22-gauge needle. Access to the CM was confirmed by the flow of CSF from the needle. Once CSF was observed in the hub of the spinal needle, the 1.5 mL dose was administered over approximately 1-2 minutes by manual bolus. Upon removing the needle, direct pressure was applied to the injection site followed by application of a topical aseptic ointment. The animals were placed in the Trendelenburg position for 10 minutes following dose administration, prior to anesthetic reversal. The reversal agent, atipamezole hydrochloride, was provided at a dose of 0.2 mg/kg IM, the time recorded and the animal returned to its cage.

In-life observations and measurements included clinical observations, body weight, and clinical pathology evaluations as described elsewhere in this report. Blood and CSF were collected for analysis. After collection of samples on Day 57, both animals were returned to the NBR primate stock colony.

There were no abnormal clinical signs or test article-related changes in body weight over the course of the study.

There were no meaningful changes in hematology or serum chemistry parameters at the time interval evaluated (Day 57) compared to prestudy.

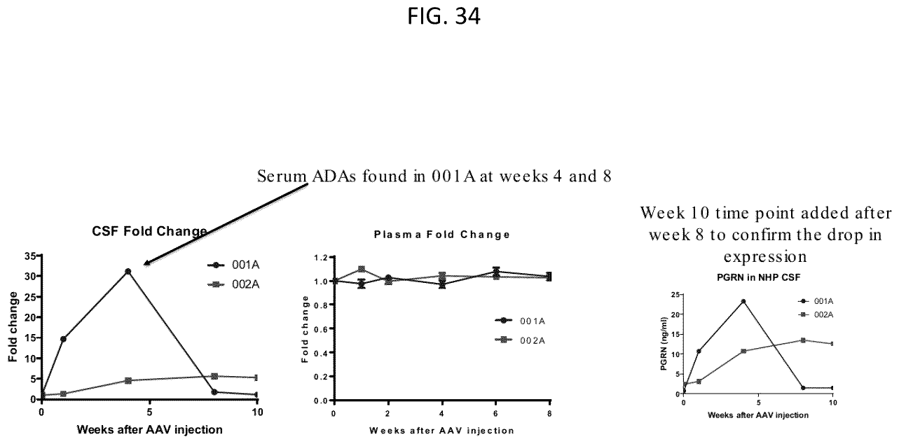

Following dosing with test article, the titers of anti-AAV neutralizing antibodies increased in all test samples. Results are shown in FIG. 34. Fold change in progranulin expression was determined in the cerebrospinal fluid (CSF) and the plasma over the course of 8 weeks or 10 weeks. As shown in FIG. 34, progranulin expression increased in the CSF, but not the plasma over the time course. Animal 001A developed a quicker/higher response, especially in CSF compared to Animal 002A. The week 10 time point was added to confirm the drop in CSF progranulin levels.

Although the invention has been described in some detail by way of illustration and example for purposes of clarity of understanding, it should be understood that certain changes and modifications may be practiced within the scope of the appended claims. Modifications of the above-described modes for carrying out the invention that would be understood in view of the foregoing disclosure or made apparent with routine practice or implementation of the invention to persons of skill in gene therapy, molecular biology, and/or related fields are intended to be within the scope of the following claims.

All publications (e.g., Non-Patent Literature), patents, patent application publications, and patent applications mentioned in this specification are indicative of the level of skill of those skilled in the art to which this invention pertains. All such publications (e.g., Non-Patent Literature), patents, patent application publications, and patent applications are herein incorporated by reference to the same extent as if each individual publication, patent, patent application publication, or patent application was specifically and individually indicated to be incorporated by reference.

While the foregoing invention has been described in connection with this preferred embodiment, it is not to be limited thereby but is to be limited solely by the scope of the claims which follow.

1. An isolated polynucleotide comprising a nucleic acid sequence encoding a mammalian progranulin, wherein the nucleic acid sequence further comprises an operably linked neuron-specific human synapsin-1 promoter (hSYN1).

2.-4. (canceled)

5. The polynucleotide of claim 1, wherein the nucleic acid sequence is at least 85% identical to a sequence selected from the group consisting of: SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13 and SEQ ID NO: 14.

6. The polynucleotide of claim 1, wherein the nucleic acid sequence comprises a sequence selected from the group consisting of: SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13 and SEQ ID NO: 14.

7. The polynucleotide of claim 1, wherein the nucleic acid sequence is codon optimized for mammalian expression.

8. (canceled)

9. (canceled)

10. The polynucleotide of claim 1, wherein the nucleic acid sequence further comprises:
an operably linked functionally optimized N-terminal signal sequence;
a hemagglutinin C-terminal tag;
a sortilin binding inhibitory (SBI) domain;
a 3′UTR regulatory region comprising a Woodchuck Hepatitis Virus Posttranscriptional Regulatory Element (WPRE); or
a polyadenylation signal.

11.-23. (canceled)

24. A transgene expression cassette comprising:
(a) the polynucleotide of claim 1; and
(b) minimal regulatory elements.

25. A nucleic acid vector comprising the expression cassette of claim 24.

26. (canceled)

27. A host cell comprising the transgene expression cassette of claim 24.

28. An expression vector comprising the polynucleotide of claim 1.

29. The expression vector of claim 28, wherein the vector is an adeno-associated viral (AAV) vector.

30. The vector of claim 29, wherein the serotype of the AAV vector is rhAAV10.

31. (canceled)

32. (canceled)

33. A recombinant adeno-associated (rAAV) expression vector comprising the polynucleotide of claim 1 wherein the polynucleotide is positioned between flanking inverted terminal repeats (ITRs).

34. (canceled)

35. The expression vector of claim 33, wherein the promoter is optimized to drive high progranulin expression.

36.-44. (canceled)

45. A composition comprising the polynucleotide of claim 1.

46.-48. (canceled)

49. A pharmaceutical composition comprising the expression vector of claim 28.

50. (canceled)

51. A method of treating or preventing a neurodegenerative disorder comprising administering the pharmaceutical composition of claim 49 to a subject in need thereof.

52.-53. (canceled)

54. A method of treating or preventing a neurodegenerative disorder comprising administering the recombinant adeno-associated (rAAV) expression vector of claim 33 to a subject in need thereof.

55. (canceled)

56. The method of claim 51, wherein:
the neurodegenerative disorder is characterized by cognitive disruption, behavioral impairment, deficient lysosomal storage, or a combination thereof;
the neurodegenerative disorder is a progranulin-associated neurodegenerative disorder;
the neurodegenerative disorder is selected from the group consisting of: familial frontotemporal dementia (FTD), frontotemporal lobar degeneration (FTLD), neuronal ceroid lipofuscinosis (NCL), and Alzheimer's disease (AD).

57.-59. (canceled)

60. The method of claim 51, wherein the administration is to the central nervous system.

61. The method of claim 51, wherein the route of administration is selected from the group consisting of: intravenous, intra cerebroventricular, intrathecal, and a combination thereof.

62.-66. (canceled)
